SEQUENCE LISTING (42 sequences)

SEQ ID NO: 1 - protein, 48 aa, Homo sapiens
ARGNDGARGSDGQPGPPGPPGTAGFPGSPGAKGEVGPAGSPGSNGAPG

SEQ ID NO: 2 - protein, 48 aa, Homo sapiens; identical to SEQ ID NO: 1

SEQ ID NO: 3 - protein, 45 aa, Homo sapiens
ERGEQGPAGSPGFQGLPGPAGPPGEAGKPGEQGVPGDLGAPGPSG

SEQ ID NO: 4 - protein, 39 aa, Homo sapiens
NTGAPGSPGVSGPKGDAGQPGEKGSPGAQGPPGAPGPLG

SEQ ID NO: 5 - protein, 35 aa, Homo sapiens
GPPGESGREGAPGAEGSPGRDGSPGAKGDRGETGP

SEQ ID NO: 6 - protein, 36 aa, Homo sapiens
RTGEVGAVGPPGFAGEKGPSGEAGTAGPPGTPGPQG

SEQ ID NO: 7 - protein, 32 aa, Homo sapiens
ESGREGAPGAEGSPGRDGSPGAKGDRGETGPA

SEQ ID NO: 8 - protein, 33 aa, Homo sapiens
LTGSPGSPGPDGKTGPPGPAGQDGRPGPPGPPG

SEQ ID NO: 9 - protein, 31 aa, Homo sapiens
ESGREGAPGAEGSPGRDGSPGAKGDRGETGP

SEQ ID NO: 10 - protein, 30 aa, Homo sapiens
ERGEAGIPGVPGAKGEDGKDGSPGEPGANG

SEQ ID NO: 11 - protein, 30 aa, Homo sapiens
KNGETGPQGPPGPTGPGGDKGDTGPPGPQG

SEQ ID NO: 12 - protein, 28 aa, Homo sapiens
NRGERGSEGSPGHPGQPGPPGPPGAPGP

SEQ ID NO: 13 - protein, 28 aa, Homo sapiens
NRGERGSEGSPGHPGQPGPPGPPGAPGP

SEQ ID NO: 14 - protein, 28 aa, Homo sapiens
GAPGQNGEPGGKGERGAPGEKGEGGPPG

SEQ ID NO: 15 - protein, 26 aa, Homo sapiens
ADGQPGAKGEPGDAGAKGDAGPPGPA

SEQ ID NO: 16 - protein, 27 aa, Homo sapiens
ANGAPGNDGAKGDAGAPGAPGSQGAPG

SEQ ID NO: 17 - protein, 25 aa, Homo sapiens
GNSGEPGAPGSKGDTGAKGEPGPVG

SEQ ID NO: 18 - protein, 25 aa, Homo sapiens
ADGQPGAKGEPGDAGAKGDAGPPGP

SEQ ID NO: 19 - protein, 24 aa, Homo sapiens
AGPPGEAGKPGEQGVPGDLGAPGP

SEQ ID NO: 20 - protein, 24 aa, Homo sapiens
NSGEPGAPGSKGDTGAKGEPGPVG

SEQ ID NO: 21 - protein, 24 aa, Homo sapiens
AGPPGEAGKPGEQGVPGDLGAPGP

SEQ ID NO: 22 - protein, 23 aa, Homo sapiens
GPPGEAGKPGEQGVPGDLGAPGP

SEQ ID NO: 23 - protein, 23 aa, Homo sapiens
GPPGEAGKPGEQGVPGDLGAPGP

SEQ ID NO: 24 - protein, 24 aa, Homo sapiens
DAGAPGAPGGKGDAGAPGERGPPG

SEQ ID NO: 25 - protein, 21 aa, Homo sapiens
NGDDGEAGKPGRPGERGPPGP

SEQ ID NO: 26 - protein, 22 aa, Homo sapiens
PPGEAGKPGEQGVPGDLGAPGP

SEQ ID NO: 27 - protein, 22 aa, Homo sapiens
SEGSPGHPGQPGPPGPPGAPGP

SEQ ID NO: 28 - protein, 20 aa, Homo sapiens
GLPGTGGPPGENGKPGEPGP

SEQ ID NO: 29 - protein, 20 aa, Homo sapiens
TGSPGSPGPDGKTGPPGPAG

SEQ ID NO: 30 - protein, 18 aa, Homo sapiens
DGAPGKNGERGGPGGPGP

SEQ ID NO: 31 - protein, 18 aa, Homo sapiens
SPGSPGPDGKTGPPGPAG

SEQ ID NO: 32 - protein, 17 aa, Homo sapiens
GDPGPPGPPGPPGPPAI

SEQ ID NO: 33 - protein, 17 aa, Homo sapiens
GSPGSPGPDGKTGPPGP

SEQ ID NO: 34 - protein, 16 aa, Homo sapiens
SPGSPGPDGKTGPPGP

SEQ ID NO: 35 - protein, 16 aa, Homo sapiens
GLPGTGGPPGENGKPG

SEQ ID NO: 36 - protein, 16 aa, Homo sapiens
SPGSPGPDGKTGPPGP

SEQ ID NO: 37 - protein, 14 aa, Homo sapiens
APGEDGRPGPPGPQ

SEQ ID NO: 38 - protein, 13 aa, Homo sapiens
SPGERGETGPPGP

SEQ ID NO: 39 - protein, 14 aa, Homo sapiens
SPGPDGKTGPPGPA

SEQ ID NO: 40 - protein, 13 aa, Homo sapiens
APGDRGEPGPPGP

SEQ ID NO: 41 - protein, 13 aa, Homo sapiens
SPGPDGKTGPPGP

SEQ ID NO: 42 - protein, 11 aa, Homo sapiens
APGDKGESGPS

(19) United States
(12) Patent Application Publication
(10) Pub. No.: US 2015/0099312 A1
(43) Pub. Date: Apr. 9, 2015

(54) PROCESS FOR NORMALIZING THE CONCENTRATION OF ANALYTES IN A URINE SAMPLE

(71) Applicant: Mosaiques Diagnostics and Therapeutices AG, Hannover (DE)

(72) Inventor: Harald Mischak, Sehnde (DE)

(21) Appl. No.: 14/516,378

(22) Filed: Oct. 16, 2014

Related U.S. Application Data
(63) Continuation of application No. 12/530,029, filed on Jan. 4, 2010, now abandoned.

(30) Foreign Application Priority Data
Mar. 7, 2007 (EP) 07103684.2

(51) Int. Cl. G01N 33/68; G01N 21/64; G01N 21/17; G01N 30/72; G01N 30/96

(52) U.S. Cl. CPC G01N 33/68; G01N 30/72; G01N 30/96; G01N 21/17; G01N 21/6486
USPC 436/501; 73/61.52; 250/282

(57) ABSTRACT

Process for normalizing the concentration of at least one analyte in a urine sample, comprising the following steps:
a) determining the concentration of at least one analyte;
b) determining the concentration of at least one collagen or collagen fragment;
c) normalizing the concentration of said at least one analyte relative to the concentration of said at least one collagen or collagen fragment.

### RELATED APPLICATION

This application is a continuation of U.S. patent application Ser. No. 12/530,029, which is a national stage filing of PCT Application No. PCT/EP2008/052730 filed Mar. 6, 2008, which claims priority of European Patent Application No. 07103684.2 filed Mar. 7, 2007, all of which are hereby incorporated herein by reference.

### TECHNICAL FIELD OF THE INVENTION

The present invention relates to a process for normalizing the concentration of analytes in a urine sample.

### BACKGROUND OF THE INVENTION

Urine samples have been used for medical laboratory examinations for a long time. Urine samples contain a variety of substances that are interesting under medical aspects, for example, degradation products of medicaments or illegal drugs, hormones, pathogens, proteins etc.

While often (only) the total protein content was recurred to for evaluating a urine sample in the past, it is recognized more and more that the composition of the proteins in urine, in particular, also contains relevant medical information.

WO 01/84140 and WO 2004/068130 describe Processes and devices for determining protein and peptide patterns in a liquid sample, for example, a urine sample.

WO 2005/024409 describes devices and processes for the quantitative evaluation of the polypeptides contained in a sample of body fluids and markers for recognizing pathological conditions. For example, it is shown how different diseases are associated with the occurrence of different polypeptide markers. It is observed that different polypeptides occur more or less frequently in certain diseases as compared to healthy patients.

WO 2006/106129 among others describes so-called amplitude markers in which two states, for example states of health and disease, are distinguished from one another, not by the occurrence, but by the amplitude, i.e., the concentration, of the markers in the sample. In order to compensate for different protein concentrations of different samples, the overall amplitude is normalized to 1 million counts after an examination of the sample by mass spectrometry in WO 2006/106129.

### SUMMARY OF THE INVENTION

However, there is still a need for more specific normalization procedures for comparing different concentrations of analytes in the sample and therefore achieve a better comparability between the reference samples and measured samples.

Surprisingly, this object can be achieved by a process for normalizing the concentration of at least one analyte in a urine sample, comprising the following steps:

a) determining the concentration of at least one analyte;
b) determining the concentration of at least one collagen or collagen fragment;
c) normalizing the concentration of said at least one analyte relative to the concentration of said at least one collagen or collagen fragment.

### BRIEF DESCRIPTION OF THE DRAWINGS

FIG. 1 depicts the regression plot of five protein standards for Example 2.

FIG. 2 depicts the normalization of the collagen fragments in Example 3.

FIG. 3 is a table listing collagen fragments and their sequences.

### DESCRIPTION OF THE INVENTION

The process according to the invention has a wide variety of applications.

First, the content of analytes, collagen or collagen fragments is determined. For the determination, for example, UV/Vis detectors, fluorescence detectors, refractive index detectors, diode array detectors, mass spectrometers, gel scanners, the staining of the proteins, for example, with Coomassie brilliant blue, silver staining, Cy2, Cy3, Cy5 or the like, or the derivatization, for example, with ITRAC (isotope tags for relative and absolute qualification) and ICAT (isotope coded affinity tagging) followed by detection of the derivatized peptides may be used.

According to the invention, collagen or collagen fragments are employed for normalizing the samples. At least one collagen or collagen fragment is identified in the sample. For this purpose, identification by migration and retention times, in particular, optionally completed by mass spectra, UV/Vis spectra, fluorescence spectra or the affinity for particular ligands, for example, specific antibodies, is suitable.

The concentration of the analyte can then be normalized to the concentration of said at least one collagen or collagen fragment, i.e., the concentrations of analytes are measured relative to the concentration of the reference collagen or collagen fragments.

Preferably, however, not one collagen or collagen fragment, but several, for example, two or three or four or five or 10, 20, 30, 40 or 50 collagens or collagen fragments are employed for normalization.

In a preferred embodiment, the analyte does not comprise any hydroxypyridinium collagen cross-links, especially no hydroxylysylpyridinoline (Pyd) and deoxypyridinoline (Dpd). Preferably, none of these compounds is employed for normalizing the concentration of another analyte either.

Preferably used collagen fragments are stated in the following Table:

| Seq. | | Swissprot | Accession | Calc. mass |
|---|---|---|---|---|
| ID No. | Protein name | name | number | [M] |
| 1 | Collagen alpha-1 (I) chain [777-787] [Homo sapiens] | CO1A1 Human | gi115269 | 1016.441 |
| 2 | Collagen alpha-1 (I) chain [546-558] [Homo sapiens] | CO1A1 Human | gi115269 | 1194.552 |
| 3 | Collagen alpha-1 (I) chain [798-810] [Homo sapiens] | CO1A1 Human | gi115269 | 1250.553 |
| 4 | Collagen alpha-1 (I) chain [546-559] [Homo sapiens] | CO1A1 Human | gi115269 | 1265.589 |
| 5 | Collagen alpha-1 (III) chain [796-808] [Homo sapiens] | CO3A1 Human | gi115306 | 1268.563 |
| 6 | Collagen alpha-1 (II) chain [511-524] [Homo sapiens] | CO2A1 Human | gi115287 | 1378.611 |
| 7 | Collagen alpha-1 (I) chain [543-558] [Homo sapiens] | CO1A1 Human | gi115269 | 1435.658 |
| 8 | Collagen alpha-1 (III) chain [642-657] [Homo sapiens] | CO3A1 Human | gi115306 | 1438.669 |
| 9 | Collagen alpha-1 (I) chain [543-558] [Homo sapiens] | CO1A1 Human | gi115269 | 1451.653 |
| 10 | Collagen alpha-1 (I) chain [542-558] [Homo sapiens] | CO1A1 Human | gi115269 | 1508.674 |
| 11 | Collagen alpha-1 (XV) chain [1093-1109] [Homo sapiens] | COFA1 Human | gi68839886 | 1523.726 |
| 12 | Collagen alpha-1 (I) chain [543-560] [Homo sapiens] | CO1A1 Human | gi115269 | 1579.711 |
| 13 | Collagen alpha-1 (III) chain [587-604] [Homo sapiens] | CO3A1 Human | gi115306 | 1623.724 |
| 14 | Collagen alpha-1 (I) chain [541-560] [Homo sapiens] | CO1A1 Human | gi115269 | 1737.781 |
| 15 | Collagen alpha-1 (III) chain [642-661] [Homo sapiens] | CO3A1 Human | gi115306 | 1834.833 |
| 16 | Collagen alpha-1 (III) chain [1174-1195] [Homo sapiens] | CO3A1 Human | gi115306 | 2025.866 |
| 17 | Collagen alpha-1 (I) chain [648-669] [Homo sapiens] | CO1A1 Human | gi115269 | 2046.949 |
| 18 | Collagen alpha-1 (I) chain [229-249] [Homo sapiens] | CO1A1 Human | gi115269 | 2063.926 |
| 19 | Collagen alpha-1 (III) chain [586-604] [Homo sapiens] | CO3A1 Human | gi115306 | 2078.925 |
| 20 | Collagen alpha-1 (I) chain [647-669] [Homo sapiens] | CO1A1 Human | gi115269 | 2087.976 |
| 21 | Collagen alpha-1 (I) chain [647-669] [Homo sapiens] | CO1A1 Human | gi115269 | 2103.971 |
| 22 | Collagen alpha-1 (I) chain [646-669] [Homo sapiens] | CO1A1 Human | gi115269 | 2159.013 |
| 23 | Collagen alpha-1 (I) chain [432-455] [Homo sapiens] | CO1A1 Human | gi115269 | 2169.977 |
| 24 | Collagen alpha-1 (I) chain [646-669] [Homo sapiens] | CO1A1 Human | gi115269 | 2175.008 |
| 25 | Collagen alpha-1 (I) chain [819-843] [Homo sapiens] | CO1A1 Human | gi115269 | 2220.988 |
| 26 | Collagen alpha-1 (I) chain [431-455] [Homo sapiens] | CO1A1 Human | gi115269 | 2226.999 |
| 27 | Collagen alpha-1 (I) chain [699-725] [Homo sapiens] | CO1A1 Human | gi115269 | 2281.979 |
| 28 | Collagen alpha-1 (I) chain [819-844] [Homo sapiens] | CO1A1 Human | gi115269 | 2292.025 |
| 29 | Collagen alpha-1 (III) chain [813-840] [Homo sapiens] | CO3A1 Human | gi115306 | 2564.149 |
| 30 | Collagen alpha-1 (III) chain [1168-1195] [Homo sapiens] | CO3A1 Human | gi115306 | 2679.202 |
| 31 | Collagen alpha-1 (III) chain [1168-1195] [Homo sapiens] | CO3A1 Human | gi115306 | 2695.197 |
| 32 | Collagen alpha-1 (III) chain [610-639] [Homo sapiens] | CO3A1 Human | gi115306 | 2742.248 |
| 33 | Collagen alpha-1 (III) chain [448-477] [Homo sapiens] | CO3A1 Human | gi115306 | 2825.270 |
| 34 | Collagen alpha-1 (I) chain [1011-1041] [Homo sapiens] | CO1A1 Human | gi115269 | 2942.299 |
| 35 | Collagen alpha-1 (I) chain [537-569] [Homo sapiens] | CO1A1 Human | gi115269 | 3011.386 |
| 36 | Collagen alpha-1 (I) chain [1011-1040] [Homo sapiens] | CO1A1 Human | gi115269 | 3013.336 |
| 37 | Collagen alpha-2 (I) chain [830-865] [Homo sapiens] | CO1A2 Human | gi82654930 | 3248.533 |
| 38 | Collagen alpha-1 (I) chain [1007-1041] [Homo sapiens] | CO1A1 Human | gi115269 | 3266.442 |
| 39 | Collagen alpha-1 (III) chain [910-948] [Homo sapiens] | CO3A1 Human | gi115306 | 3441.603 |
| 40 | Collagen alpha-1 (I) chain [630-674] [Homo sapiens] | CO1A1 Human | gi115269 | 4169.916 |
| 41 | Collagen alpha-1 (III) chain [319-366] [Homo sapiens] | CO3A1 Human | gi115306 | 4289.919 |
| 42 | Collagen alpha-1 (III) chain [319-366] [Homo sapiens] | CO3A1 Human | gi115306 | 4305.914 |

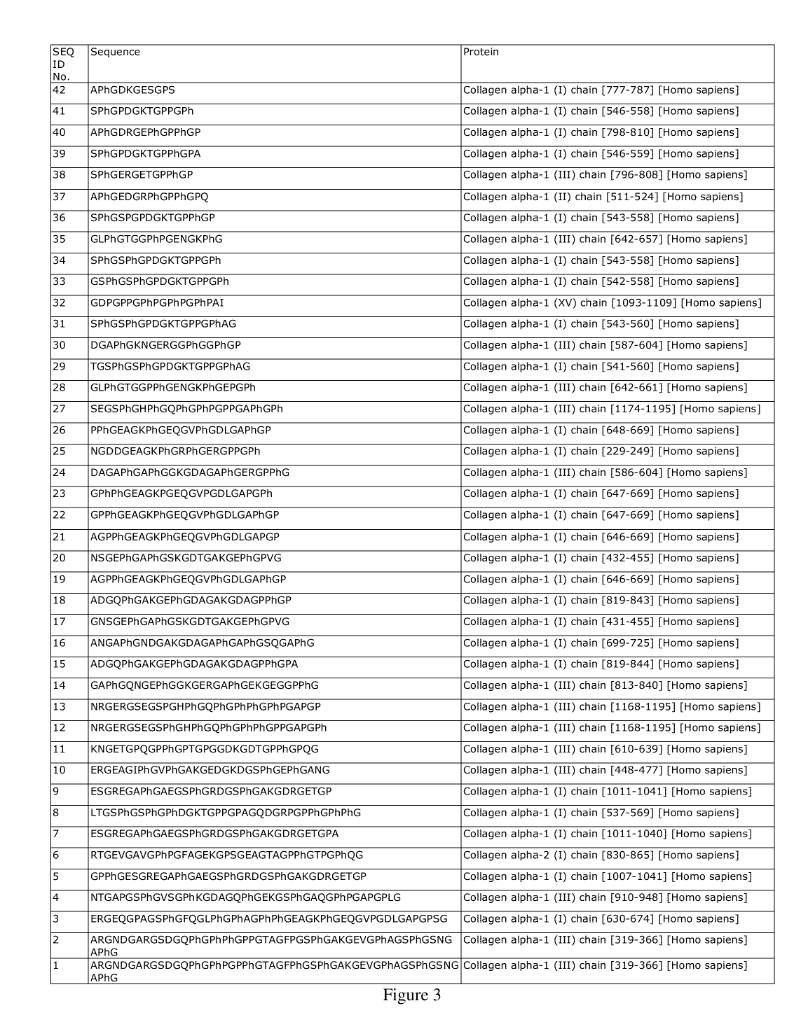

The sequences are given in FIG. 3.

The invention also relates to the use of collagen fragments for normalizing the analyte concentration of urine samples, especially with the collagen fragments as described above.

Preferred analytes whose contents are to be normalized include, in particular, proteins, peptides, lipids, metabolites, salts and other ionic compounds, carbohydrates.

The use of the process for normalizing peptide concentrations is particularly preferred.

In one embodiment, the sample is first subdivided into subsamples.

Different methods may be employed to achieve this, for example:
chromatographic methods, such as affinity chromatography, ion-exchange chromatography, reversed-phase chromatography, hydrophobic interaction chromatography, gel filtration, optionally using FPLC or HPLC;
adsorption to specific materials, such as SELDI process, ClinProt process;
electrophoretic methods, such as gel electrophoresis or capillary electrophoresis.

The invention is further illustrated by the following Examples:

### EXAMPLES

### Example 1

A urine sample was examined by means of HPLC with a downstream diode array detector (DAD). The raw date are stated in the Table. Peaks were identified by means of the retention time and spectra in the DAD. The measurement was compared with previously determined standards for these four collagen fragments, and a conversion factor was calculated for each. The average of these factors (0.80761277 in this case) was employed as a normalizing factor for all the other signals. The normalized signals are stated in the last column.

| Peak | | | Normalizing | Normalized |
|---|---|---|---|---|
| No. | Signal | Standard | factor | signal intensity |
| 1 | 126.8033157 | | | 157.0100429 |
| 2 | 146.5503894 | | | 181.4612086 |
| 3 | 3115.519875 | 4073.54509 | 0.76481782 | 3857.690207 |
| 4 | 590.1256269 | | | 730.7036846 |
| 5 | 5461.634925 | | | 6762.690148 |
| 6 | 150.3823428 | | | 186.2059991 |
| 7 | 39.1780827 | | | 48.51097474 |
| 8 | 8.5271778 | | | 10.55849798 |
| 9 | 394.6322538 | | | 488.6404331 |
| 10 | 50.0342319 | | | 61.9532451 |
| 11 | 1577.919488 | | | 1953.807005 |
| 12 | 539.3141163 | | | 667.7879996 |
| 13 | 400.4296182 | | | 495.8188292 |
| 14 | 8.8306578 | | | 10.93427212 |
| 15 | 148.7699163 | | | 184.209465 |
| 16 | 538.5410307 | | | 666.8307518 |
| 17 | 1389.860945 | | | 1720.949688 |
| 18 | 93.4206021 | | | 115.6749937 |
| 19 | 1024.846846 | 1276.56782 | 0.80281426 | 1268.982963 |
| 20 | 2344.443075 | | | 2902.929673 |
| 21 | 435.1965894 | | | 538.86789 |
| 22 | 114.2890587 | | | 141.514675 |
| 23 | 15.9397308 | | | 19.73684839 |
| 24 | 8.9629632 | | | 11.09809494 |
| 25 | 398.4975981 | | | 493.4265688 |
| 26 | 6940.8252 | 8643.25876 | 0.80303337 | 8594.248947 |
| 27 | 76.7856591 | | | 95.07732162 |
| 28 | 61.2618498 | | | 75.85547438 |
| 29 | 64.5422931 | | | 79.9173756 |
| 30 | 559.1427462 | | | 692.3401497 |
| 31 | 323.2918818 | | | 400.3055594 |
| 32 | 14.1058476 | | | 17.46610272 |
| 33 | 399.1654701 | | | 494.2535393 |
| 34 | 4612.278375 | | | 5711.002284 |
| 35 | 174.3985782 | | | 215.9433142 |
| 36 | 136.4525784 | | | 168.9579257 |
| 37 | 909.9711288 | | | 1126.741877 |
| 38 | 62.1722196 | | | 76.9827099 |
| 39 | 119.8605303 | | | 148.4133668 |
| 40 | 2922.828975 | 3399.48571 | 0.85978563 | 3619.097026 |
| 41 | 2.625588 | | | 3.251048147 |
| 42 | 52.8236532 | | | 65.40715445 |
| 43 | 178.5294621 | | | 221.0582456 |
| | | | 0.80761277 | |

### Example 2

A urine sample was separated by means of 2D electrophoresis as described in Thong-boonkerd et al. in Kidney Int 62: 1461-1469 (2002), over a pH range of from 3 to 10 and using a 10% polyacrylamide gel having a length of 22 cm. The gel was stained with Coomassie stain, and the collagens were detected in a gel prepared in parallel by means of immunological methods (Western blot). Predetermined standards for these five proteins were employed, and a linear regression performed. With this calibration formula, the further values were converted. The formula was Y=103.82x−127449. The R² value was 0.957.

| Peak | | | Normalized |
|---|---|---|---|
| No. | Signal | Standard | signal intensity |
| 1 | 4508518.988 | | 467946992.3 |
| 2 | 153935103 | | 15981414944 |
| 3 | 23582568 | | 2448214761 |
| 4 | 44251661.9 | | 4594080090 |
| 5 | 586248.746 | | 60736895.81 |
| 6 | 948640.866 | | 98360445.71 |
| 7 | 1383514.774 | | 143509054.8 |
| 8 | 122812941 | | 12750312086 |
| 9 | 2944789.572 | | 305600604.4 |
| 10 | 30550952.65 | | 3171672456 |
| 11 | 1089289.56 | 117284244.8 | 112962593.1 |
| 12 | 577112.586 | | 59788379.68 |
| 13 | 1130932.024 | | 117285913.7 |
| 14 | 913664.024 | | 94729149.97 |
| 15 | 397773.048 | | 41169348.84 |
| 16 | 6931703.714 | | 719522030.6 |
| 17 | 38342270.25 | | 3980567048 |
| 18 | 2391089.672 | | 248115480.7 |
| 19 | 2250316.422 | | 233500401.9 |
| 20 | 932726.014 | | 96708165.77 |
| 21 | 10695075.82 | | 1110235323 |
| 22 | 1438795.038 | | 149248251.8 |
| 23 | 8548926.298 | | 887422079.3 |
| 24 | 7982762.024 | | 828642904.3 |
| 25 | 519540.4375 | 58011688.21 | 53811239.22 |
| 26 | 4877082.25 | | 506211230.2 |
| 27 | 27564764.65 | | 2861646417 |
| 28 | 20622151.95 | | 2140864367 |
| 29 | 3862566.202 | | 400884174.1 |
| 30 | 219714.4865 | 23759647.58 | 22683308.99 |
| 31 | 13240495.55 | | 1374500799 |
| 32 | 6102716.5 | | 633456578 |
| 33 | 3523566.41 | | 365689215.7 |
| 34 | 1011312.708 | | 104867036.3 |
| 35 | 10681983.25 | | 1108876052 |
| 36 | 390792.574 | | 40444636.03 |
| 37 | 3552944.744 | | 368739274.3 |
| 38 | 8037397.154 | | 834315123.5 |
| 39 | 1966378.988 | | 204022017.5 |
| 40 | 89404658.25 | | 9281864171 |
| 41 | 2051887.75 | | 212899537.2 |
| 42 | 7424138.214 | | 770646580.4 |
| 43 | 32377626.4 | | 3361317724 |
| 44 | 34631829 | | 3595349038 |
| 45 | 1948609.006 | | 202177138 |
| 46 | 107055624.3 | | 11114387461 |
| 47 | 10008414.75 | | 1038946170 |
| 48 | 3575878.756 | | 371120283.4 |
| 49 | 1359764.006 | | 141043250.1 |
| 50 | 631532.685 | 52349499.17 | 65438274.36 |
| 51 | 1981878.154 | | 205631140.9 |
| 52 | 6658241.834 | | 691131218.2 |
| 53 | 8132593.25 | | 844198382.2 |
| 54 | 5853332.798 | | 607565562.1 |
| 55 | 10995087.2 | | 1141382504 |
| 56 | 3309612.762 | | 343476548 |
| 57 | 5692416.786 | | 590859261.7 |
| 58 | 3166113.976 | | 328578504 |
| 59 | 20333775.95 | | 2110925170 |
| 60 | 32900793.65 | | 3415632948 |
| 61 | 5041336.916 | | 523264149.6 |
| 62 | 2926167.976 | | 303667310.3 |
| 63 | 10445490.05 | | 1084323328 |
| 64 | 775850.282 | | 80421327.28 |
| 65 | 339214.044 | | 35089753.05 |
| 66 | 41363211.15 | | 4294201133 |
| 67 | 23456220.45 | | 2435097358 |
| 68 | 15788615 | | 1639046560 |
| 69 | 34321605.15 | | 3563141598 |
| 70 | 897196.084 | | 93019448.44 |
| 71 | 327241.543 | 37343483.31 | 33846767.99 |
| 72 | 9414104.178 | | 977244846.8 |
| 73 | 1965153.274 | | 203894763.9 |
| 74 | 45057999.65 | | 4677794075 |
| 75 | 877939.968 | | 91020278.48 |
| 76 | 3120269.268 | | 323818906.4 |
| 77 | 3584994.964 | | 372066728.2 |
| 78 | 10224568.98 | | 1061387302 |
| 79 | 735893.154 | | 76272978.25 |
| 80 | 5907069.334 | | 613144489.3 |
| 81 | 36156493.15 | | 3753639670 |
| 82 | 10877155.98 | | 1129138884 |
| 83 | 8374707.928 | | 869334728.1 |
| 84 | 16689498.2 | | 1732576254 |
| 85 | 5895062.464 | | 611897936 |
| 86 | 1706771.568 | | 177069575.2 |

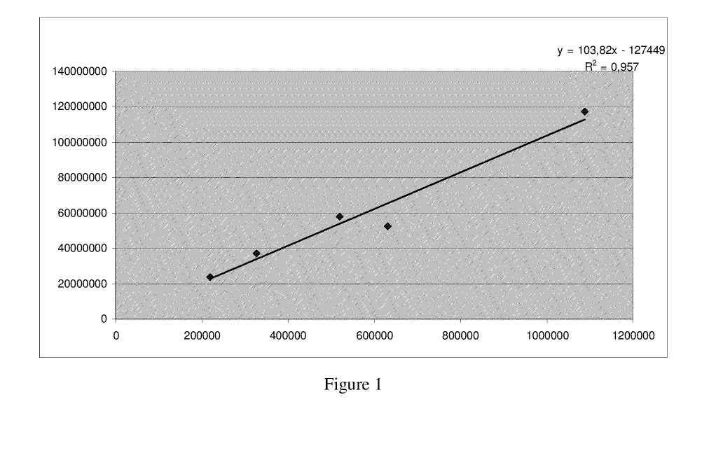

The quality of the regression is shown in FIG. 1.

### Example 3

A urine sample was examined by capillary electrophoresis coupled with mass spectrometry. Twenty-seven collagen fragments could be identified thereby, which were then compared with previously determined standards. This was followed by normalization by means of linear regression with the proviso that the straight line must go through the value zero. Corresponding measuring data and the calibration are shown in the following Table.

| | CE time | | | | Normalized |
|---|---|---|---|---|---|
| No. | (min) | Mass | Signal | Standard | signal intensity |
| 1 | 19.948 | 17266.043 | 184882.922 | | 1368.13362 |
| 2 | 19.944 | 17119.789 | 587371.188 | | 4346.54679 |
| 3 | 19.931 | 17062.977 | 240560 | | 1780.144 |
| 4 | 19.946 | 16972.203 | 352392.875 | | 2607.70728 |
| 5 | 19.908 | 16952.465 | 197797.906 | | 1463.7045 |
| 6 | 19.931 | 16918.75 | 2107725.75 | | 15597.1706 |
| 7 | 19.873 | 16806.326 | 124057.313 | | 918.024116 |
| 8 | 19.917 | 16771.715 | 1365796.63 | | 10106.895 |
| 9 | 22.387 | 10342.543 | 119401.375 | | 883.570175 |
| 10 | 20.699 | 9905.148 | 89822.563 | | 664.686966 |
| 11 | 20.727 | 9867.076 | 526946.5 | | 3899.4041 |
| 12 | 20.599 | 9626.376 | 176806.484 | | 1308.36798 |
| 13 | 20.904 | 8875.947 | 124966.711 | | 924.753661 |
| 14 | 20.927 | 8837.85 | 796843.125 | | 5896.63913 |
| 15 | 19.926 | 8578.571 | 17265.779 | | 127.766765 |
| 16 | 20.156 | 8289.725 | 286028.781 | | 2116.61298 |
| 17 | 20.078 | 8176.791 | 674227.938 | | 4989.28674 |
| 18 | 57.332 | 6788.82 | 438.533 | | 3.2451442 |
| 19 | 19.251 | 6491.179 | 95395.688 | | 705.928091 |
| 20 | 23.865 | 6329.103 | 62460.586 | | 462.208336 |
| 21 | 19.542 | 6308.096 | 23674.088 | | 175.188251 |
| 22 | 20.842 | 6237.104 | 134147.797 | | 992.693698 |
| 23 | 23.72 | 6225.298 | 67887.742 | | 502.369291 |
| 24 | 23.66 | 6208.243 | 85896.305 | | 635.632657 |
| 25 | 23.741 | 6186.583 | 288461.313 | | 2134.61372 |
| 26 | 23.643 | 6170.012 | 399146.313 | | 2953.68272 |
| 27 | 19.662 | 5861.38 | 218662.969 | | 1618.10597 |
| 28 | 22.258 | 5635.462 | 14248.398 | | 105.438145 |
| 29 | 22.266 | 5612.497 | 112633.008 | | 833.484259 |
| 30 | 22.301 | 5574.535 | 988766.188 | | 7316.86979 |
| 31 | 20.377 | 5118.564 | 18909.668 | | 139.931543 |
| 32 | 20.441 | 5096.631 | 93874.516 | | 694.671418 |
| 33 | 20.448 | 5058.56 | 250174.453 | | 1851.29095 |
| 34 | 23.737 | 5039.495 | 283460.031 | | 2097.60423 |
| 35 | 22.982 | 5038.431 | 174797.016 | | 1293.49792 |
| 36 | 20.431 | 5036.265 | 628288.625 | | 4649.33583 |
| 37 | 23.001 | 5023.362 | 43507.188 | | 321.953191 |
| 38 | 20.438 | 5020.505 | 641471.938 | | 4746.89234 |
| 39 | 23.009 | 5000.172 | 753943.563 | | 5579.18237 |
| 40 | 20.45 | 4998.592 | 2524795 | | 18683.483 |
| 41 | 20.409 | 4982.02 | 313462.719 | | 2319.62412 |
| 42 | 20.549 | 4976.683 | 1167032.75 | | 8636.04235 |
| 43 | 52.301 | 4964.782 | 358.613 | | 2.6537362 |
| 44 | 20.466 | 4960.742 | 14400323 | | 106562.39 |
| 45 | 20.358 | 4933.646 | 23247.875 | | 1652.03428 |
| 46 | 22.651 | 4876.404 | 51850.109 | | 383.690807 |
| 47 | 38.197 | 4861.141 | 494.304 | | 3.6578496 |
| 48 | 22.682 | 4838.021 | 281694.156 | | 2084.53675 |
| 49 | 22.655 | 4822.251 | 52409.906 | | 387.833304 |
| 50 | 22.683 | 4800.063 | 1058541.25 | | 7833.20525 |
| 51 | 48.573 | 4793.838 | 282.485 | | 2.090389 |
| 52 | 55.222 | 4777.634 | 374.187 | | 2.7689838 |
| 53 | 39.763 | 4774.006 | 532.92 | | 3.943608 |
| 54 | 48.826 | 4772.17 | 272.009 | | 2.0128666 |
| 55 | 57.032 | 4766.609 | 339.383 | | 2.5114342 |
| 56 | 19.62 | 4730.656 | 24256.555 | | 179.498507 |
| 57 | 37.899 | 4719.636 | 2192.44 | | 16.224056 |
| 58 | 23.902 | 4692.252 | 68363.453 | | 505.889552 |
| 59 | 54.464 | 4690.256 | 358.075 | | 2.649755 |
| 60 | 22.332 | 4671.967 | 96398.766 | | 713.350868 |
| 61 | 25.889 | 4668.661 | 32946.504 | | 243.80413 |
| 62 | 21.078 | 4656.171 | 37012.164 | | 273.890014 |
| 63 | 23.915 | 4654.204 | 252635.781 | | 1869.50478 |
| 64 | 19.366 | 4651.778 | 422.893 | | 3.1294082 |
| 65 | 25.936 | 4630.038 | 76921.227 | | 569.21708 |
| 66 | 54.567 | 4620.673 | 895.427 | | 6.6261598 |
| 67 | 23.396 | 4619.578 | 16829.051 | | 124.534977 |
| 68 | 52.701 | 4602.985 | 298.8 | | 2.21112 |
| 69 | 58.14 | 4602.554 | 423.734 | | 3.1356316 |
| 70 | 57.637 | 4594.426 | 391.032 | | 2.8936368 |
| 71 | 52.603 | 4577.292 | 340.97 | | 2.523178 |
| 72 | 20.741 | 4566.496 | 232446.344 | | 1720.10295 |
| 73 | 49.024 | 4560.303 | 642.982 | | 4.7580668 |
| 74 | 54.011 | 4559.723 | 4928.111 | | 36.4680214 |
| 75 | 18.86 | 4552.372 | 345.112 | | 2.5538288 |
| 76 | 51.846 | 4548.482 | 373.052 | | 2.7605848 |
| 77 | 26.066 | 4547.384 | 12106.5 | | 89.5881 |
| 78 | 51.365 | 4544.345 | 1851.577 | | 13.7016698 |
| 79 | 53.986 | 4543.723 | 22582.416 | | 167.109878 |
| 80 | 54.024 | 4520.931 | 4122.547 | | 30.5068478 |
| 81 | 40.87 | 4520.884 | 552.198 | | 4.0862652 |
| 82 | 25.763 | 4468.206 | 33454.656 | | 247.564454 |
| 83 | 25.839 | 4452.583 | 21824.857 | | 161.503942 |
| 84 | 48.976 | 4445.646 | 571.726 | | 4.2307724 |
| 85 | 20.501 | 4443.026 | 18179.678 | | 134.529617 |
| 86 | 27.642 | 4439.813 | 97056.883 | | 718.220934 |
| 87 | 24.137 | 4436.181 | 404617.531 | | 2994.16973 |
| 88 | 58.694 | 4420.792 | 432.338 | | 3.1993012 |
| 89 | 25.813 | 4414.273 | 15183.697 | | 112.359358 |
| 90 | 19.965 | 4410.955 | 338675.063 | | 2506.19547 |
| 91 | 20.515 | 4405.046 | 291842.344 | | 2159.63335 |
| 92 | 25.558 | 4404.052 | 42032.641 | | 311.041543 |
| 93 | 20.055 | 4391.25 | 224259.906 | | 1659.5233 |
| 94 | 59.551 | 4390.04 | 363.251 | | 2.6880574 |
| 95 | 59.07 | 4383.307 | 677.349 | | 5.0123826 |
| 96 | 20.018 | 4375.046 | 105928.648 | | 783.871995 |
| 97 | 25.571 | 4366.142 | 216682.813 | | 1603.45282 |
| 98 | 20.066 | 4353.102 | 1819283.25 | | 13462.6961 |
| 99 | 22.383 | 4349.45 | 216404.641 | | 1601.39434 |
| 100 | 25.569 | 4344.106 | 288232.313 | | 2132.91912 |
| 101 | 20.337 | 4334.652 | 45377.477 | | 335.79333 |
| 102 | 25.603 | 4328.157 | 542624 | | 4015.4176 |
| 103 | 60.458 | 4319.592 | 330.163 | | 2.4432062 |
| 104 | 27.298 | 4307.342 | 30545.982 | | 226.040267 |
| 105 | 25.614 | 4306.212 | 529838.313 | 3100.93516 | 3920.80352 |
| 106 | 18.866 | 4302.308 | 277.252 | | 2.0516648 |
| 107 | 25.618 | 4290.197 | 708976.625 | 3872.09443 | 5246.42703 |
| 108 | 25.58 | 4274.179 | 57546.574 | | 425.844648 |
| 109 | 25.607 | 4252.228 | 98721.375 | | 730.538175 |
| 110 | 23.903 | 4239.119 | 31304.807 | | 231.655572 |
| 111 | 20.202 | 4233.57 | 31109.875 | | 230.213075 |
| 112 | 25.741 | 4229.222 | 27980.471 | | 207.055485 |
| 113 | 23.93 | 4217.721 | 698545.563 | | 5169.23717 |
| 114 | 28.392 | 4208.093 | 85557.367 | | 633.124516 |
| 115 | 20.629 | 4196.917 | 227998.453 | | 1687.18855 |
| 116 | 28.393 | 4192.087 | 152065.688 | | 1125.28609 |
| 117 | 27.019 | 4180.957 | 29489.236 | | 218.220346 |
| 118 | 28.396 | 4170.166 | 176045.016 | 1291.45231 | 1302.73312 |
| 119 | 23.8 | 4160.604 | 154588.094 | | 1143.9519 |
| 120 | 22.532 | 4160.021 | 318391.75 | | 2356.09895 |
| 121 | 27.033 | 4160.011 | 69551.938 | | 514.684341 |
| 122 | 28.349 | 4154.089 | 35176.504 | | 260.30613 |
| 123 | 27.009 | 4144.035 | 62706.73 | | 464.029802 |
| 124 | 22.513 | 4144.034 | 67626.914 | | 500.439164 |
| 125 | 23.294 | 4135.927 | 40501.199 | | 299.708873 |
| 126 | 22.526 | 4122.078 | 1111014.88 | | 8221.51008 |
| 127 | 27.052 | 4121.97 | 143200.75 | | 1059.68555 |
| 128 | 26.993 | 4106.017 | 56078.742 | | 414.982691 |
| 129 | 20.242 | 4105.763 | 27811.58 | | 205.805692 |
| 130 | 28.055 | 4102.563 | 18823.371 | | 139.292945 |
| 131 | 27.022 | 4084.008 | 103050.93 | | 762.576882 |
| 132 | 28.057 | 4082.969 | 35312.172 | | 261.310073 |
| 133 | 28.103 | 4063.103 | 70996.594 | | 525.374796 |
| 134 | 20.494 | 4062.357 | 192299.219 | | 1423.01422 |
| 135 | 20.46 | 4046.127 | 45463.988 | | 336.433511 |
| 136 | 20.849 | 4044.743 | 40488.367 | | 299.613916 |
| 137 | 28.08 | 4044.394 | 166345.453 | | 1230.95635 |
| 138 | 20.276 | 4044.247 | 575594.75 | | 4259.40115 |
| 139 | 20.865 | 4028.943 | 54097.566 | | 400.321988 |
| 140 | 20.601 | 4025.685 | 273715.438 | | 2025.49424 |
| 141 | 28.152 | 4025.156 | 75278.781 | | 557.062979 |
| 142 | 25.055 | 4015.3 | 13494.786 | | 99.8614164 |
| 143 | 20.423 | 4009.552 | 46446.355 | | 343.703027 |
| 144 | 20.88 | 4007.199 | 241439.844 | | 1786.65485 |
| 145 | 28.076 | 4006.013 | 98433.141 | | 728.405243 |
| 146 | 20.719 | 3996.902 | 309862.719 | | 2292.98412 |
| 147 | 20.438 | 3986.617 | 1697006.63 | | 12557.849 |
| 148 | 20.881 | 3968.808 | 427206.25 | | 3161.32625 |
| 149 | 19.8 | 3960.04 | 129275.078 | | 956.635577 |
| 150 | 19.704 | 3913.814 | 25934.791 | | 191.917453 |
| 151 | 19.645 | 3900.239 | 29670.904 | | 219.56469 |
| 152 | 19.631 | 3877.959 | 85786.75 | | 634.82195 |
| 153 | 19.619 | 3861.996 | 59334.113 | | 439.072436 |
| 154 | 23.401 | 3860.224 | 31707.752 | | 234.637365 |
| 155 | 19.105 | 3856.541 | 110644.117 | | 818.766466 |
| 156 | 28.332 | 3839.987 | 28982.951 | | 214.473837 |
| 157 | 19.659 | 3839.739 | 105607.102 | | 781.492555 |
| 158 | 25.36 | 3832.015 | 15375.597 | | 113.779418 |
| 159 | 23.419 | 3822.288 | 21333.066 | | 157.864688 |
| 160 | 20.048 | 3814.276 | 379764.75 | | 2810.25915 |
| 161 | 28.373 | 3801.977 | 68342.133 | | 505.731784 |
| 162 | 27.677 | 3801.896 | 13939.659 | | 103.153477 |
| 163 | 27.719 | 3794.861 | 51831.453 | | 383.552752 |
| 164 | 21.453 | 3782.819 | 17585.135 | | 130.129999 |
| 165 | 31.428 | 3775.772 | 53642.129 | | 396.951755 |
| 166 | 27.744 | 3772.916 | 129047.891 | | 954.954393 |
| 167 | 21.446 | 3760.889 | 121781.781 | | 901.185179 |
| 168 | 19.224 | 3760.068 | 42083.434 | | 311.417412 |
| 169 | 21.836 | 3757.834 | 12074.36 | | 89.350264 |
| 170 | 27.7 | 3756.982 | 327179.375 | | 2421.12738 |
| 171 | 21.886 | 3741.82 | 31431.242 | | 232.591191 |
| 172 | 27.715 | 3739.881 | 56333.398 | | 416.867145 |
| 173 | 27.678 | 3735.445 | 121638.703 | | 900.126402 |
| 174 | 19.018 | 3729.583 | 19175.627 | | 141.89964 |
| 175 | 21.454 | 3722.747 | 1143063.38 | | 8458.66898 |
| 176 | 21.905 | 3720.201 | 182391.875 | | 1349.69988 |
| 177 | 27.69 | 3718.93 | 211491.453 | | 1565.03675 |
| 178 | 19.466 | 3710.955 | 28338.004 | | 209.70123 |
| 179 | 21.427 | 3706.46 | 187850.703 | | 1390.0952 |
| 180 | 21.882 | 3703.562 | 156398.141 | | 1157.34624 |
| 181 | 27.706 | 3702.282 | 83424.43 | | 617.340782 |
| 182 | 27.527 | 3699.89 | 27447.908 | | 203.114519 |
| 183 | 33.259 | 3695.777 | 61599.129 | | 455.833555 |
| 184 | 21.69 | 3686.37 | 179506.266 | | 1328.34637 |
| 185 | 31.253 | 3674.799 | 30919.297 | | 228.802798 |
| 186 | 22.842 | 3669.826 | 17117.689 | | 126.670899 |
| 187 | 18.862 | 3662.674 | 745.133 | | 5.5139842 |
| 188 | 33.247 | 3657.754 | 144639.984 | | 1070.33588 |
| 189 | 21.345 | 3630.767 | 245992.188 | | 1820.34219 |
| 190 | 19.415 | 3620.771 | 139033.516 | | 1028.84802 |
| 191 | 28.076 | 3616.437 | 45247.488 | | 334.831411 |
| 192 | 19.346 | 3605.381 | 42433.102 | | 314.004955 |
| 193 | 26.967 | 3593.676 | 72137.609 | | 533.818307 |
| 194 | 19.373 | 3582.885 | 557225.625 | | 4123.46963 |
| 195 | 27.002 | 3571.638 | 158852.625 | | 1175.50943 |
| 196 | 27.423 | 3562.87 | 16845.193 | | 124.654428 |
| 197 | 23.372 | 3559.398 | 131749.125 | | 974.943525 |
| 198 | 22.789 | 3556.364 | 63784.465 | | 472.005041 |
| 199 | 26.97 | 3555.582 | 288436.906 | | 2134.4331 |
| 200 | 26.448 | 3538.979 | 67951.078 | | 502.837977 |
| 201 | 26.967 | 3533.677 | 606124.563 | | 4485.32177 |
| 202 | 27.421 | 3531.154 | 21793.037 | | 161.268474 |
| 203 | 24.129 | 3530.806 | 68264.656 | | 505.158454 |
| 204 | 27.416 | 3524.921 | 101030.172 | | 747.623273 |
| 205 | 26.508 | 3520.84 | 46995.484 | | 347.766582 |
| 206 | 27.773 | 3518.717 | 37848.461 | | 280.078611 |
| 207 | 26.936 | 3517.813 | 749714.938 | | 5547.89054 |
| 208 | 34.02 | 3515.52 | 7304.833 | | 54.0557642 |
| 209 | 27.065 | 3511.743 | 412161.094 | | 3049.9921 |
| 210 | 27.442 | 3509.717 | 25078.984 | | 185.584482 |
| 211 | 21.028 | 3504.793 | 29887.633 | | 221.168484 |
| 212 | 19.053 | 3501.893 | 21114.822 | | 156.249683 |
| 213 | 27.027 | 3495.682 | 2833316.75 | | 20966.544 |
| 214 | 27.411 | 3493.91 | 98902.625 | | 731.879425 |
| 215 | 27.681 | 3479.778 | 17606.455 | | 130.287767 |
| 216 | 26.998 | 3479.73 | 2020814.75 | | 14954.0292 |
| 217 | 27.076 | 3473.736 | 673718 | | 4985.5132 |
| 218 | 27.432 | 3471.466 | 127158.477 | | 940.97273 |
| 219 | 19.129 | 3462.563 | 197973.266 | | 1465.00217 |
| 220 | 22.439 | 3461.026 | 35283.965 | | 261.101341 |
| 221 | 27.678 | 3457.877 | 9517.13 | | 70.426762 |
| 222 | 27.023 | 3457.775 | 3209066 | | 23747.0884 |
| 223 | 31.081 | 3456.538 | 54283.066 | | 401.694688 |
| 224 | 27.408 | 3455.346 | 234864.938 | | 1738.00054 |
| 225 | 21.428 | 3449.671 | 27877.854 | | 206.29612 |
| 226 | 26.975 | 3441.772 | 1378114.63 | 7943.47051 | 10198.0482 |
| 227 | 23.78 | 3439.69 | 46195.805 | | 341.848957 |
| 228 | 33.995 | 3438.31 | 29525.637 | | 218.489714 |
| 229 | 27.307 | 3437.3 | 69127.938 | | 511.546741 |
| 230 | 27.418 | 3433.157 | 184062.516 | | 1362.06262 |
| 231 | 25.03 | 3426.535 | 31401.645 | | 232.372173 |
| 232 | 26.945 | 3425.419 | 241693.109 | | 1788.52901 |
| 233 | 39.51 | 3422.974 | 19537.455 | | 144.577167 |
| 234 | 21.122 | 3422.818 | 189054.766 | | 1399.00527 |
| 235 | 23.945 | 3422.32 | 323563.813 | | 2394.37222 |
| 236 | 27.356 | 3416.57 | 286254.313 | | 2118.28192 |
| 237 | 27.752 | 3409.818 | 70792.961 | | 523.867911 |
| 238 | 23.959 | 3404.934 | 516072.625 | | 3818.93743 |
| 239 | 23.771 | 3402.278 | 118509.031 | | 876.966829 |
| 240 | 22.537 | 3401.814 | 94982.938 | | 702.873741 |
| 241 | 27.278 | 3397.817 | 332921.625 | | 2463.62003 |
| 242 | 32.149 | 3390.576 | 31476.922 | | 232.929223 |
| 243 | 23.818 | 3385.656 | 511551.25 | | 3785.47925 |
| 244 | 26.67 | 3385.149 | 22069.303 | | 163.312842 |
| 245 | 30.445 | 3379.519 | 15737.849 | | 116.460083 |
| 246 | 47.154 | 3378.513 | 38705.031 | | 286.417229 |
| 247 | 32.345 | 3374.638 | 87858.953 | | 650.156252 |
| 248 | 22.155 | 3374.546 | 23420.533 | | 173.311944 |
| 249 | 32.06 | 3368.627 | 127597.813 | | 944.223816 |
| 250 | 26.668 | 3363.626 | 74247.625 | | 549.432425 |
| 251 | 27.298 | 3359.786 | 207612.594 | | 1536.3332 |
| 252 | 30.446 | 3357.522 | 68808.484 | | 509.182782 |
| 253 | 32.051 | 3352.655 | 181481.938 | | 1342.96634 |
| 254 | 19.112 | 3348.489 | 28785.637 | | 213.013714 |
| 255 | 26.668 | 3347.627 | 69073.266 | | 511.142168 |
| 256 | 30.435 | 3341.531 | 95088.156 | | 703.652354 |
| 257 | 21.994 | 3341.505 | 21804.309 | | 161.351887 |
| 258 | 47.117 | 3340.88 | 42485.211 | | 314.390561 |
| 259 | 22.583 | 3338.746 | 84856.977 | | 627.94163 |
| 260 | 22.108 | 3337.336 | 52467.945 | | 388.262793 |
| 261 | 19.069 | 3332.833 | 143378.078 | | 1060.99778 |
| 262 | 32.236 | 3330.681 | 741472.813 | | 5486.89882 |
| 263 | 19.595 | 3329.771 | 22509.027 | | 166.5668 |
| 264 | 22.011 | 3325.803 | 31233.105 | | 231.124977 |
| 265 | 26.671 | 3325.558 | 349720.469 | | 2587.93147 |
| 266 | 30.442 | 3319.545 | 242232.234 | | 1792.51853 |
| 267 | 47.101 | 3319.288 | 35056.176 | | 259.415702 |
| 268 | 22.112 | 3318.936 | 56471.188 | | 417.886791 |
| 269 | 32.133 | 3314.687 | 255600.891 | | 1891.44659 |
| 270 | 23.088 | 3311.52 | 14913.211 | | 110.357761 |
| 271 | 26.667 | 3308.661 | 233464.578 | | 1727.63788 |
| 272 | 47.562 | 3305.413 | 8993.222 | | 66.5498428 |
| 273 | 22.13 | 3303.866 | 168833.016 | | 1249.36432 |
| 274 | 26.694 | 3303.689 | 52945.629 | | 391.797655 |
| 275 | 30.428 | 3303.55 | 190189.688 | | 1407.40369 |
| 276 | 23.897 | 3302.688 | 21441.1 | | 158.66414 |
| 277 | 47.167 | 3302.666 | 31385.865 | | 232.255401 |
| 278 | 22.207 | 3296.58 | 29308.854 | | 216.88552 |
| 279 | 32.299 | 3292.687 | 1206767.88 | | 8930.08228 |
| 280 | 22.855 | 3290.677 | 116553.609 | | 862.496707 |
| 281 | 22.129 | 3287.533 | 19792.754 | | 146.46638 |
| 282 | 26.675 | 3286.98 | 664343.75 | | 4916.14375 |
| 283 | 30.451 | 3281.458 | 136281.25 | | 1008.48125 |
| 284 | 23.91 | 3280.708 | 71741.43 | | 530.886582 |
| 285 | 22.104 | 3280.6 | 132426.766 | | 979.958068 |
| 286 | 27.844 | 3278.421 | 54118.781 | | 400.478979 |
| 287 | 26.669 | 3271.229 | 201540.094 | | 1491.3967 |
| 288 | 21.895 | 3268.819 | 33018.098 | | 244.333925 |
| 289 | 22.073 | 3266.64 | 409110.469 | 2799.38997 | 3027.41747 |
| 290 | 47.665 | 3266.519 | 3664.112 | | 27.1144288 |
| 291 | 26.645 | 3264.684 | 39645.684 | | 293.378062 |
| 292 | 30.445 | 3264.57 | 33225.082 | | 245.865607 |
| 293 | 32.145 | 3261.544 | 154194.859 | | 1141.04196 |
| 294 | 33.87 | 3260.577 | 9901.048 | | 73.2677552 |
| 295 | 22.173 | 3258.623 | 204077.922 | | 1510.17662 |
| 296 | 27.787 | 3256.555 | 242475 | | 1794.315 |
| 297 | 19.366 | 3253.774 | 9140 | | 67.636 |
| 298 | 25.228 | 3252.451 | 40806.734 | | 301.969832 |
| 299 | 26.638 | 3248.195 | 345009.313 | 1768.79215 | 2553.06892 |
| 300 | 21.996 | 3247.551 | 279443.219 | | 2067.87982 |
| 301 | 30.051 | 3238.459 | 73868.313 | | 546.625516 |
| 302 | 21.997 | 3231.53 | 106226.703 | | 786.077602 |
| 303 | 21.23 | 3223.641 | 53736.922 | | 397.653223 |
| 304 | 32.12 | 3222.565 | 133680.984 | | 989.239282 |
| 305 | 26.44 | 3218.542 | 24706.822 | | 182.830483 |
| 306 | 22.018 | 3209.292 | 976666.75 | | 7227.33395 |
| 307 | 19.501 | 3205.379 | 172521.531 | | 1276.65933 |
| 308 | 25.911 | 3200.625 | 30798.475 | | 227.908715 |
| 309 | 30.054 | 3200.553 | 44472.367 | | 329.095516 |
| 310 | 26.275 | 3197.269 | 120978.656 | | 895.242054 |
| 311 | 18.965 | 3195.145 | 1365.552 | | 10.1050848 |
| 312 | 21.98 | 3193.339 | 46203.789 | | 341.908039 |
| 313 | 47.564 | 3190.567 | 6346.223 | | 46.9620502 |
| 314 | 26.894 | 3187.444 | 70904.891 | | 524.696193 |
| 315 | 47.302 | 3187.001 | 26800.559 | | 198.324137 |
| 316 | 31.823 | 3183.572 | 62859.313 | | 465.158916 |
| 317 | 27.686 | 3181.527 | 130513.648 | | 965.800995 |
| 318 | 20.726 | 3180.29 | 149180.203 | | 1103.9335 |
| 319 | 26.411 | 3175.418 | 95767.344 | | 708.678346 |
| 320 | 26.942 | 3166.985 | 32730.686 | | 242.207076 |
| 321 | 21.419 | 3166.416 | 143203.063 | | 1059.70267 |
| 322 | 26.351 | 3159.36 | 255623.938 | | 1891.61714 |
| 323 | 29.385 | 3158.431 | 24115.967 | | 178.458156 |
| 324 | 20.733 | 3153.308 | 214732.906 | | 1589.0235 |
| 325 | 30.597 | 3152.559 | 13341.057 | | 98.7238218 |
| 326 | 23.226 | 3152.484 | 36119.008 | | 267.280659 |
| 327 | 26.922 | 3149.624 | 114393.813 | | 846.514216 |
| 328 | 47.176 | 3148.686 | 135798.828 | | 1004.91133 |
| 329 | 22.866 | 3148.426 | 81875.969 | | 605.882171 |
| 330 | 32.019 | 3145.601 | 228017.438 | | 1687.32904 |
| 331 | 27.665 | 3143.604 | 316068.156 | | 2338.90435 |
| 332 | 26.033 | 3139.523 | 182642.438 | | 1351.55404 |
| 333 | 60.141 | 3138.904 | 21558.727 | | 159.53458 |
| 334 | 26.459 | 3137.595 | 153225.172 | | 1133.86627 |
| 335 | 29.41 | 3137.466 | 52329.82 | | 387.240668 |
| 336 | 26.846 | 3130.813 | 68584.234 | | 507.523332 |
| 337 | 30.606 | 3130.447 | 81592.641 | | 603.785543 |
| 338 | 47.079 | 3126.892 | 51823.832 | | 383.496357 |
| 339 | 26.408 | 3121.15 | 278297.125 | | 2059.39873 |
| 340 | 26.149 | 3117.737 | 33942.793 | | 251.176668 |
| 341 | 19.395 | 3116.31 | 11625.266 | | 86.0269684 |
| 342 | 29.428 | 3115.482 | 75318.172 | | 557.354473 |
| 343 | 21.751 | 3111.371 | 83680.453 | | 619.235352 |
| 344 | 26.927 | 3108.555 | 45816.859 | | 339.044757 |
| 345 | 47.062 | 3106.709 | 11864.485 | | 87.797189 |
| 346 | 26.248 | 3101.309 | 144908.688 | | 1072.32429 |
| 347 | 56.788 | 3101.218 | 12576.415 | | 93.065471 |
| 348 | 59.981 | 3101.187 | 25661.803 | | 189.897342 |
| 349 | 29.415 | 3099.508 | 130302.75 | | 964.24035 |
| 350 | 26.467 | 3099.349 | 120340.961 | | 890.523111 |
| 351 | 38.257 | 3093.515 | 133819.891 | | 990.267193 |
| 352 | 26.873 | 3092.646 | 79548.953 | | 588.662252 |
| 353 | 25.409 | 3091.628 | 74034.063 | | 547.852066 |
| 354 | 21.782 | 3089.383 | 140512.359 | | 1039.79146 |
| 355 | 26.165 | 3086.295 | 48012.742 | | 355.294291 |
| 356 | 60.105 | 3085.019 | 7315.923 | | 54.1378302 |
| 357 | 28.833 | 3083.524 | 10016.655 | | 74.123247 |
| 358 | 45.098 | 3080.227 | 54908.891 | | 406.325793 |
| 359 | 26.28 | 3079.63 | 200529.125 | | 1483.91553 |
| 360 | 29.426 | 3077.473 | 365503.375 | | 2704.72498 |
| 361 | 19.423 | 3076.296 | 244458.344 | | 1808.99175 |
| 362 | 21.716 | 3073.391 | 19542.326 | | 144.613212 |
| 363 | 20.524 | 3064.362 | 67399.18 | | 498.753932 |
| 364 | 26.328 | 3063.327 | 269186.313 | | 1991.97872 |
| 365 | 29.434 | 3061.477 | 296489.781 | | 2194.02438 |
| 366 | 23.304 | 3060.546 | 57847.941 | | 428.074763 |
| 367 | 22.449 | 3059.408 | 131458.734 | | 972.794632 |
| 368 | 38.508 | 3055.55 | 361900.688 | | 2678.06509 |
| 369 | 21.798 | 3051.223 | 597188.688 | | 4419.19629 |
| 370 | 26.15 | 3049.373 | 185624.422 | | 1373.62072 |
| 371 | 28.742 | 3045.463 | 33384.316 | | 247.043938 |
| 372 | 22.439 | 3043.271 | 33506.316 | | 247.946738 |
| 373 | 26.25 | 3041.552 | 275291.813 | | 2037.15942 |
| 374 | 29.434 | 3039.565 | 1232005.13 | | 9116.83793 |
| 375 | 31.704 | 3039.504 | 14986.015 | | 110.896511 |
| 376 | 31.401 | 3039.5 | 17652.994 | | 130.632156 |
| 377 | 30.288 | 3038.548 | 53233.535 | | 393.928159 |
| 378 | 21.744 | 3036.442 | 318586.125 | | 2357.53733 |
| 379 | 26.1 | 3033.408 | 63530.199 | | 470.123473 |
| 380 | 29.428 | 3023.508 | 440285.75 | | 3258.11455 |
| 381 | 23.309 | 3023.197 | 35494.395 | | 262.658523 |
| 382 | 20.855 | 3022.938 | 49940.453 | | 369.559352 |
| 383 | 22.458 | 3021.485 | 264509 | | 1957.3666 |
| 384 | 38.552 | 3018.517 | 10132.168 | | 74.9780432 |
| 385 | 47.558 | 3016.717 | 14738.372 | | 109.063953 |
| 386 | 19.317 | 3015.296 | 7210.649 | | 53.3588026 |
| 387 | 21.764 | 3013.407 | 2745916.75 | | 20319.784 |
| 388 | 52.853 | 3013.224 | 6434.849 | | 47.6178826 |
| 389 | 26.165 | 3011.541 | 328700.25 | 1828.86053 | 2432.38185 |
| 390 | 53.981 | 3011.136 | 9101.616 | | 67.3519584 |
| 391 | 22.771 | 3002.372 | 31709.063 | | 234.647066 |
| 392 | 29.689 | 3001.477 | 1807418.13 | | 13374.8941 |
| 393 | 29.788 | 2999.396 | 10075.938 | | 74.5619412 |
| 394 | 21.692 | 2999.39 | 458502.219 | | 3392.91642 |
| 395 | 47.256 | 2994.527 | 99911.469 | | 739.344871 |
| 396 | 54.066 | 2994.525 | 27062.277 | | 200.26085 |
| 397 | 21.71 | 2979.845 | 137706.313 | | 1019.02672 |
| 398 | 47.277 | 2978.338 | 98216.141 | | 726.799443 |
| 399 | 25.825 | 2977.451 | 51783.652 | | 383.199025 |
| 400 | 19.356 | 2977.313 | 141777.906 | | 1049.1565 |
| 401 | 28.834 | 2976.801 | 13806.644 | | 102.169166 |
| 402 | 22.995 | 2973.765 | 71126.297 | | 526.334598 |
| 403 | 54.065 | 2973.1 | 72137.359 | | 533.816457 |
| 404 | 23.115 | 2961.255 | 24611.352 | | 182.124005 |
| 405 | 29.606 | 2959.461 | 93377.992 | | 690.997141 |
| 406 | 54.087 | 2956.985 | 34407.879 | | 254.618305 |
| 407 | 47.177 | 2956.613 | 146180.453 | | 1081.73535 |
| 408 | 47.261 | 2939.827 | 19831.465 | | 146.752841 |
| 409 | 28.882 | 2939.32 | 8665.473 | | 64.1245002 |
| 410 | 23.159 | 2939.275 | 60206.324 | | 445.526798 |
| 411 | 47.074 | 2935.554 | 119347.43 | | 883.170982 |
| 412 | 54.081 | 2935.165 | 71573.266 | | 529.642168 |
| 413 | 29.202 | 2930.452 | 119639.5 | | 885.3323 |
| 414 | 23.112 | 2923.278 | 70444.055 | | 521.286007 |
| 415 | 47.154 | 2918.618 | 78469.992 | | 580.677941 |
| 416 | 25.694 | 2917.375 | 149087.75 | | 1103.24935 |
| 417 | 47.052 | 2914.597 | 30523.621 | | 225.874795 |
| 418 | 29.136 | 2914.306 | 40968.273 | | 303.16522 |
| 419 | 29.197 | 2913.596 | 18412.203 | | 136.250302 |
| 420 | 19.329 | 2913.459 | 99422.539 | | 735.726789 |
| 421 | 23.807 | 2912.353 | 384302.219 | | 2843.83642 |
| 422 | 23.119 | 2901.3 | 312914.094 | | 2315.5643 |
| 423 | 25.911 | 2898.456 | 18701.76 | | 138.393024 |
| 424 | 38.603 | 2898.411 | 5865.952 | | 43.4080448 |
| 425 | 29.2 | 2892.487 | 466333.719 | | 3450.86952 |
| 426 | 19.686 | 2887.621 | 72285.609 | | 534.913507 |
| 427 | 23.124 | 2885.349 | 248764.953 | | 1840.86065 |
| 428 | 23.181 | 2879.347 | 44899.32 | | 332.254968 |
| 429 | 29.188 | 2876.501 | 149361.734 | | 1105.27683 |
| 430 | 20.174 | 2864.504 | 170329.094 | | 1260.4353 |
| 431 | 23.108 | 2863.359 | 1062454 | | 7862.1596 |
| 432 | 25.816 | 2861.674 | 45627.336 | | 337.642286 |
| 433 | 29.189 | 2854.531 | 504398.031 | | 3732.54543 |
| 434 | 22.467 | 2849.122 | 98682.781 | | 730.252579 |
| 435 | 23.101 | 2847.369 | 329935.469 | | 2441.52247 |
| 436 | 30.997 | 2840.951 | 29558.09 | | 218.729866 |
| 437 | 23.206 | 2840.788 | 288407.906 | | 2134.2185 |
| 438 | 45.501 | 2839.222 | 19862.145 | | 146.979873 |
| 439 | 22.887 | 2838.17 | 401500.781 | | 2971.10578 |
| 440 | 23.479 | 2837.179 | 47790.191 | | 353.647413 |
| 441 | 23.12 | 2825.369 | 2478449 | 13220.0253 | 18340.5226 |
| 442 | 47.398 | 2824.479 | 66262.844 | | 490.345046 |
| 443 | 25.837 | 2823.447 | 161446.953 | | 1194.70745 |
| 444 | 23.493 | 2823.263 | 13751.687 | | 101.762484 |
| 445 | 31.001 | 2818.979 | 87299.125 | | 646.013525 |
| 446 | 25.738 | 2818.354 | 25736.781 | | 190.452179 |
| 447 | 45.063 | 2816.103 | 114974.227 | | 850.80928 |
| 448 | 23.099 | 2808.413 | 98189.281 | | 726.600679 |
| 449 | 47.299 | 2802.496 | 96102.875 | | 711.161275 |
| 450 | 25.713 | 2802.334 | 14592.676 | | 107.985802 |
| 451 | 34.356 | 2802.334 | 85762.539 | | 634.642789 |
| 452 | 23.512 | 2799.188 | 1044906.38 | | 7732.30718 |
| 453 | 47.289 | 2786.471 | 144146.313 | | 1066.68272 |
| 454 | 29.59 | 2781.738 | 10014.258 | | 74.1055092 |
| 455 | 25.737 | 2780.371 | 61741.945 | | 456.890393 |
| 456 | 56.764 | 2772.108 | 7783.357 | | 57.5968418 |
| 457 | 54.717 | 2772.02 | 10717.143 | | 79.3068582 |
| 458 | 21.387 | 2766.811 | 49527.543 | | 366.503818 |
| 459 | 47.176 | 2764.54 | 301319.875 | | 2229.76708 |
| 460 | 34.412 | 2764.419 | 401417.969 | | 2970.49297 |
| 461 | 22.615 | 2755.244 | 63484.922 | | 469.788423 |
| 462 | 26.046 | 2754.507 | 67685.281 | | 500.871079 |
| 463 | 45.398 | 2751.163 | 21037.008 | | 155.673859 |
| 464 | 47.208 | 2747.715 | 118178.953 | | 874.524252 |
| 465 | 47.1 | 2743.596 | 170694.891 | | 1263.14219 |
| 466 | 25.731 | 2742.398 | 138049.344 | 1296.81025 | 1021.56515 |
| 467 | 19.271 | 2733.962 | 31792.85 | | 235.26709 |
| 468 | 22.636 | 2733.228 | 122354.352 | | 905.422205 |
| 469 | 47.344 | 2726.574 | 36973.457 | | 273.603582 |
| 470 | 34.503 | 2726.404 | 137639.047 | | 1018.52895 |
| 471 | 21.913 | 2720.199 | 13717.608 | | 101.510299 |
| 472 | 19.219 | 2718.289 | 12883.684 | | 95.3392616 |
| 473 | 19.051 | 2717.641 | 33752.184 | | 249.766162 |
| 474 | 22.626 | 2717.287 | 228040.188 | | 1687.49739 |
| 475 | 34.147 | 2706.346 | 28067.836 | | 207.701986 |
| 476 | 22.609 | 2700.793 | 87403.336 | | 646.784686 |
| 477 | 19.26 | 2696.284 | 111610.914 | | 825.920764 |
| 478 | 22.644 | 2695.322 | 489907.813 | 3209.57073 | 3625.31782 |
| 479 | 29.391 | 2688.188 | 62864.418 | | 465.196693 |
| 480 | 53.432 | 2685.952 | 9794.151 | | 72.4767174 |
| 481 | 21.026 | 2682.281 | 54992.879 | | 406.947305 |
| 482 | 21.953 | 2682.264 | 164811.766 | | 1219.60707 |
| 483 | 19.234 | 2680.347 | 52036.555 | | 385.070507 |
| 484 | 22.642 | 2679.325 | 348845.031 | 2843.92567 | 2581.45323 |
| 485 | 53.436 | 2669.979 | 28587.529 | | 211.547715 |
| 486 | 29.453 | 2666.015 | 134377.281 | | 994.391879 |
| 487 | 21.154 | 2663.759 | 77780.211 | | 575.573561 |
| 488 | 22.618 | 2663.307 | 45806.359 | | 338.967057 |
| 489 | 45.5 | 2663.131 | 10745.179 | | 79.5143246 |
| 490 | 45.202 | 2663.093 | 9994.085 | | 73.956229 |
| 491 | 19.308 | 2658.394 | 889974 | | 6585.8076 |
| 492 | 22.792 | 2653.835 | 10012.66 | | 74.093684 |
| 493 | 29.409 | 2649.748 | 209835.844 | | 1552.78525 |
| 494 | 53.492 | 2647.894 | 109457.719 | | 809.987121 |
| 495 | 34.123 | 2646.285 | 83952.914 | | 621.251564 |
| 496 | 21.014 | 2644.389 | 143070.844 | | 1058.72425 |
| 497 | 24.958 | 2641.325 | 9244.972 | | 68.4127928 |
| 498 | 22.26 | 2640.197 | 37788.34 | | 279.633716 |
| 499 | 28.958 | 2638.48 | 18692.865 | | 138.327201 |
| 500 | 25.376 | 2637.34 | 43694.086 | | 323.336236 |
| 501 | 47.401 | 2632.529 | 85583.328 | | 633.316627 |
| 502 | 53.456 | 2631.985 | 46383.336 | | 343.236686 |
| 503 | 23.975 | 2631.124 | 19223.193 | | 142.251628 |
| 504 | 34.021 | 2630.294 | 11402.769 | | 84.3804906 |
| 505 | 29.459 | 2628.371 | 42183.633 | | 312.158884 |
| 506 | 22.281 | 2624.209 | 24509.521 | | 181.370455 |
| 507 | 29.621 | 2622.336 | 14705.738 | | 108.822461 |
| 508 | 25.361 | 2620.846 | 14378.705 | | 106.402417 |
| 509 | 25.496 | 2620.25 | 48204.504 | | 356.71333 |
| 510 | 22.285 | 2618.226 | 25010.121 | | 185.074895 |
| 511 | 47.465 | 2615.493 | 7661.221 | | 56.6930354 |
| 512 | 47.164 | 2615.485 | 6246.06 | | 46.220844 |
| 513 | 29.331 | 2611.332 | 51577.367 | | 381.672516 |
| 514 | 47.268 | 2610.486 | 133230.797 | | 985.907898 |
| 515 | 53.423 | 2609.623 | 65116.852 | | 481.864705 |
| 516 | 34.267 | 2608.302 | 477189.5 | | 3531.2023 |
| 517 | 36.436 | 2608.217 | 49190.086 | | 364.006636 |
| 518 | 25.258 | 2606.235 | 8864.643 | | 65.5983582 |
| 519 | 22.254 | 2602.233 | 250098.953 | | 1850.73225 |
| 520 | 28.955 | 2600.859 | 42927.066 | | 317.660288 |
| 521 | 47.321 | 2594.475 | 174972.719 | | 1294.79812 |
| 522 | 23.962 | 2593.309 | 31956.934 | | 236.481312 |
| 523 | 34.191 | 2592.376 | 73723.086 | | 545.550836 |
| 524 | 29.194 | 2589.416 | 24432.801 | | 180.802727 |
| 525 | 21.055 | 2586.81 | 447437.063 | | 3311.03427 |
| 526 | 29.643 | 2584.386 | 51728.656 | | 382.792054 |
| 527 | 25.285 | 2583.339 | 57463.207 | | 425.227732 |
| 528 | 22.289 | 2580.278 | 184628.453 | | 1366.25055 |
| 529 | 25.458 | 2579.266 | 17502.498 | | 129.518485 |
| 530 | 23.288 | 2577.366 | 55801.965 | | 412.934541 |
| 531 | 45.349 | 2575.055 | 55573.074 | | 411.240748 |
| 532 | 47.193 | 2572.49 | 283804.656 | | 2100.15445 |
| 533 | 34.366 | 2570.327 | 278517.688 | | 2061.03089 |
| 534 | 53.788 | 2565.094 | 15309.151 | | 113.287717 |
| 535 | 22.285 | 2564.171 | 522868.969 | 5032.24845 | 3869.23037 |
| 536 | 25.111 | 2563.298 | 69871.031 | | 517.045629 |
| 537 | 21.035 | 2563.271 | 214366.938 | | 1586.31534 |
| 538 | 28.935 | 2560.247 | 22648.59 | | 167.599566 |
| 539 | 19.241 | 2559.267 | 287009.563 | | 2123.87077 |
| 540 | 47.221 | 2555.777 | 175942.031 | | 1301.97103 |
| 541 | 23.992 | 2554.181 | 34969.023 | | 258.77077 |
| 542 | 47.112 | 2551.558 | 249344.906 | | 1845.1523 |
| 543 | 54.133 | 2549.837 | 10977.327 | | 81.2322198 |
| 544 | 53.85 | 2548.39 | 47193.648 | | 349.232995 |
| 545 | 21.105 | 2548.131 | 191514.047 | | 1417.20395 |
| 546 | 25.467 | 2541.55 | 46554.676 | | 344.504602 |
| 547 | 22.079 | 2540.416 | 70642.961 | | 522.757911 |
| 548 | 47.061 | 2539.548 | 9106.932 | | 67.3912968 |
| 549 | 22.976 | 2538.006 | 43224.359 | | 319.860257 |
| 550 | 23.949 | 2536.124 | 11100.246 | | 82.1418204 |
| 551 | 47.048 | 2534.558 | 93213.172 | | 689.777473 |
| 552 | 53.76 | 2531.854 | 6839.3 | | 50.61082 |
| 553 | 47.021 | 2530.606 | 81025.352 | | 599.587605 |
| 554 | 20.664 | 2527.292 | 182897.938 | | 1353.44474 |
| 555 | 53.86 | 2525.836 | 124043.008 | | 917.918259 |
| 556 | 25.163 | 2525.357 | 195437.094 | | 1446.2345 |
| 557 | 24.934 | 2521.218 | 35385.203 | | 261.850502 |
| 558 | 49.205 | 2520.943 | 7070.417 | | 52.3210858 |
| 559 | 22.186 | 2518.416 | 12192.377 | | 90.2235898 |
| 560 | 23.995 | 2514.154 | 31468.1 | | 232.86394 |
| 561 | 53.893 | 2509.854 | 74708.906 | | 552.845904 |
| 562 | 53.849 | 2503.849 | 66345.75 | | 490.95855 |
| 563 | 22.127 | 2502.45 | 883793.5 | | 6540.0719 |
| 564 | 23.949 | 2498.133 | 13605.738 | | 100.682461 |
| 565 | 30.369 | 2491.037 | 55242.645 | | 408.795573 |
| 566 | 53.921 | 2487.847 | 73487.078 | | 543.804377 |
| 567 | 45.471 | 2487.024 | 20252.117 | | 149.865666 |
| 568 | 53.508 | 2483.887 | 9788.317 | | 72.4335458 |
| 569 | 55.095 | 2483.245 | 22470.289 | | 166.280139 |
| 570 | 24.966 | 2483.135 | 101523.984 | | 751.277482 |
| 571 | 50.133 | 2480.983 | 6654.337 | | 49.2420938 |
| 572 | 28.907 | 2480.171 | 48322.785 | | 357.588609 |
| 573 | 37.468 | 2479.164 | 10220.078 | | 75.6285772 |
| 574 | 29.264 | 2471.298 | 17940.619 | | 132.760581 |
| 575 | 25.284 | 2468.22 | 27459.982 | | 203.203867 |
| 576 | 53.575 | 2466.851 | 50476.148 | | 373.523495 |
| 577 | 55.082 | 2466.643 | 85574.617 | | 633.252166 |
| 578 | 28.208 | 2466.149 | 19103.148 | | 141.363295 |
| 579 | 30.326 | 2452.741 | 105720.547 | | 782.332048 |
| 580 | 25.383 | 2452.241 | 25429.555 | | 188.178707 |
| 581 | 53.558 | 2444.922 | 132386.922 | | 979.663223 |
| 582 | 55.038 | 2444.863 | 166593.141 | | 1232.78924 |
| 583 | 54.419 | 2443.898 | 14516.007 | | 107.418452 |
| 584 | 28.896 | 2442.177 | 99161.281 | | 733.793479 |
| 585 | 47.436 | 2440.325 | 82396.445 | | 609.733693 |
| 586 | 22.331 | 2439.349 | 26883.18 | | 198.935532 |
| 587 | 20.725 | 2437.156 | 67598.68 | | 500.230232 |
| 588 | 30.367 | 2430.738 | 112506.719 | | 832.549721 |
| 589 | 25.309 | 2430.18 | 9069.742 | | 67.1160908 |
| 590 | 37.568 | 2430.001 | 10098.263 | | 74.7271462 |
| 591 | 55.058 | 2428.844 | 151840.578 | | 1123.62028 |
| 592 | 53.565 | 2428.839 | 97552.461 | | 721.888211 |
| 593 | 21.874 | 2427.305 | 277859.406 | | 2056.1596 |
| 594 | 28.784 | 2422.657 | 49308.391 | | 364.882093 |
| 595 | 47.264 | 2418.405 | 102784.953 | | 760.608652 |
| 596 | 28.611 | 2418.047 | 23545.922 | | 174.239823 |
| 597 | 30.328 | 2415.783 | 100262.688 | | 741.943891 |
| 598 | 20.7 | 2415.168 | 241525.406 | | 1787.288 |
| 599 | 28.755 | 2415.065 | 27699.828 | | 204.978727 |
| 600 | 19.242 | 2414.29 | 19697.506 | | 145.761544 |
| 601 | 55.019 | 2411.833 | 8743.657 | | 64.7030618 |
| 602 | 37.119 | 2408.022 | 95515.813 | | 706.817016 |
| 603 | 24.956 | 2407.143 | 19052.658 | | 140.989669 |
| 604 | 56.103 | 2406.969 | 2105.086 | | 15.5776364 |
| 605 | 53.549 | 2406.949 | 105913 | | 783.7562 |
| 606 | 55.077 | 2406.902 | 128330.906 | | 949.648704 |
| 607 | 21.91 | 2405.312 | 22780.617 | | 168.576566 |
| 608 | 20.28 | 2404.141 | 42368.875 | | 313.529675 |
| 609 | 47.296 | 2402.426 | 168612.344 | | 1247.73135 |
| 610 | 20.557 | 2401.462 | 109291.289 | | 808.755539 |
| 611 | 28.781 | 2398.085 | 20548.543 | | 152.059218 |
| 612 | 28.626 | 2396.215 | 186626 | | 1381.0324 |
| 613 | 54.355 | 2395.78 | 17539.246 | | 129.79042 |
| 614 | 19.791 | 2395.33 | 38127.117 | | 282.140666 |
| 615 | 53.832 | 2395.108 | 25210.896 | | 186.56063 |
| 616 | 22.181 | 2393.58 | 28325.973 | | 209.6122 |
| 617 | 37.389 | 2392.069 | 32789.227 | | 242.64028 |
| 618 | 36.842 | 2392.022 | 16480.713 | | 121.957276 |
| 619 | 53.509 | 2390.96 | 7808.705 | | 57.784417 |
| 620 | 55.017 | 2390.746 | 56777.559 | | 420.153937 |
| 621 | 21.858 | 2389.367 | 1415681.5 | | 10476.0431 |
| 622 | 28.783 | 2384.878 | 67750.172 | | 501.351273 |
| 623 | 54.213 | 2381.804 | 9437.86 | | 69.840164 |
| 624 | 53.96 | 2379.819 | 13638.621 | | 100.925795 |
| 625 | 28.586 | 2379.768 | 179069.688 | | 1325.11569 |
| 626 | 20.738 | 2377.238 | 713037.375 | | 5276.47658 |
| 627 | 28.792 | 2377.105 | 243979.281 | | 1805.44668 |
| 628 | 54.034 | 2374.369 | 98473.961 | | 728.707311 |
| 629 | 38.119 | 2370.071 | 587311.313 | | 4346.10372 |
| 630 | 55.054 | 2368.829 | 45047.953 | | 333.354852 |
| 631 | 49.833 | 2368.79 | 7574.214 | | 56.0491836 |
| 632 | 21.712 | 2364.035 | 14890.036 | | 110.186266 |
| 633 | 47.251 | 2363.877 | 169502.969 | | 1254.32197 |
| 634 | 47.112 | 2359.46 | 226706.625 | | 1677.62903 |
| 635 | 54.06 | 2358.796 | 271180 | | 2006.732 |
| 636 | 28.638 | 2358.069 | 484792 | | 3587.4608 |
| 637 | 30.392 | 2356.841 | 24718.348 | | 182.915775 |
| 638 | 49.349 | 2356.439 | 49754.129 | | 368.180555 |
| 639 | 22.229 | 2355.161 | 64185.695 | | 474.974143 |
| 640 | 37.22 | 2354.086 | 71290.633 | | 527.550684 |
| 641 | 54.995 | 2351.738 | 14965.655 | | 110.745847 |
| 642 | 19.26 | 2343.309 | 31234.203 | | 231.133102 |
| 643 | 54.345 | 2342.704 | 55254.418 | | 408.882693 |
| 644 | 21.753 | 2342.053 | 57971.406 | | 428.988404 |
| 645 | 53.764 | 2341.883 | 102171.742 | | 756.070891 |
| 646 | 28.619 | 2341.757 | 395942.094 | | 2929.9715 |
| 647 | 24.143 | 2341.072 | 185665.328 | | 1373.92343 |
| 648 | 28.832 | 2339.135 | 119528.75 | | 884.51275 |
| 649 | 54.092 | 2336.821 | 849316.625 | | 6284.94303 |
| 650 | 49.582 | 2334.924 | 16711.031 | | 123.661629 |
| 651 | 49.932 | 2332.825 | 7154.382 | | 52.9424268 |
| 652 | 38.111 | 2332.118 | 724905.563 | | 5364.30117 |
| 653 | 28.429 | 2332.011 | 26272.477 | | 194.41633 |
| 654 | 49.784 | 2330.848 | 18374.775 | | 135.973335 |
| 655 | 20.777 | 2329.976 | 13845.191 | | 102.454413 |
| 656 | 20.781 | 2328.12 | 114579.438 | | 847.887841 |
| 657 | 21.695 | 2326.051 | 33955.465 | | 251.270441 |
| 658 | 28.514 | 2325.829 | 27821.584 | | 205.879722 |
| 659 | 24.109 | 2325.039 | 347883.406 | | 2574.3372 |
| 660 | 18.808 | 2323.646 | 3501.718 | | 25.9127132 |
| 661 | 21.857 | 2322.105 | 27377.75 | | 202.59535 |
| 662 | 54.036 | 2320.85 | 1445477 | | 10696.5298 |
| 663 | 21.736 | 2320.083 | 17368.453 | | 128.526552 |
| 664 | 28.756 | 2320.083 | 1394689.88 | | 10320.7051 |
| 665 | 31.151 | 2319.995 | 18706.303 | | 138.426642 |
| 666 | 50.101 | 2319.65 | 21160.838 | | 156.590201 |
| 667 | 49.596 | 2318.781 | 65589.969 | | 485.365771 |
| 668 | 53.96 | 2314.817 | 5407.608 | | 40.0162992 |
| 669 | 28.434 | 2313.129 | 21076.289 | | 155.964539 |
| 670 | 20.695 | 2312.394 | 183844.266 | | 1360.44757 |
| 671 | 45.447 | 2310.83 | 8350.151 | | 61.7911174 |
| 672 | 28.377 | 2309.901 | 32903.531 | | 243.486129 |
| 673 | 24.091 | 2309.052 | 53750.734 | | 397.755432 |
| 674 | 28.352 | 2308.815 | 65167.617 | | 482.240366 |
| 675 | 19.413 | 2306.093 | 11910.861 | | 88.1403714 |
| 676 | 47.374 | 2305.43 | 20267.41 | | 149.978834 |
| 677 | 30.479 | 2304.938 | 64574.715 | | 477.852891 |
| 678 | 54.005 | 2304.69 | 601818.875 | | 4453.45968 |
| 679 | 55.551 | 2304.254 | 20926.719 | | 154.857721 |
| 680 | 28.66 | 2304.2 | 441698.625 | | 3268.56983 |
| 681 | 21.748 | 2304.056 | 491680.813 | | 3638.43802 |
| 682 | 54.026 | 2298.86 | 2803671 | | 20747.1654 |
| 683 | 20.539 | 2298.221 | 189552.234 | | 1402.68653 |
| 684 | 30.798 | 2295.988 | 10958.861 | | 81.0955714 |
| 685 | 50.105 | 2295.884 | 10295.519 | | 76.1868406 |
| 686 | 19.179 | 2295.451 | 24928.195 | | 184.468643 |
| 687 | 28.816 | 2295.237 | 260138.016 | | 1925.02132 |
| 688 | 28.385 | 2295.094 | 55681.145 | | 412.040473 |
| 689 | 21.739 | 2288.101 | 57605.777 | | 426.28275 |
| 690 | 24.096 | 2287.057 | 1262872.75 | | 9345.25835 |
| 691 | 28.388 | 2287.048 | 297568.75 | | 2202.00875 |
| 692 | 20.5 | 2286.111 | 21349.635 | | 157.987299 |
| 693 | 24.027 | 2283.095 | 24350.102 | | 180.190755 |
| 694 | 54.068 | 2282.898 | 2728088.75 | | 20187.8568 |
| 695 | 28.732 | 2282.105 | 667956.938 | 6281.39651 | 4942.88134 |
| 696 | 21.782 | 2282.103 | 58645.039 | | 433.973289 |
| 697 | 30.387 | 2282 | 115145.461 | | 852.076411 |
| 698 | 38.199 | 2281.901 | 31642.785 | | 234.156609 |
| 699 | 38.703 | 2281.894 | 8948.516 | | 66.2190184 |
| 700 | 40.729 | 2281.884 | 69357.18 | | 513.243132 |
| 701 | 37.847 | 2281.877 | 7632.981 | | 56.4840594 |
| 702 | 49.628 | 2280.479 | 11230.125 | | 83.102925 |
| 703 | 53.207 | 2278.702 | 26455.098 | | 195.767725 |
| 704 | 28.903 | 2273.115 | 293589.719 | | 2172.56392 |
| 705 | 28.157 | 2272.659 | 27751.025 | | 205.357585 |
| 706 | 28.368 | 2271.091 | 240296.203 | | 1778.1919 |
| 707 | 23.951 | 2271.069 | 46381.387 | | 343.222264 |
| 708 | 54.563 | 2266.722 | 53080.328 | | 392.794427 |
| 709 | 53.746 | 2266.569 | 194273.734 | | 1437.62563 |
| 710 | 21.735 | 2266.113 | 834853.813 | | 6177.91822 |
| 711 | 47.142 | 2264.341 | 11589.805 | | 85.764557 |
| 712 | 25.004 | 2264.038 | 10342.722 | | 76.5361428 |
| 713 | 54.111 | 2260.906 | 3409623 | | 25231.2102 |
| 714 | 39.005 | 2259.881 | 8171.715 | | 60.470691 |
| 715 | 38.018 | 2259.88 | 89646.383 | | 663.383234 |
| 716 | 29.265 | 2257.021 | 369411.719 | | 2733.64672 |
| 717 | 28.144 | 2250.717 | 73907.656 | | 546.916654 |
| 718 | 28.424 | 2249.076 | 762669.688 | | 5643.75569 |
| 719 | 20.437 | 2249.042 | 143049.578 | | 1058.56688 |
| 720 | 47.446 | 2248.591 | 114672.375 | | 848.575575 |
| 721 | 53.175 | 2246.379 | 18363.697 | | 135.891358 |
| 722 | 54.105 | 2244.921 | 1221368.75 | | 9038.12875 |
| 723 | 53.209 | 2240.749 | 57652.742 | | 426.630291 |
| 724 | 49.832 | 2237.759 | 5953.025 | | 44.052385 |
| 725 | 28.924 | 2235.175 | 214885.766 | | 1590.15467 |
| 726 | 28.13 | 2234.935 | 156541.781 | | 1158.40918 |
| 727 | 28.391 | 2233.099 | 305448.406 | | 2260.3182 |
| 728 | 20.474 | 2232.684 | 41022.949 | | 303.569823 |
| 729 | 25.029 | 2231.969 | 11965.051 | | 88.5413774 |
| 730 | 23.853 | 2230.051 | 38128.531 | | 282.151129 |
| 731 | 25.127 | 2227.077 | 31312.469 | | 231.712271 |
| 732 | 47.253 | 2226.358 | 138834.063 | | 1027.37207 |
| 733 | 24.982 | 2226.04 | 37025.727 | | 273.99038 |
| 734 | 54.067 | 2222.955 | 1451186.5 | | 10738.7801 |
| 735 | 55.587 | 2222.909 | 18411.467 | | 136.244856 |
| 736 | 39.072 | 2221.946 | 758603.125 | | 5613.66313 |
| 737 | 33.365 | 2221.747 | 13675.914 | | 101.201764 |
| 738 | 49.924 | 2221.487 | 22766.725 | | 168.473765 |
| 739 | 28.601 | 2216.12 | 62841.969 | | 465.030571 |
| 740 | 21.813 | 2215.931 | 38498.379 | | 284.888005 |
| 741 | 28.165 | 2212.947 | 407865.344 | | 3018.20355 |
| 742 | 20.541 | 2211.332 | 175885.453 | | 1301.55235 |
| 743 | 28.425 | 2211.08 | 604871.438 | | 4476.04864 |
| 744 | 47.358 | 2210.38 | 198223.844 | | 1466.85645 |
| 745 | 24.99 | 2209.092 | 148361.516 | | 1097.87522 |
| 746 | 27.351 | 2208.986 | 43820.238 | | 324.269761 |
| 747 | 34.776 | 2208.878 | 7891.04 | | 58.393696 |
| 748 | 23.983 | 2208.045 | 1445503.75 | | 10696.7278 |
| 749 | 33.037 | 2207.824 | 20242.492 | | 149.794441 |
| 750 | 53.207 | 2207.742 | 5340.262 | | 39.5179388 |
| 751 | 29.961 | 2206.693 | 69426.469 | | 513.755871 |
| 752 | 39.013 | 2205.963 | 658740.688 | | 4874.68109 |
| 753 | 54.008 | 2203.897 | 11335.302 | | 83.8812348 |
| 754 | 53.189 | 2202.779 | 65413.605 | | 484.060677 |
| 755 | 19.293 | 2200.036 | 22179.658 | | 164.129469 |
| 756 | 55.068 | 2199.737 | 7793.02 | | 57.668348 |
| 757 | 28.127 | 2197.011 | 487901.156 | | 3610.46855 |
| 758 | 20.181 | 2194.916 | 131013.984 | | 969.503482 |
| 759 | 50.327 | 2194.728 | 41764.328 | | 309.056027 |
| 760 | 24.981 | 2193.839 | 73573.773 | | 544.44592 |
| 761 | 20.446 | 2193.108 | 20270.781 | | 150.003779 |
| 762 | 54.947 | 2192.113 | 67683.648 | | 500.858995 |
| 763 | 23.925 | 2192.015 | 526681.563 | | 3897.44357 |
| 764 | 21.89 | 2191.903 | 167538 | | 1239.7812 |
| 765 | 50.864 | 2191.723 | 32037.535 | | 237.077759 |
| 766 | 47.244 | 2188.395 | 251956.906 | | 1864.4811 |
| 767 | 32.649 | 2188.058 | 78885.688 | | 583.754091 |
| 768 | 24.014 | 2186.089 | 116699.281 | | 863.574679 |
| 769 | 29.978 | 2184.703 | 233078.734 | | 1724.78263 |
| 770 | 39.09 | 2183.992 | 1648277.63 | | 12197.2544 |
| 771 | 50.193 | 2178.759 | 33248.797 | | 246.041098 |
| 772 | 54.895 | 2176.709 | 42103.77 | | 311.567898 |
| 773 | 21.423 | 2175.984 | 37351.434 | | 276.400612 |
| 774 | 51.031 | 2175.813 | 37432.242 | | 276.998591 |
| 775 | 28.214 | 2175.135 | 523507.813 | 4308.36938 | 3873.95782 |
| 776 | 55.139 | 2174.695 | 12196.703 | | 90.2556022 |
| 777 | 47.254 | 2172.427 | 139909.953 | | 1035.33365 |
| 778 | 24.955 | 2171.845 | 392253.938 | | 2902.67914 |
| 779 | 54.95 | 2170.835 | 83249.852 | | 616.048905 |
| 780 | 23.99 | 2170.06 | 2801978.75 | 16950.086 | 20734.6428 |
| 781 | 33.051 | 2169.816 | 115270.117 | | 852.998866 |
| 782 | 28.029 | 2169.083 | 41560.289 | | 307.546139 |
| 783 | 29.942 | 2168.741 | 50702.242 | | 375.196591 |
| 784 | 38.918 | 2168 | 625043.188 | | 4625.31959 |
| 785 | 47.105 | 2167.452 | 166361.109 | | 1231.07221 |
| 786 | 23.628 | 2166.176 | 62101.336 | | 459.549886 |
| 787 | 27.877 | 2163.994 | 17963.393 | | 132.929108 |
| 788 | 19.292 | 2163.03 | 26186.1 | | 193.77714 |
| 789 | 28.177 | 2159.127 | 165196.703 | 2003.94316 | 1222.4556 |
| 790 | 21.729 | 2157.014 | 371334.938 | | 2747.87854 |
| 791 | 50.303 | 2156.445 | 98210.313 | | 726.756316 |
| 792 | 24.927 | 2156.036 | 134981.219 | | 998.861021 |
| 793 | 54.905 | 2155.728 | 215793.328 | | 1596.87063 |
| 794 | 50.291 | 2153.919 | 356736.531 | | 2639.85033 |
| 795 | 33.038 | 2153.853 | 17994.725 | | 133.160965 |
| 796 | 24.073 | 2153.761 | 687819.375 | | 5089.86338 |
| 797 | 21.431 | 2151.975 | 13115.051 | | 97.0513774 |
| 798 | 47.014 | 2150.457 | 80450.156 | | 595.331154 |
| 799 | 24.985 | 2150.074 | 124303.188 | | 919.843591 |
| 800 | 23.697 | 2148.078 | 14427.769 | | 106.765491 |
| 801 | 47.017 | 2146.51 | 33126.008 | | 245.132459 |
| 802 | 39.031 | 2146.033 | 1517131 | | 11226.7694 |
| 803 | 28.033 | 2142.049 | 116757.086 | | 864.002436 |
| 804 | 50.317 | 2140.053 | 49425.555 | | 365.749107 |
| 805 | 54.907 | 2139.687 | 131492.109 | | 973.041607 |
| 806 | 19.34 | 2139.264 | 16454.809 | | 121.765587 |
| 807 | 21.438 | 2138.028 | 170402.016 | | 1260.97492 |
| 808 | 49.775 | 2137.904 | 122028.063 | | 903.007666 |
| 809 | 51.01 | 2137.833 | 107543.789 | | 795.824039 |
| 810 | 24.98 | 2134.064 | 184060.313 | | 1362.04632 |
| 811 | 23.947 | 2133.997 | 14915.034 | | 110.371252 |
| 812 | 54.93 | 2133.748 | 448603.344 | | 3319.66475 |
| 813 | 23.791 | 2132.926 | 98156.297 | | 726.356598 |
| 814 | 33.034 | 2131.834 | 93856.258 | | 694.536309 |
| 815 | 49.73 | 2131.826 | 40604.926 | | 300.476452 |
| 816 | 19.309 | 2126.895 | 11998.439 | | 88.7884486 |
| 817 | 19.23 | 2125.959 | 13855.418 | | 102.530093 |
| 818 | 28.013 | 2125.696 | 179600.594 | | 1329.0444 |
| 819 | 29.033 | 2123.978 | 9567.942 | | 70.8027708 |
| 820 | 28.424 | 2123.872 | 38521.063 | | 285.055866 |
| 821 | 19.288 | 2123.304 | 41390.043 | | 306.286318 |
| 822 | 54.923 | 2117.754 | 676757.125 | | 5008.00273 |
| 823 | 50.182 | 2115.807 | 562227.938 | | 4160.48674 |
| 824 | 32.559 | 2113.632 | 14000.153 | | 103.601132 |
| 825 | 27.977 | 2110.026 | 15948.838 | | 118.021401 |
| 826 | 19.242 | 2109.934 | 16406.662 | | 121.409299 |
| 827 | 23.748 | 2108.995 | 14636.829 | | 108.312535 |
| 828 | 27.756 | 2106.99 | 38564.801 | | 285.379527 |
| 829 | 20.492 | 2105.879 | 76712.156 | | 567.669954 |
| 830 | 54.924 | 2101.744 | 308647.25 | | 2283.98965 |
| 831 | 21.456 | 2101.398 | 246869.797 | | 1826.8365 |
| 832 | 49.813 | 2099.871 | 64825.379 | | 479.707805 |
| 833 | 50.969 | 2099.86 | 21705.629 | | 160.621655 |
| 834 | 22.512 | 2098.949 | 30868.66 | | 228.428084 |
| 835 | 54.919 | 2095.774 | 1133086.88 | | 8384.84288 |
| 836 | 56.91 | 2094.771 | 16988.391 | | 125.714093 |
| 837 | 50.601 | 2094.588 | 22926.068 | | 169.652903 |
| 838 | 23.784 | 2093.206 | 75977.586 | | 562.234136 |
| 839 | 28.084 | 2088.017 | 171200.609 | 1269.99462 | 1266.88451 |
| 840 | 19.264 | 2087.973 | 350660.688 | | 2594.88909 |
| 841 | 23.117 | 2086.65 | 12786.285 | | 94.618509 |
| 842 | 27.791 | 2085.045 | 199885.375 | | 1479.15178 |
| 843 | 20.269 | 2081.018 | 229086.047 | | 1695.23675 |
| 844 | 54.93 | 2079.801 | 993210.188 | | 7349.75539 |
| 845 | 47.595 | 2079.318 | 19015.662 | | 140.715899 |
| 846 | 56.839 | 2078.89 | 19401.766 | | 143.573068 |
| 847 | 49.785 | 2077.893 | 115355.531 | | 853.630929 |
| 848 | 21.811 | 2077.762 | 31063.748 | | 229.871735 |
| 849 | 52.075 | 2077.73 | 6613.918 | | 48.9429932 |
| 850 | 21.41 | 2076.984 | 175348.891 | | 1297.58179 |
| 851 | 51.543 | 2071.648 | 7766.515 | | 57.472211 |
| 852 | 23.759 | 2071.002 | 133591.328 | | 988.575827 |
| 853 | 21.424 | 2070.003 | 109701.07 | | 811.787918 |
| 854 | 51.254 | 2069.625 | 61727.754 | | 456.78538 |
| 855 | 27.817 | 2069.09 | 222562.547 | | 1646.96285 |
| 856 | 20.532 | 2067.921 | 477863.719 | | 3536.19152 |
| 857 | 27.607 | 2063.948 | 71420.422 | | 528.511123 |
| 858 | 54.94 | 2063.804 | 166864.938 | | 1234.80054 |
| 859 | 55.581 | 2062.783 | 10409.057 | | 77.0270218 |
| 860 | 22.44 | 2059.045 | 31675.367 | | 234.397716 |
| 861 | 54.9 | 2057.817 | 1045255.63 | | 7734.89163 |
| 862 | 20.802 | 2056.804 | 23316.27 | | 172.540398 |
| 863 | 47.411 | 2056.32 | 114767.031 | | 849.276029 |
| 864 | 23.834 | 2056.014 | 75674.141 | | 559.988643 |
| 865 | 52.099 | 2054.689 | 8212.415 | | 60.771871 |
| 866 | 51.141 | 2052.646 | 7170.431 | | 53.0611894 |
| 867 | 23.596 | 2050.953 | 30659.434 | | 226.879812 |
| 868 | 23.173 | 2049.013 | 179035.266 | | 1324.86097 |
| 869 | 51.154 | 2047.635 | 93990.273 | | 695.52802 |
| 870 | 27.801 | 2047.175 | 411581 | 1703.37702 | 3045.6994 |
| 871 | 28.43 | 2045.938 | 76368.227 | | 565.12488 |
| 872 | 28.078 | 2041.99 | 11063.896 | | 81.8728304 |
| 873 | 54.923 | 2041.771 | 361323.031 | | 2673.79043 |
| 874 | 21.426 | 2039.213 | 401444.531 | | 2970.68953 |
| 875 | 40.128 | 2036.857 | 62552.297 | | 462.886998 |
| 876 | 40.967 | 2036.734 | 16498.773 | | 122.09092 |
| 877 | 19.377 | 2035.142 | 119280.563 | | 882.676166 |
| 878 | 20.833 | 2034.878 | 107200.859 | | 793.286357 |
| 879 | 51.167 | 2031.663 | 179833.078 | | 1330.76478 |
| 880 | 27.821 | 2031.125 | 226310.281 | | 1674.69608 |
| 881 | 49.933 | 2030.735 | 8329.54 | | 61.638596 |
| 882 | 20.301 | 2029.874 | 59304.598 | | 438.854025 |
| 883 | 27.841 | 2027.979 | 24592.67 | | 181.985758 |
| 884 | 27.615 | 2025.995 | 173888.672 | | 1286.77617 |
| 885 | 21.281 | 2023.988 | 15667.275 | | 115.937835 |
| 886 | 54.897 | 2019.862 | 339981.563 | | 2515.86357 |
| 887 | 47.368 | 2018.356 | 144909.141 | | 1072.32764 |
| 888 | 20.769 | 2017.852 | 41804.027 | | 309.3498 |
| 889 | 50.41 | 2017.309 | 23882.344 | | 176.729346 |
| 890 | 52.145 | 2016.741 | 25066.926 | | 185.495252 |
| 891 | 51.168 | 2015.674 | 15682.338 | | 116.049301 |
| 892 | 21.462 | 2014.872 | 676609.125 | | 5006.90753 |
| 893 | 23.598 | 2013.999 | 118936.234 | | 880.128132 |
| 894 | 27.425 | 2013.957 | 10481.886 | | 77.5659564 |
| 895 | 27.626 | 2009.933 | 150281.469 | | 1112.08287 |
| 896 | 51.219 | 2009.681 | 269662.813 | | 1995.50482 |
| 897 | 51.765 | 2008.693 | 8050.752 | | 59.5755648 |
| 898 | 27.027 | 2008.114 | 36968.852 | | 273.569505 |
| 899 | 49.949 | 2007.869 | 263181.844 | | 1947.54565 |
| 900 | 53.968 | 2007.765 | 23462.338 | | 173.621301 |
| 901 | 47.566 | 2003.37 | 5718.014 | | 42.3133036 |
| 902 | 20.834 | 1996.814 | 335825.125 | | 2485.10593 |
| 903 | 49.681 | 1995.723 | 5319.325 | | 39.363005 |
| 904 | 50.085 | 1995.688 | 7241.29 | | 53.585546 |
| 905 | 21.049 | 1994.926 | 32653.094 | | 241.632896 |
| 906 | 27.528 | 1993.98 | 20820.145 | | 154.069073 |
| 907 | 51.008 | 1993.068 | 187387.828 | | 1386.66993 |
| 908 | 49.882 | 1991.781 | 242254.469 | | 1792.68307 |
| 909 | 27.745 | 1989.977 | 116543.305 | | 862.420457 |
| 910 | 39.9 | 1989.431 | 174175.406 | | 1288.898 |
| 911 | 23.409 | 1987.045 | 367932.188 | | 2722.69819 |
| 912 | 27.172 | 1985.949 | 13387.146 | | 99.0648804 |
| 913 | 20.148 | 1980.776 | 40316.543 | | 298.342418 |
| 914 | 47.304 | 1980.392 | 93012.695 | | 688.293943 |
| 915 | 35.172 | 1977.731 | 80382.313 | | 594.829116 |
| 916 | 27.666 | 1975.777 | 70365.492 | | 520.704641 |
| 917 | 47.124 | 1975.433 | 105832.047 | | 783.157148 |
| 918 | 39.641 | 1972.397 | 211920.281 | | 1568.21008 |
| 919 | 21.264 | 1971.932 | 24054.641 | | 178.004343 |
| 920 | 41.424 | 1971.717 | 16212.052 | | 119.969185 |
| 921 | 40.892 | 1971.699 | 25774.264 | | 190.729554 |
| 922 | 51.25 | 1971.598 | 203815.859 | | 1508.23736 |
| 923 | 23.437 | 1971.004 | 533845.5 | | 3950.4567 |
| 924 | 41.952 | 1970.701 | 11751.374 | | 86.9601676 |
| 925 | 49.899 | 1969.877 | 510840.781 | | 3780.22178 |
| 926 | 54.004 | 1969.775 | 110049.828 | | 814.368727 |
| 927 | 52.803 | 1969.714 | 3832.406 | | 28.3598044 |
| 928 | 40.409 | 1967.647 | 566741.125 | | 4193.88433 |
| 929 | 51.49 | 1966.766 | 8687.899 | | 64.2904526 |
| 930 | 20.143 | 1964.822 | 68999.875 | | 510.599075 |
| 931 | 27.226 | 1963.76 | 22017.5 | | 162.9295 |
| 932 | 35.592 | 1961.849 | 215947.656 | | 1598.01265 |
| 933 | 20.186 | 1958.815 | 182788.188 | | 1352.63259 |
| 934 | 25.235 | 1955.974 | 34034.582 | | 251.855907 |
| 935 | 34.906 | 1955.842 | 23583.658 | | 174.519069 |
| 936 | 35.204 | 1954.875 | 23046.27 | | 170.542398 |
| 937 | 20.225 | 1954.822 | 104457.664 | | 772.986714 |
| 938 | 27.654 | 1954.071 | 25012.77 | | 185.094498 |
| 939 | 49.914 | 1953.531 | 149496.656 | | 1106.27525 |
| 940 | 23.846 | 1951.907 | 11188.573 | | 82.7954402 |
| 941 | 40.048 | 1951.671 | 405308.781 | | 2999.28498 |
| 942 | 49.519 | 1951.462 | 32885.328 | | 243.351427 |
| 943 | 28.011 | 1950.926 | 80130.508 | | 592.965759 |
| 944 | 29.888 | 1950.847 | 95179.586 | | 704.328936 |
| 945 | 21.331 | 1949.954 | 15990.713 | | 118.331276 |
| 946 | 19.872 | 1949.895 | 8138.52 | | 60.225048 |
| 947 | 23.457 | 1949.102 | 3147247 | | 23289.6278 |
| 948 | 27.164 | 1947.721 | 65290.855 | | 483.152327 |
| 949 | 35.176 | 1945.87 | 48158.836 | | 356.375386 |
| 950 | 51.644 | 1945.533 | 5578.079 | | 41.2777846 |
| 951 | 28.503 | 1945.117 | 26344.092 | | 194.946281 |
| 952 | 20.19 | 1942.355 | 176231.625 | | 1304.11403 |
| 953 | 36.009 | 1939.849 | 754929.75 | | 5586.48015 |
| 954 | 21.083 | 1939.728 | 83881.531 | | 620.723329 |
| 955 | 27.622 | 1937.032 | 122677.93 | | 907.816682 |
| 956 | 20.174 | 1935.815 | 14819.995 | | 109.667963 |
| 957 | 55.67 | 1935.509 | 1866.308 | | 13.8106792 |
| 958 | 19.929 | 1934.88 | 185292.125 | | 1371.16173 |
| 959 | 27.6 | 1933.946 | 21970.883 | | 162.584534 |
| 960 | 21.312 | 1933.467 | 120517.273 | | 891.82782 |
| 961 | 23.44 | 1933.132 | 1264035.38 | | 9353.86178 |
| 962 | 49.882 | 1931.835 | 169525.344 | | 1254.48755 |
| 963 | 53.987 | 1931.539 | 7912.462 | | 58.5522188 |
| 964 | 32.897 | 1930.835 | 60372.492 | | 446.756441 |
| 965 | 40.453 | 1929.642 | 1351748.75 | | 10002.9408 |
| 966 | 49.985 | 1928.736 | 30871.664 | | 228.450314 |
| 967 | 19.413 | 1928.014 | 24680.447 | | 182.635308 |
| 968 | 22.284 | 1925.639 | 28804.176 | | 213.150902 |
| 969 | 36.02 | 1923.804 | 788593 | | 5835.5882 |
| 970 | 32.585 | 1923.744 | 18391.229 | | 136.095095 |
| 971 | 51.474 | 1923.542 | 22426.631 | | 165.957069 |
| 972 | 21.155 | 1921.892 | 25471.947 | | 188.492408 |
| 973 | 20.197 | 1920.513 | 484588.406 | | 3585.9542 |
| 974 | 35.145 | 1917.819 | 151947.547 | | 1124.41185 |
| 975 | 20.275 | 1916.865 | 592958 | | 4387.8892 |
| 976 | 32.944 | 1914.85 | 53011.285 | | 392.283509 |
| 977 | 39.532 | 1913.055 | 359803.531 | | 2662.54613 |
| 978 | 27.873 | 1912.956 | 171374.188 | | 1268.16899 |
| 979 | 49.886 | 1912.77 | 90604.273 | | 670.47162 |
| 980 | 23.536 | 1911.131 | 16659075 | | 123277.155 |
| 981 | 19.165 | 1909.169 | 16568.082 | | 122.603807 |
| 982 | 35.112 | 1907.913 | 132959.188 | | 983.897991 |
| 983 | 51.45 | 1907.405 | 55725.84 | | 412.371216 |
| 984 | 20.208 | 1904.837 | 417636.094 | | 3090.5071 |
| 985 | 19.974 | 1901.937 | 12937.851 | | 95.7400974 |
| 986 | 36.035 | 1901.931 | 1493067.88 | | 11048.7023 |
| 987 | 27.44 | 1901.067 | 35484.109 | | 262.582407 |
| 988 | 21.174 | 1898.892 | 49621.984 | | 367.202682 |
| 989 | 26.836 | 1897.634 | 51880.426 | | 383.915152 |
| 990 | 22.569 | 1895.801 | 31370.744 | | 232.143506 |
| 991 | 50.839 | 1895.655 | 14071.792 | | 104.131261 |
| 992 | 19.364 | 1893.904 | 10081.896 | | 74.6060304 |
| 993 | 20.096 | 1893.708 | 16126.631 | | 119.337069 |
| 994 | 23.44 | 1893.144 | 138012.906 | | 1021.2955 |
| 995 | 33.628 | 1892.875 | 666429.375 | | 4931.57738 |
| 996 | 40.178 | 1891.728 | 965003.063 | | 7141.02267 |
| 997 | 23.747 | 1890.939 | 199265.828 | | 1474.56713 |
| 998 | 28.18 | 1889.913 | 10303.565 | | 76.246381 |
| 999 | 51.81 | 1886.097 | 32181.391 | | 238.142293 |
| 1000 | 49.855 | 1886.017 | 9415.063 | | 69.6714662 |
| 1001 | 19.928 | 1885.96 | 15694.74 | | 116.141076 |
| 1002 | 35.93 | 1885.943 | 758995.75 | | 5616.56855 |
| 1003 | 51.337 | 1885.571 | 114489.086 | | 847.219236 |
| 1004 | 20.18 | 1882.925 | 4423241 | | 32731.9834 |
| 1005 | 29.37 | 1882.543 | 68798.625 | | 509.109825 |
| 1006 | 35.135 | 1879.95 | 58723.824 | | 434.556298 |
| 1007 | 22.494 | 1879.257 | 20705.602 | | 153.221455 |
| 1008 | 34.349 | 1878.875 | 33668.203 | | 249.144702 |
| 1009 | 22.591 | 1876.914 | 37062.238 | | 274.260561 |
| 1010 | 21.725 | 1876.902 | 297599.313 | 1550.63508 | 2202.23492 |
| 1011 | 32.986 | 1876.876 | 128455.727 | | 950.57238 |
| 1012 | 55.924 | 1875.624 | 2100.854 | | 15.5463196 |
| 1013 | 26.625 | 1875.501 | 105028.836 | | 777.213386 |
| 1014 | 27.296 | 1874.767 | 67852.57 | | 502.109018 |
| 1015 | 26.891 | 1873.891 | 93489.258 | | 691.820509 |
| 1016 | 21.431 | 1872.932 | 23245.711 | | 172.018261 |
| 1017 | 19.83 | 1872.86 | 146425.875 | | 1083.55148 |
| 1018 | 51.282 | 1869.592 | 141043.234 | | 1043.71993 |
| 1019 | 20.63 | 1867.838 | 31031.188 | | 229.630791 |
| 1020 | 27.992 | 1864.675 | 33520.336 | | 248.050486 |
| 1021 | 47.391 | 1864.278 | 57918.516 | | 428.597018 |
| 1022 | 20.099 | 1864.101 | 137271.891 | | 1015.81199 |
| 1023 | 35.869 | 1863.967 | 524659.438 | | 3882.47984 |
| 1024 | 27.239 | 1863.873 | 52788.363 | | 390.633886 |
| 1025 | 20.031 | 1863.141 | 139697.438 | | 1033.76104 |
| 1026 | 19.87 | 1860.926 | 13068.771 | | 96.7089054 |
| 1027 | 21.157 | 1860.926 | 81917.836 | | 606.191986 |
| 1028 | 26.764 | 1860.34 | 197601.531 | | 1462.25133 |
| 1029 | 27.282 | 1859.5 | 48407.289 | | 358.213939 |
| 1030 | 23.006 | 1859.03 | 193516.188 | | 1432.01979 |
| 1031 | 51.461 | 1858.652 | 43128.02 | | 319.147348 |
| 1032 | 22.605 | 1857.839 | 203838.828 | | 1508.40733 |
| 1033 | 19.267 | 1855.928 | 13138.126 | | 97.2221324 |
| 1034 | 20.105 | 1855.764 | 131766.063 | | 975.068866 |
| 1035 | 33.577 | 1854.908 | 733231.875 | | 5425.91588 |
| 1036 | 26.818 | 1851.8 | 200711.625 | | 1485.26603 |
| 1037 | 60.637 | 1848.46 | 6038.243 | | 44.6829982 |
| 1038 | 19.998 | 1848.263 | 99688.43 | | 737.694382 |
| 1039 | 27.542 | 1847.933 | 53558.961 | | 396.336311 |
| 1040 | 51.445 | 1847.586 | 194541.844 | | 1439.60965 |
| 1041 | 50.289 | 1846.06 | 144120.094 | | 1066.4887 |
| 1042 | 20.658 | 1845.847 | 131101.281 | | 970.149479 |
| 1043 | 26.69 | 1844.668 | 50529.566 | | 373.918788 |
| 1044 | 51.19 | 1844.662 | 11750.058 | | 86.9504292 |
| 1045 | 36.173 | 1843.622 | 40933.469 | | 302.907671 |
| 1046 | 50.239 | 1842.658 | 50739.762 | | 375.474239 |
| 1047 | 51.616 | 1842.648 | 61330.453 | | 453.845352 |
| 1048 | 22.565 | 1841.844 | 52639.223 | | 389.53025 |
| 1049 | 20.14 | 1839.786 | 104989.461 | | 776.922011 |
| 1050 | 26.58 | 1837.73 | 168274.531 | | 1245.23153 |
| 1051 | 27.295 | 1836.888 | 154800.984 | | 1145.52728 |
| 1052 | 19.861 | 1835.81 | 239974.203 | | 1775.8091 |
| 1053 | 26.859 | 1835.179 | 177662.859 | 3450.55729 | 1314.70516 |
| 1054 | 22.947 | 1834.847 | 112151.383 | | 829.920234 |
| 1055 | 22.831 | 1832.844 | 128282.289 | | 949.288939 |
| 1056 | 20.82 | 1831.312 | 39760.145 | | 294.225073 |
| 1057 | 51.429 | 1831.23 | 74989.969 | | 554.925771 |
| 1058 | 20.578 | 1829.857 | 18786.881 | | 139.022919 |
| 1059 | 50.43 | 1829.681 | 33509.582 | | 247.970907 |
| 1060 | 33.814 | 1829.529 | 37306.023 | | 276.06457 |
| 1061 | 49.958 | 1828.949 | 54472.137 | | 403.093814 |
| 1062 | 28.024 | 1827.115 | 55237.465 | | 408.757241 |
| 1063 | 50.335 | 1826.663 | 44625.695 | | 330.230143 |
| 1064 | 51.638 | 1826.652 | 57917.66 | | 428.590684 |
| 1065 | 47.405 | 1826.309 | 86090.477 | | 637.06953 |
| 1066 | 27.216 | 1825.887 | 48555.508 | | 359.310759 |
| 1067 | 20.099 | 1825.86 | 338040.688 | | 2501.50109 |
| 1068 | 23.404 | 1824.068 | 54327.395 | | 402.022723 |
| 1069 | 55.496 | 1823.55 | 1463.899 | | 10.8328526 |
| 1070 | 26.708 | 1822.845 | 185135.641 | | 1370.00374 |
| 1071 | 27.294 | 1821.908 | 36764.754 | | 272.05918 |
| 1072 | 26.419 | 1821.874 | 13089.423 | | 96.8617302 |
| 1073 | 51.474 | 1820.716 | 52165.934 | | 386.027912 |
| 1074 | 22.606 | 1819.804 | 580688.75 | | 4297.09675 |
| 1075 | 20.154 | 1817.761 | 1081253.88 | | 8001.27868 |
| 1076 | 37.543 | 1817.747 | 40002.078 | | 296.015377 |
| 1077 | 27.079 | 1813.82 | 933491.563 | | 6907.83757 |
| 1078 | 22.791 | 1812.882 | 126053.18 | | 932.793532 |
| 1079 | 60.619 | 1810.514 | 9529.108 | | 70.5153992 |
| 1080 | 27.679 | 1809.961 | 61372.824 | | 454.158898 |
| 1081 | 51.36 | 1809.662 | 166998.938 | | 1235.79214 |
| 1082 | 25.419 | 1808.785 | 99647.727 | | 737.39318 |
| 1083 | 32.362 | 1807.95 | 28583.354 | | 211.51682 |
| 1084 | 20.635 | 1807.641 | 329490.406 | | 2438.229 |
| 1085 | 22.31 | 1806.883 | 44412.402 | | 328.651775 |
| 1086 | 40.188 | 1805.588 | 418072.781 | | 3093.73858 |
| 1087 | 50.551 | 1804.673 | 858333.75 | | 6351.66975 |
| 1088 | 54.902 | 1804.666 | 19508.908 | | 144.365919 |
| 1089 | 47.258 | 1804.327 | 91234.57 | | 675.135818 |
| 1090 | 26.77 | 1803.88 | 266848.438 | | 1974.67844 |
| 1091 | 32.344 | 1801.343 | 78405.297 | | 580.199198 |
| 1092 | 30.974 | 1800.629 | 22093.713 | | 163.493476 |
| 1093 | 19.034 | 1800.052 | 117200.094 | | 867.280696 |
| 1094 | 20.101 | 1799.824 | 27975.473 | | 207.0185 |
| 1095 | 27.283 | 1798.711 | 106091.477 | | 785.07693 |
| 1096 | 26.789 | 1796.84 | 115289.117 | | 853.139466 |
| 1097 | 22.952 | 1794.908 | 403840.938 | | 2988.42294 |
| 1098 | 27.655 | 1793.973 | 137558.906 | | 1017.9359 |
| 1099 | 20.703 | 1793.821 | 42362.82 | | 313.484868 |
| 1100 | 26.926 | 1792.934 | 245127.938 | | 1813.94674 |
| 1101 | 25.423 | 1792.81 | 76797.188 | | 568.299191 |
| 1102 | 19.261 | 1790.148 | 27753.096 | | 205.37291 |
| 1103 | 26.987 | 1789.851 | 25226.754 | | 186.67798 |
| 1104 | 38.699 | 1789.61 | 59189.73 | | 438.004002 |
| 1105 | 26.216 | 1788.907 | 15748.181 | | 116.536539 |
| 1106 | 50.525 | 1788.693 | 893111.313 | | 6609.02372 |
| 1107 | 47.617 | 1788.321 | 6225.253 | | 46.0668722 |
| 1108 | 26.718 | 1787.886 | 220469.656 | | 1631.47545 |
| 1109 | 32.412 | 1785.707 | 138173.328 | | 1022.48263 |
| 1110 | 47.08 | 1783.395 | 13617.989 | | 100.773119 |
| 1111 | 24.05 | 1782.919 | 130107.336 | | 962.794286 |
| 1112 | 26.719 | 1779.838 | 13206.108 | | 97.7251992 |
| 1113 | 26.461 | 1778.925 | 77194.57 | | 571.239818 |
| 1114 | 19.165 | 1778.824 | 6165.233 | | 45.6227242 |
| 1115 | 19.281 | 1777.115 | 29204.076 | | 216.110162 |
| 1116 | 26.899 | 1775.901 | 368163.375 | | 2724.40898 |
| 1117 | 22.755 | 1775.826 | 568114.875 | | 4204.05008 |
| 1118 | 50.249 | 1772.72 | 125865.164 | | 931.402214 |
| 1119 | 51.599 | 1772.686 | 30238.418 | | 223.764293 |
| 1120 | 19.906 | 1772.576 | 13414.021 | | 99.2637554 |
| 1121 | 26.14 | 1771.789 | 20342.643 | | 150.535558 |
| 1122 | 60.659 | 1771.538 | 4335.641 | | 32.0837434 |
| 1123 | 25.395 | 1770.678 | 364467.063 | | 2697.05627 |
| 1124 | 32.44 | 1769.84 | 87244.375 | | 645.608375 |
| 1125 | 26.406 | 1768.832 | 73680.18 | | 545.233332 |
| 1126 | 27.285 | 1768 | 51965.754 | | 384.54658 |
| 1127 | 40.282 | 1767.639 | 1154890.88 | | 8546.19248 |
| 1128 | 23.421 | 1767.108 | 56689.273 | | 419.50062 |
| 1129 | 22.915 | 1767.084 | 339444.344 | | 2511.88815 |
| 1130 | 50.388 | 1766.72 | 1244047.25 | | 9205.94965 |
| 1131 | 54.846 | 1766.713 | 18705.223 | | 138.41865 |
| 1132 | 46.966 | 1766.364 | 27324.01 | | 202.197674 |
| 1133 | 26.826 | 1766.124 | 676256.625 | | 5004.29903 |
| 1134 | 27.6 | 1765.883 | 24261.887 | | 179.537964 |
| 1135 | 19.883 | 1764.796 | 43908.137 | | 324.920214 |
| 1136 | 30.95 | 1763.615 | 10578.803 | | 78.2831422 |
| 1137 | 32.353 | 1763.538 | 197962.688 | | 1464.92389 |
| 1138 | 52.236 | 1762.263 | 24308.041 | | 179.879503 |
| 1139 | 19.154 | 1761.794 | 275627.063 | | 2039.64027 |
| 1140 | 32.005 | 1761.361 | 33788.863 | | 250.037586 |
| 1141 | 26.342 | 1760.756 | 28904.85 | | 213.89589 |
| 1142 | 22.781 | 1759.833 | 111226.188 | | 823.073791 |
| 1143 | 26.285 | 1756.792 | 65174.434 | | 482.290812 |
| 1144 | 26.977 | 1755.048 | 485130.938 | | 3589.96894 |
| 1145 | 25.433 | 1754.849 | 154997.719 | | 1146.98312 |
| 1146 | 19.568 | 1752.837 | 9993.905 | | 73.954897 |
| 1147 | 26.365 | 1752.747 | 8704.931 | | 64.4164894 |
| 1148 | 34.142 | 1752.717 | 5691.779 | | 42.1191646 |
| 1149 | 38.509 | 1751.662 | 40417.168 | | 299.087043 |
| 1150 | 51.038 | 1751.619 | 21932.201 | | 162.298287 |
| 1151 | 51.655 | 1750.712 | 48846.887 | | 361.466964 |
| 1152 | 50.127 | 1750.711 | 321034.5 | | 2375.6553 |
| 1153 | 22.842 | 1750.641 | 298645.875 | | 2209.97948 |
| 1154 | 26.646 | 1749.91 | 151839.859 | | 1123.61496 |
| 1155 | 19.696 | 1749.416 | 18542.609 | | 137.215307 |
| 1156 | 47.314 | 1749.411 | 3008.298 | | 22.2614052 |
| 1157 | 27.975 | 1748.147 | 81581.477 | | 603.70293 |
| 1158 | 32.363 | 1747.731 | 89702.695 | | 663.799943 |
| 1159 | 55.946 | 1747.592 | 6465.131 | | 47.8419694 |
| 1160 | 26.449 | 1746.84 | 61283.23 | | 453.495902 |
| 1161 | 35.783 | 1745.692 | 9288.554 | | 68.7352996 |
| 1162 | 56.832 | 1745.568 | 6267.448 | | 46.3791152 |
| 1163 | 36.089 | 1745.067 | 41401.465 | | 306.370841 |
| 1164 | 22.138 | 1744.813 | 35727.973 | | 264.387 |
| 1165 | 36.42 | 1744.373 | 27876.617 | | 206.286966 |
| 1166 | 26.218 | 1740.802 | 26704.641 | | 197.614343 |
| 1167 | 20.752 | 1740.022 | 166723.391 | | 1233.75309 |
| 1168 | 23.583 | 1738.825 | 94085.523 | | 696.23287 |
| 1169 | 26.387 | 1738.795 | 81945.344 | | 606.395546 |
| 1170 | 26.797 | 1737.895 | 254024.781 | 2608.25236 | 1879.78338 |
| 1171 | 22.79 | 1737.77 | 751127.625 | | 5558.34443 |
| 1172 | 26.66 | 1736.361 | 112063.227 | | 829.26788 |
| 1173 | 19.845 | 1734.781 | 21712.186 | | 160.670176 |
| 1174 | 21.937 | 1734.705 | 26412.207 | | 195.450332 |
| 1175 | 26.121 | 1733.55 | 44750.543 | | 331.154018 |
| 1176 | 25.448 | 1732.885 | 675405.813 | | 4998.00302 |
| 1177 | 26.799 | 1731.178 | 590140.125 | | 4367.03693 |
| 1178 | 51.061 | 1730.476 | 53972.723 | | 399.39815 |
| 1179 | 40.062 | 1729.677 | 777807.625 | | 5755.77643 |
| 1180 | 50.108 | 1728.761 | 243423.203 | | 1801.3317 |
| 1181 | 51.671 | 1728.713 | 12968.45 | | 95.96653 |
| 1182 | 33.569 | 1727.797 | 336655.563 | | 2491.25117 |
| 1183 | 32.353 | 1725.586 | 166671.5 | | 1233.3691 |
| 1184 | 18.942 | 1724.448 | 9412.43 | | 69.651982 |
| 1185 | 52.194 | 1724.241 | 44839.199 | | 331.810073 |
| 1186 | 31.985 | 1723.47 | 62237.195 | | 460.555243 |
| 1187 | 26.609 | 1722.557 | 453472.031 | | 3355.69303 |
| 1188 | 19.582 | 1720.78 | 74309.211 | | 549.888161 |
| 1189 | 27.777 | 1719.806 | 8670.013 | | 64.1580962 |
| 1190 | 26.311 | 1718.82 | 258650.984 | | 1914.01728 |
| 1191 | 22.038 | 1718.203 | 26217.236 | | 194.007546 |
| 1192 | 22.806 | 1717.892 | 71530.109 | | 529.322807 |
| 1193 | 22.835 | 1717.027 | 79037.438 | | 584.877041 |
| 1194 | 25.359 | 1716.873 | 78706.383 | | 582.427234 |
| 1195 | 20.092 | 1716.75 | 98706.563 | | 730.428566 |
| 1196 | 22.344 | 1716.589 | 114901.781 | | 850.273179 |
| 1197 | 56.213 | 1715.625 | 8737.123 | | 64.6547102 |
| 1198 | 51.077 | 1714.783 | 12487.07 | | 92.404318 |
| 1199 | 26.44 | 1712.803 | 97550.773 | | 721.87572 |
| 1200 | 21.961 | 1712.729 | 167742.406 | | 1241.2938 |
| 1201 | 33.485 | 1711.815 | 325279.094 | | 2407.0653 |
| 1202 | 19.769 | 1709.765 | 8169.03 | | 60.450822 |
| 1203 | 26.903 | 1708.88 | 616257 | | 4560.3018 |
| 1204 | 37.1 | 1707.723 | 573278.438 | | 4242.26044 |
| 1205 | 22.128 | 1706.846 | 147145.969 | | 1088.88017 |
| 1206 | 51.017 | 1706.819 | 8774.919 | | 64.9344006 |
| 1207 | 33.49 | 1705.824 | 107182.141 | | 793.147843 |
| 1208 | 26.213 | 1702.549 | 107273.141 | | 793.821243 |
| 1209 | 19.719 | 1701.773 | 10910.079 | | 80.7345846 |
| 1210 | 19.004 | 1701.117 | 286517 | | 2120.2258 |
| 1211 | 26.439 | 1700.815 | 121645.578 | | 900.177277 |
| 1212 | 22.605 | 1700.526 | 452406.844 | | 3347.81065 |
| 1213 | 31.892 | 1698.717 | 236323.828 | | 1748.79633 |
| 1214 | 26.019 | 1697.79 | 32841.801 | | 243.029327 |
| 1215 | 26.766 | 1697.722 | 293863.219 | | 2174.58782 |
| 1216 | 26.392 | 1696.803 | 58345.828 | | 431.759127 |
| 1217 | 22.005 | 1696.517 | 226208.672 | | 1673.94417 |
| 1218 | 33.389 | 1695.815 | 116949.117 | | 865.423466 |
| 1219 | 22.263 | 1693.709 | 34169.855 | | 252.856927 |
| 1220 | 20.506 | 1693.689 | 39384.203 | | 291.443102 |
| 1221 | 22.614 | 1693.346 | 26959.088 | | 199.497251 |
| 1222 | 26.76 | 1692.798 | 553402.438 | | 4095.17804 |
| 1223 | 51.073 | 1692.162 | 64894.574 | | 480.219848 |
| 1224 | 36.441 | 1691.753 | 474631.719 | | 3512.27472 |
| 1225 | 38.476 | 1691.74 | 71431.195 | | 528.590843 |
| 1226 | 33.701 | 1689.84 | 967976.563 | | 7163.02657 |
| 1227 | 20.188 | 1688.745 | 58794.035 | | 435.075859 |
| 1228 | 31.275 | 1688.712 | 23481.848 | | 173.765675 |
| 1229 | 22.111 | 1687.759 | 52805.07 | | 390.757518 |
| 1230 | 19.519 | 1687.737 | 8106.24 | | 59.986176 |
| 1231 | 32.032 | 1687.441 | 57712.715 | | 427.074091 |
| 1232 | 27.427 | 1686.789 | 19094.982 | | 141.302867 |
| 1233 | 26.916 | 1686.781 | 15861.432 | | 117.374597 |
| 1234 | 30.394 | 1686.748 | 8055.863 | | 59.6133862 |
| 1235 | 52.123 | 1686.612 | 18216.238 | | 134.800161 |
| 1236 | 20.925 | 1685.791 | 14135.824 | | 104.605098 |
| 1237 | 26.848 | 1684.778 | 1528617.38 | | 11311.7686 |
| 1238 | 51.06 | 1683.539 | 7311.187 | | 54.1027838 |
| 1239 | 56.025 | 1681.469 | 1456.791 | | 10.7802534 |
| 1240 | 26.327 | 1680.917 | 562451.125 | | 4162.13833 |
| 1241 | 50.711 | 1680.552 | 14415.57 | | 106.675218 |
| 1242 | 23.374 | 1680.067 | 13454.194 | | 99.5610356 |
| 1243 | 22.867 | 1680.06 | 1558934.5 | | 11536.1153 |
| 1244 | 22.348 | 1678.716 | 915839.25 | | 6777.21045 |
| 1245 | 20.024 | 1676.707 | 15076.088 | | 111.563051 |
| 1246 | 51.162 | 1675.553 | 15218.188 | | 112.614591 |
| 1247 | 21.999 | 1674.767 | 657963.75 | | 4868.93175 |
| 1248 | 26.48 | 1674.765 | 332114.281 | | 2457.64568 |
| 1249 | 35.911 | 1674.743 | 13065.354 | | 96.6836196 |
| 1250 | 33.635 | 1673.848 | 621973.75 | | 4602.60575 |
| 1251 | 37.217 | 1673.808 | 10630.163 | | 78.6632062 |
| 1252 | 31.406 | 1673.712 | 59847.359 | | 442.870457 |
| 1253 | 60.508 | 1673.41 | 3005.677 | | 22.2420098 |
| 1254 | 26.223 | 1673.293 | 89194.797 | | 660.041498 |
| 1255 | 20.472 | 1672.139 | 5100.346 | | 37.7425604 |
| 1256 | 37.138 | 1669.765 | 1178668.63 | | 8722.14783 |
| 1257 | 21.202 | 1669.559 | 36920.801 | | 273.213927 |
| 1258 | 51.231 | 1669.502 | 17024.83 | | 125.983742 |
| 1259 | 51.002 | 1668.263 | 32127.834 | | 237.745972 |
| 1260 | 22.682 | 1667.961 | 109042.328 | | 806.913227 |
| 1261 | 19.391 | 1667.748 | 14688.896 | | 108.69783 |
| 1262 | 26.233 | 1664.869 | 140821.266 | | 1042.07737 |
| 1263 | 50.726 | 1664.583 | 47740.727 | | 353.28138 |
| 1264 | 19.792 | 1663.743 | 15229.608 | | 112.699099 |
| 1265 | 23.647 | 1662.778 | 25791.463 | | 190.856826 |
| 1266 | 22.348 | 1662.716 | 194397.094 | | 1438.5385 |
| 1267 | 32.006 | 1662.609 | 175913.266 | | 1301.75817 |
| 1268 | 22.899 | 1661.776 | 484126.406 | | 3582.5354 |
| 1269 | 26.062 | 1659.819 | 69192.805 | | 512.026757 |
| 1270 | 26.413 | 1658.822 | 156914.141 | | 1161.16464 |
| 1271 | 22.01 | 1658.774 | 394689.219 | | 2920.70022 |
| 1272 | 25.845 | 1658.703 | 63723.551 | | 471.554277 |
| 1273 | 33.461 | 1657.828 | 80278.719 | | 594.062521 |
| 1274 | 20.167 | 1657.767 | 21783.213 | | 161.195776 |
| 1275 | 26.24 | 1655.771 | 115510.375 | | 854.776775 |
| 1276 | 25.813 | 1655.737 | 9132.748 | | 67.5823352 |
| 1277 | 20.067 | 1655.57 | 79395.461 | | 587.526411 |
| 1278 | 33.143 | 1654.782 | 45294.531 | | 335.179529 |
| 1279 | 26.579 | 1654.285 | 154289.203 | | 1141.7401 |
| 1280 | 39.156 | 1653.766 | 4578.064 | | 33.8776736 |
| 1281 | 51.132 | 1653.527 | 58067.93 | | 429.702682 |
| 1282 | 36.793 | 1653.059 | 534220.188 | | 3953.22939 |
| 1283 | 19.419 | 1652.746 | 15048.502 | | 111.358915 |
| 1284 | 33.485 | 1651.875 | 413598 | | 3060.6252 |
| 1285 | 34.676 | 1651.855 | 16018.068 | | 118.533703 |
| 1286 | 54.087 | 1651.453 | 14638.714 | | 108.326484 |
| 1287 | 19.538 | 1649.803 | 85486.398 | | 632.599345 |
| 1288 | 26.166 | 1649.782 | 13777.515 | | 101.953611 |
| 1289 | 22.145 | 1649.26 | 149952.375 | | 1109.64758 |
| 1290 | 45.247 | 1649.172 | 16909.164 | | 125.127814 |
| 1291 | 26.493 | 1647.797 | 148031.422 | | 1095.43252 |
| 1292 | 27.777 | 1647.787 | 11980.709 | | 88.6572466 |
| 1293 | 19.142 | 1647.559 | 26187.014 | | 193.783904 |
| 1294 | 60.612 | 1647.414 | 8742.313 | | 64.6931162 |
| 1295 | 20.618 | 1645.815 | 32788.332 | | 242.633657 |
| 1296 | 22.892 | 1645.794 | 215293.266 | | 1593.17017 |
| 1297 | 39.588 | 1644.614 | 24574.563 | | 181.851766 |
| 1298 | 40.996 | 1644.614 | 11609.523 | | 85.9104702 |
| 1299 | 41.529 | 1644.612 | 19104.215 | | 141.371191 |
| 1300 | 26.136 | 1643.782 | 40699.555 | | 301.176707 |
| 1301 | 50.713 | 1642.598 | 458311.781 | | 3391.50718 |
| 1302 | 22.368 | 1640.664 | 1787474 | | 13227.3076 |
| 1303 | 26.149 | 1639.423 | 79805.805 | | 590.562957 |
| 1304 | 20.164 | 1638.929 | 446489.313 | | 3304.02092 |
| 1305 | 31.575 | 1637.789 | 17758.281 | | 131.411279 |
| 1306 | 19.439 | 1637.762 | 13106.555 | | 96.988507 |
| 1307 | 33.079 | 1637.729 | 101983 | | 754.6742 |
| 1308 | 35.229 | 1637.691 | 8278.406 | | 61.2602044 |
| 1309 | 56.178 | 1637.442 | 1820.801 | | 13.4739274 |
| 1310 | 22.209 | 1636.865 | 1771329.38 | | 13107.8374 |
| 1311 | 19.979 | 1636.77 | 55767.305 | | 412.678057 |
| 1312 | 26.466 | 1636.56 | 493809.594 | | 3654.191 |
| 1313 | 33.565 | 1635.841 | 15748.322 | | 116.537583 |
| 1314 | 20.32 | 1635.767 | 14488.574 | | 107.215448 |
| 1315 | 26.222 | 1634.903 | 57033.582 | | 422.048507 |
| 1316 | 26.766 | 1634.835 | 31530.076 | | 233.322562 |
| 1317 | 30.976 | 1634.755 | 38005.172 | | 281.238273 |
| 1318 | 26.945 | 1633.589 | 57031.723 | | 422.03475 |
| 1319 | 56.093 | 1633.539 | 4547.565 | | 33.651981 |
| 1320 | 19.445 | 1633.168 | 33451.688 | | 247.542491 |
| 1321 | 33.285 | 1632.811 | 344321.844 | | 2547.98165 |
| 1322 | 37.063 | 1631.805 | 1000832.06 | | 7406.15727 |
| 1323 | 51.14 | 1631.544 | 14658.414 | | 108.472264 |
| 1324 | 19.444 | 1630.801 | 9351.153 | | 69.1985322 |
| 1325 | 20.708 | 1629.062 | 41170.219 | | 304.659621 |
| 1326 | 31.555 | 1628.503 | 27959.01 | | 206.896674 |
| 1327 | 26.276 | 1627.183 | 152365.281 | | 1127.50308 |
| 1328 | 49.992 | 1626.622 | 129430.719 | | 957.787321 |
| 1329 | 51.37 | 1626.608 | 221118.734 | | 1636.27863 |
| 1330 | 23.667 | 1624.787 | 74086.891 | | 548.242993 |
| 1331 | 31.977 | 1624.65 | 151979.656 | | 1124.64945 |
| 1332 | 22.91 | 1623.802 | 813636.25 | | 6020.90825 |
| 1333 | 20.049 | 1623.721 | 48035.508 | | 355.462759 |
| 1334 | 26.115 | 1622.752 | 54070.082 | | 400.118607 |
| 1335 | 26.587 | 1622.569 | 78495.656 | | 580.867854 |
| 1336 | 31.614 | 1622.541 | 48132.473 | | 356.1803 |
| 1337 | 51.449 | 1622.516 | 42449.113 | | 314.123436 |
| 1338 | 19.446 | 1621.752 | 14741.807 | | 109.089372 |
| 1339 | 33.103 | 1621.727 | 169860.656 | | 1256.96885 |
| 1340 | 36.26 | 1621.714 | 10330.106 | | 76.4427844 |
| 1341 | 25.866 | 1620.713 | 85293.617 | | 631.172766 |
| 1342 | 48.018 | 1619.242 | 4286.134 | | 31.7173916 |
| 1343 | 54.868 | 1618.342 | 4299.275 | | 31.814635 |
| 1344 | 26.239 | 1617.782 | 276657.656 | | 2047.26665 |
| 1345 | 55.144 | 1617.691 | 7426.702 | | 54.9575948 |
| 1346 | 33.243 | 1616.145 | 397344.281 | | 2940.34768 |
| 1347 | 60.515 | 1615.419 | 12032.381 | | 89.0396194 |
| 1348 | 22.542 | 1614.932 | 209708.531 | | 1551.84313 |
| 1349 | 19.439 | 1614.786 | 43254.602 | | 320.084055 |
| 1350 | 36.176 | 1614.752 | 366360.5 | | 2711.0677 |
| 1351 | 22.89 | 1613.878 | 29076.914 | | 215.169164 |
| 1352 | 31.579 | 1612.537 | 33888.555 | | 250.775307 |
| 1353 | 50.626 | 1610.636 | 57666.223 | | 426.73005 |
| 1354 | 51.796 | 1610.61 | 5026.365 | | 37.195101 |
| 1355 | 49.488 | 1610.521 | 27563.52 | | 203.970048 |
| 1356 | 26.476 | 1609.682 | 249802.609 | | 1848.53931 |
| 1357 | 19.82 | 1607.438 | 22530.867 | | 166.728416 |
| 1358 | 40.415 | 1606.62 | 6549.202 | | 48.4640948 |
| 1359 | 51.431 | 1606.541 | 53775.559 | | 397.939137 |
| 1360 | 60.308 | 1606.455 | 20217.408 | | 149.608819 |
| 1361 | 45.153 | 1606.051 | 104704.188 | | 774.810991 |
| 1362 | 33.093 | 1605.74 | 107556.352 | | 795.917005 |
| 1363 | 52.135 | 1604.625 | 27542.211 | | 203.812361 |
| 1364 | 51.752 | 1604.623 | 19708.334 | | 145.841672 |
| 1365 | 50.377 | 1604.425 | 498449.438 | | 3688.52584 |
| 1366 | 25.879 | 1602.593 | 80256.023 | | 593.89457 |
| 1367 | 20.142 | 1602.029 | 283347.281 | | 2096.76988 |
| 1368 | 26.208 | 1601.483 | 174360.688 | | 1290.26909 |
| 1369 | 21.843 | 1600.709 | 71368.32 | | 528.125568 |
| 1370 | 33.841 | 1599.775 | 1946478.5 | | 14403.9409 |
| 1371 | 26.365 | 1595.636 | 99707.125 | | 737.832725 |
| 1372 | 55.052 | 1595.379 | 13544.353 | | 100.228212 |
| 1373 | 33.255 | 1594.838 | 133055.125 | | 984.607925 |
| 1374 | 19.115 | 1594.616 | 9341.319 | | 69.1257606 |
| 1375 | 19.447 | 1592.784 | 123089.523 | | 910.86247 |
| 1376 | 52.334 | 1590.926 | 19367.498 | | 143.319485 |
| 1377 | 32.656 | 1590.783 | 42339.105 | | 313.309377 |
| 1378 | 27.674 | 1590.767 | 29748.822 | | 220.141283 |
| 1379 | 31.605 | 1590.551 | 240303.016 | | 1778.24232 |
| 1380 | 51.487 | 1590.549 | 28733.951 | | 212.631237 |
| 1381 | 60.485 | 1590.45 | 6380.994 | | 47.2193556 |
| 1382 | 20.136 | 1589.801 | 129593.336 | | 958.990686 |
| 1383 | 26.161 | 1589.765 | 28449.627 | | 210.52724 |
| 1384 | 26.465 | 1588.799 | 144476.25 | | 1069.12425 |
| 1385 | 50.453 | 1588.666 | 91979.852 | | 680.650905 |
| 1386 | 19.006 | 1588.024 | 23308.154 | | 172.48034 |
| 1387 | 32.774 | 1587.746 | 61703.977 | | 456.60943 |
| 1388 | 25.703 | 1587.197 | 49110.512 | | 363.417789 |
| 1389 | 19.518 | 1585.876 | 20618.105 | | 152.573977 |
| 1390 | 51.336 | 1584.592 | 147183.422 | | 1089.15732 |
| 1391 | 31.605 | 1584.465 | 112075.383 | | 829.357834 |
| 1392 | 26.004 | 1584.374 | 152446.016 | | 1128.10052 |
| 1393 | 33.983 | 1583.763 | 1606523.13 | | 11888.2711 |
| 1394 | 19.229 | 1583.691 | 41368.191 | | 306.124613 |
| 1395 | 60.592 | 1583.443 | 10818.154 | | 80.0543396 |
| 1396 | 22.462 | 1583.401 | 51175.965 | | 378.702141 |
| 1397 | 23.455 | 1580.979 | 750475.688 | | 5553.52009 |
| 1398 | 22.879 | 1580.944 | 82073.273 | | 607.34222 |
| 1399 | 26.267 | 1579.821 | 460781.469 | 2618.98951 | 3409.78287 |
| 1400 | 20.062 | 1579.781 | 1163685.25 | | 8611.27085 |
| 1401 | 55.029 | 1579.406 | 35646.781 | | 263.786179 |
| 1402 | 19.223 | 1578.194 | 34980.523 | | 258.85587 |
| 1403 | 34.082 | 1577.542 | 1220426.38 | | 9031.15518 |
| 1404 | 19.43 | 1577.048 | 211572.891 | | 1565.63939 |
| 1405 | 52.329 | 1575.122 | 15242.363 | | 112.793486 |
| 1406 | 31.579 | 1574.581 | 56584.949 | | 418.728623 |
| 1407 | 26.771 | 1573.768 | 11005.183 | | 81.4383542 |
| 1408 | 26.34 | 1573.766 | 88419.383 | | 654.303434 |
| 1409 | 25.642 | 1571.653 | 18782.912 | | 138.993549 |
| 1410 | 32.726 | 1570.316 | 86676.258 | | 641.404309 |
| 1411 | 26.215 | 1569.753 | 18192.246 | | 134.62262 |
| 1412 | 52.301 | 1569.494 | 49528.332 | | 366.509657 |
| 1413 | 25.953 | 1568.74 | 99835.68 | | 738.784032 |
| 1414 | 51.381 | 1568.572 | 103803.664 | | 768.147114 |
| 1415 | 60.515 | 1568.367 | 17354.266 | | 128.421568 |
| 1416 | 33.2 | 1567.797 | 434310.063 | | 3213.89447 |
| 1417 | 20.165 | 1567.795 | 372919.156 | | 2759.60175 |
| 1418 | 35.532 | 1567.755 | 7017.221 | | 51.9274354 |
| 1419 | 34.952 | 1567.754 | 19015.121 | | 140.711895 |
| 1420 | 22.644 | 1567.696 | 14336.802 | | 106.092335 |
| 1421 | 19.412 | 1567.693 | 15762.474 | | 116.642308 |
| 1422 | 25.669 | 1565.649 | 107737.258 | | 797.255709 |
| 1423 | 31.653 | 1563.506 | 256880.891 | | 1900.91859 |
| 1424 | 55.037 | 1563.437 | 31753.439 | | 234.975449 |
| 1425 | 21.99 | 1562.683 | 442783.563 | | 3276.59837 |
| 1426 | 31.395 | 1562.551 | 49908.277 | | 369.32125 |
| 1427 | 45.126 | 1562.09 | 120262.977 | | 889.94603 |
| 1428 | 33.892 | 1561.784 | 4378570.5 | | 32401.4217 |
| 1429 | 26.083 | 1560.761 | 172583.031 | | 1277.11443 |
| 1430 | 54.978 | 1557.43 | 57674.145 | | 426.788673 |
| 1431 | 47.314 | 1557.371 | 2242.944 | | 16.5977856 |
| 1432 | 25.21 | 1556.715 | 21400.275 | | 158.362035 |
| 1433 | 19.157 | 1556.658 | 40701.605 | | 301.191877 |
| 1434 | 25.762 | 1555.668 | 10494.886 | | 77.6621564 |
| 1435 | 52.317 | 1553.479 | 107733.453 | | 797.227552 |
| 1436 | 31.602 | 1552.596 | 164191 | | 1215.0134 |
| 1437 | 60.548 | 1552.244 | 18100.105 | | 133.940777 |
| 1438 | 32.949 | 1552.029 | 108975.336 | | 806.417486 |
| 1439 | 51.293 | 1551.536 | 9862.231 | | 72.9805094 |
| 1440 | 20.335 | 1551.002 | 41674.418 | | 308.390693 |
| 1441 | 26.24 | 1550.756 | 123754.648 | | 915.784395 |
| 1442 | 25.661 | 1549.661 | 181339.203 | | 1341.9101 |
| 1443 | 31.599 | 1547.554 | 126639.313 | | 937.130916 |
| 1444 | 52.2 | 1547.002 | 3902.257 | | 28.8767018 |
| 1445 | 26.148 | 1546.781 | 397945.156 | | 2944.79415 |
| 1446 | 51.365 | 1546.585 | 141115.656 | | 1044.25585 |
| 1447 | 18.965 | 1546.009 | 8138.867 | | 60.2276158 |
| 1448 | 33.874 | 1545.796 | 2482156.75 | | 18367.96 |
| 1449 | 22.718 | 1545.666 | 96470.289 | | 713.880139 |
| 1450 | 25.71 | 1543.679 | 57332.781 | | 424.262579 |
| 1451 | 55.025 | 1541.204 | 81368.375 | | 602.125975 |
| 1452 | 33.187 | 1539.813 | 342826.781 | | 2536.91818 |
| 1453 | 22.859 | 1539.745 | 17030.666 | | 126.026928 |
| 1454 | 26.143 | 1538.782 | 47694.641 | | 352.940343 |
| 1455 | 52.335 | 1537.494 | 74524.922 | | 551.484423 |
| 1456 | 54.213 | 1537.361 | 3714.262 | | 27.4855388 |
| 1457 | 20.394 | 1536.787 | 47255.895 | | 349.693623 |
| 1458 | 60.536 | 1536.53 | 5179.255 | | 38.326487 |
| 1459 | 26.437 | 1535.394 | 206936.766 | | 1531.33207 |
| 1460 | 26.032 | 1534.45 | 21739.525 | | 160.872485 |
| 1461 | 25.657 | 1533.617 | 116876.422 | | 864.885523 |
| 1462 | 31.429 | 1533.537 | 35516.992 | | 262.825741 |
| 1463 | 32.719 | 1533.021 | 52503.387 | | 388.525064 |
| 1464 | 52.327 | 1531.486 | 189285.625 | | 1400.71363 |
| 1465 | 58.795 | 1531.259 | 3579.956 | | 26.4916744 |
| 1466 | 25.934 | 1530.757 | 53750.309 | | 397.752287 |
| 1467 | 33.111 | 1529.795 | 354812.281 | | 2625.61088 |
| 1468 | 51.31 | 1529.754 | 68137.281 | | 504.215879 |
| 1469 | 22.669 | 1529.696 | 97531.555 | | 721.733507 |
| 1470 | 25.708 | 1527.677 | 557475 | | 4125.315 |
| 1471 | 21.377 | 1525.647 | 95958.109 | | 710.090007 |
| 1472 | 31.604 | 1525.585 | 224192.984 | | 1659.02808 |
| 1473 | 21.109 | 1524.714 | 50138.973 | | 371.0284 |
| 1474 | 20.247 | 1524.422 | 26032.813 | | 192.642816 |
| 1475 | 21.84 | 1523.845 | 1315288.25 | | 9733.13305 |
| 1476 | 33.882 | 1523.824 | 2137835.75 | | 15819.9846 |
| 1477 | 22.403 | 1522.807 | 56880.883 | | 420.918534 |
| 1478 | 26.05 | 1522.795 | 225871.531 | | 1671.44933 |
| 1479 | 20.691 | 1522.736 | 65239.566 | | 482.772788 |
| 1480 | 32.659 | 1522.66 | 48612.965 | | 359.735941 |
| 1481 | 52.275 | 1521.537 | 11822.047 | | 87.4831478 |
| 1482 | 55.043 | 1519.4 | 112879.547 | | 835.308648 |
| 1483 | 19.173 | 1518.7 | 310531.188 | | 2297.93079 |
| 1484 | 45.108 | 1518.042 | 159780.984 | | 1182.37928 |
| 1485 | 42.505 | 1517.544 | 9947.583 | | 73.6121142 |
| 1486 | 32.712 | 1517.013 | 81436.086 | | 602.627036 |
| 1487 | 52.328 | 1515.49 | 260667 | | 1928.9358 |
| 1488 | 51.064 | 1515.057 | 40951.52 | | 303.041248 |
| 1489 | 25.304 | 1514.678 | 105488.594 | | 780.615596 |
| 1490 | 31.295 | 1514.467 | 213922.516 | | 1583.02662 |
| 1491 | 58.997 | 1514.391 | 2223.704 | | 16.4554096 |
| 1492 | 33.008 | 1513.778 | 31166.486 | | 230.631996 |
| 1493 | 51.393 | 1513.492 | 6028.764 | | 44.6128536 |
| 1494 | 25.684 | 1511.67 | 533613.25 | | 3948.73805 |
| 1495 | 59.581 | 1511.401 | 7321.546 | | 54.1794404 |
| 1496 | 20.163 | 1510.747 | 120274.336 | | 890.030086 |
| 1497 | 23.298 | 1510.736 | 31251.807 | | 231.263372 |
| 1498 | 52.276 | 1509.5 | 7264.465 | | 53.757041 |
| 1499 | 21.434 | 1509.107 | 22580.268 | | 167.093983 |
| 1500 | 26.035 | 1508.878 | 416968.781 | 2402.66451 | 3085.56898 |
| 1501 | 21.099 | 1508.716 | 26668.498 | | 197.346885 |
| 1502 | 22.714 | 1507.972 | 296770.906 | | 2196.1047 |
| 1503 | 33.166 | 1507.821 | 377186.125 | | 2791.17733 |
| 1504 | 19.343 | 1507.692 | 21455.111 | | 158.767821 |
| 1505 | 51.294 | 1507.543 | 59747.996 | | 442.13517 |
| 1506 | 25.715 | 1505.656 | 93126.773 | | 689.13812 |
| 1507 | 21.393 | 1504.976 | 224635.625 | | 1662.30363 |
| 1508 | 31.026 | 1504.421 | 61428.184 | | 454.568562 |
| 1509 | 55.107 | 1503.301 | 55199.215 | | 408.474191 |
| 1510 | 22.308 | 1500.84 | 38119.215 | | 282.082191 |
| 1511 | 19.418 | 1500.692 | 21493.119 | | 159.049081 |
| 1512 | 21.883 | 1500.677 | 26562.451 | | 196.562137 |
| 1513 | 32.646 | 1500.676 | 267310.469 | | 1978.09747 |
| 1514 | 52.314 | 1499.535 | 113653.813 | | 841.038216 |
| 1515 | 51.11 | 1499.471 | 22515.533 | | 166.614944 |
| 1516 | 31.725 | 1499.319 | 25246.293 | | 186.822568 |
| 1517 | 31.342 | 1498.522 | 205765.641 | | 1522.66574 |
| 1518 | 56.804 | 1497.389 | 8472.941 | | 62.6997634 |
| 1519 | 31.431 | 1495.577 | 37132.027 | | 274.777 |
| 1520 | 25.701 | 1495.51 | 180634.594 | | 1336.696 |
| 1521 | 59.603 | 1495.327 | 12399.307 | | 91.7548718 |
| 1522 | 45.048 | 1494.97 | 42989.801 | | 318.124527 |
| 1523 | 26.474 | 1494.756 | 60827.902 | | 450.126475 |
| 1524 | 56.739 | 1494.4 | 15389.742 | | 113.884091 |
| 1525 | 50.703 | 1494.051 | 49096.602 | | 363.314855 |
| 1526 | 52.323 | 1493.535 | 513625.938 | | 3800.83194 |
| 1527 | 58.794 | 1493.276 | 3180.229 | | 23.5336946 |
| 1528 | 33.034 | 1491.809 | 39573.719 | | 292.845521 |
| 1529 | 22.69 | 1491.715 | 62520.961 | | 462.655111 |
| 1530 | 51.31 | 1490.596 | 64390.566 | | 476.490188 |
| 1531 | 25.699 | 1489.703 | 1324499.13 | | 9801.29353 |
| 1532 | 27.749 | 1489.695 | 35656.855 | | 263.860727 |
| 1533 | 21.937 | 1488.703 | 58306.789 | | 431.470239 |
| 1534 | 26.674 | 1488.673 | 12532.065 | | 92.737281 |
| 1535 | 26.864 | 1488.669 | 9989.638 | | 73.9233212 |
| 1536 | 31.025 | 1488.435 | 115011.352 | | 851.084005 |
| 1537 | 20.064 | 1487.66 | 60555.18 | | 448.108332 |
| 1538 | 57.5 | 1487.261 | 22979.725 | | 170.049965 |
| 1539 | 21.102 | 1486.746 | 86114.773 | | 637.24932 |
| 1540 | 20.302 | 1485.814 | 35362.301 | | 261.681027 |
| 1541 | 19.391 | 1485.744 | 35368.121 | | 261.724095 |
| 1542 | 22.765 | 1485.739 | 325309.406 | | 2407.2896 |
| 1543 | 21.7 | 1485.577 | 179972.781 | | 1331.79858 |
| 1544 | 32.638 | 1484.686 | 284208.563 | | 2103.14337 |
| 1545 | 20.646 | 1483.769 | 99830.844 | | 738.748246 |
| 1546 | 31.547 | 1483.497 | 66147.68 | | 489.492832 |
| 1547 | 21.97 | 1483.484 | 101875.852 | | 753.881305 |
| 1548 | 25.512 | 1482.712 | 47738.512 | | 353.264989 |
| 1549 | 59.952 | 1482.332 | 3723.846 | | 27.5564604 |
| 1550 | 54.947 | 1481.514 | 27755.322 | | 205.389383 |
| 1551 | 51.342 | 1480.523 | 8275.503 | | 61.2387222 |
| 1552 | 59.684 | 1480.179 | 10350.676 | | 76.5950024 |
| 1553 | 19.868 | 1479.653 | 9464.678 | | 70.0386172 |
| 1554 | 51.641 | 1479.497 | 11117.603 | | 82.2702622 |
| 1555 | 32.695 | 1478.703 | 170849.156 | | 1264.28375 |
| 1556 | 56.784 | 1478.422 | 25985.34 | | 192.291516 |
| 1557 | 19.792 | 1477.648 | 12052.142 | | 89.1858508 |
| 1558 | 52.023 | 1477.537 | 506844.188 | | 3750.64699 |
| 1559 | 26.012 | 1476.751 | 22272.973 | | 164.82 |
| 1560 | 25.289 | 1476.723 | 292965.156 | | 2167.94215 |
| 1561 | 31.286 | 1476.502 | 618812.75 | | 4579.21435 |
| 1562 | 50.115 | 1475.578 | 34773.316 | | 257.322538 |
| 1563 | 42.959 | 1475.564 | 14523.597 | | 107.474618 |
| 1564 | 42.656 | 1475.558 | 14509.699 | | 107.371773 |
| 1565 | 18.991 | 1474.936 | 168177.938 | | 1244.51674 |
| 1566 | 59.531 | 1474.323 | 12192.879 | | 90.2273046 |
| 1567 | 45.092 | 1474.032 | 172501.25 | | 1276.50925 |
| 1568 | 25.694 | 1473.735 | 732376.563 | | 5419.58657 |
| 1569 | 22.004 | 1473.732 | 141438.281 | | 1046.64328 |
| 1570 | 57.184 | 1472.278 | 4585.247 | | 33.9308278 |
| 1571 | 58.089 | 1471.285 | 3652.961 | | 27.0319114 |
| 1572 | 21.032 | 1470.737 | 39272.316 | | 290.615138 |
| 1573 | 22.699 | 1469.663 | 479302.844 | | 3546.84105 |
| 1574 | 51.293 | 1469.563 | 30035.428 | | 222.262167 |
| 1575 | 23.445 | 1467.89 | 471461.313 | | 3488.81372 |
| 1576 | 22.953 | 1467.864 | 57678.805 | | 426.823157 |
| 1577 | 22.184 | 1467.716 | 12644.103 | | 93.5663622 |
| 1578 | 21.462 | 1466.901 | 629920.375 | | 4661.41078 |
| 1579 | 25.577 | 1466.726 | 42258.711 | | 312.714461 |
| 1580 | 31.043 | 1466.436 | 690857.25 | | 5112.34365 |
| 1581 | 57.749 | 1466.294 | 25648.756 | | 189.800794 |
| 1582 | 26.832 | 1464.747 | 59844.988 | | 442.852911 |
| 1583 | 52.112 | 1464.549 | 33567.551 | | 248.399877 |
| 1584 | 19.391 | 1463.779 | 92841.156 | | 687.024554 |
| 1585 | 21.836 | 1463.622 | 277416.719 | | 2052.88372 |
| 1586 | 18.809 | 1462.708 | 7145.363 | | 52.8756862 |
| 1587 | 33.265 | 1462.706 | 1376367.75 | | 10185.1214 |
| 1588 | 26.317 | 1461.692 | 7774.391 | | 57.5304934 |
| 1589 | 52.389 | 1461.566 | 83025.867 | | 614.391416 |
| 1590 | 51.105 | 1461.536 | 144334.891 | | 1068.07819 |
| 1591 | 31.735 | 1461.262 | 232331.219 | | 1719.25102 |
| 1592 | 21.435 | 1460.957 | 169996.313 | | 1257.97272 |
| 1593 | 25.385 | 1460.724 | 306236.313 | | 2266.14872 |
| 1594 | 31.326 | 1460.56 | 238888.328 | | 1767.77363 |
| 1595 | 59.746 | 1458.356 | 37428.387 | | 276.970064 |
| 1596 | 25.61 | 1457.725 | 35071.652 | | 259.530225 |
| 1597 | 20.028 | 1455.677 | 29577.705 | | 218.875017 |
| 1598 | 52.304 | 1455.576 | 331660.813 | | 2454.29002 |
| 1599 | 51.028 | 1455.55 | 35265.898 | | 260.967645 |
| 1600 | 60.356 | 1453.339 | 4032.295 | | 29.838983 |
| 1601 | 45.098 | 1453.108 | 32228.975 | | 238.494415 |
| 1602 | 22.717 | 1452.713 | 45172.172 | | 334.274073 |
| 1603 | 20.322 | 1452.661 | 11800.213 | | 87.3215762 |
| 1604 | 25.906 | 1451.759 | 2339633.5 | 20870.565 | 17313.2879 |
| 1605 | 22.074 | 1451.69 | 248246.688 | | 1837.02549 |
| 1606 | 59.326 | 1451.369 | 4813.728 | | 35.6215872 |
| 1607 | 59.701 | 1451.353 | 1536.576 | | 11.3706624 |
| 1608 | 58.268 | 1451.321 | 3431.627 | | 25.3940398 |
| 1609 | 58.798 | 1451.281 | 5105.037 | | 37.7772738 |
| 1610 | 45.048 | 1450.942 | 91047.344 | | 673.750346 |
| 1611 | 23.167 | 1450.662 | 13077.415 | | 96.772871 |
| 1612 | 31.043 | 1450.47 | 449713.969 | | 3327.88337 |
| 1613 | 21.432 | 1449.717 | 434321.813 | | 3213.98142 |
| 1614 | 22.084 | 1448.686 | 19411.707 | | 143.646632 |
| 1615 | 19.402 | 1447.709 | 167506.141 | | 1239.54544 |
| 1616 | 21.69 | 1447.618 | 597990.688 | | 4425.13109 |
| 1617 | 42.128 | 1447.568 | 8425.077 | | 62.3455698 |
| 1618 | 59.775 | 1447.348 | 5009.105 | | 37.067377 |
| 1619 | 32.688 | 1446.725 | 572850 | | 4239.09 |
| 1620 | 54.385 | 1446.459 | 14195.906 | | 105.049704 |
| 1621 | 21.355 | 1445.637 | 101465.133 | | 750.841984 |
| 1622 | 31.582 | 1445.631 | 231467.766 | | 1712.86147 |
| 1623 | 51.134 | 1445.524 | 69437.68 | | 513.838832 |
| 1624 | 52.726 | 1445.505 | 4868.882 | | 36.0297268 |
| 1625 | 25.539 | 1444.755 | 80478.234 | | 595.538932 |
| 1626 | 20.78 | 1444.136 | 11996.748 | | 88.7759352 |
| 1627 | 20.099 | 1443.858 | 48822.273 | | 361.28482 |
| 1628 | 32.312 | 1443.65 | 21127.52 | | 156.343648 |
| 1629 | 41.941 | 1443.593 | 224228.266 | | 1659.28917 |
| 1630 | 47.591 | 1442.561 | 5376.899 | | 39.7890526 |
| 1631 | 59.774 | 1442.484 | 29060.875 | | 215.050475 |
| 1632 | 52.084 | 1442.129 | 9874.53 | | 73.071522 |
| 1633 | 56.749 | 1440.464 | 17626.166 | | 130.433628 |
| 1634 | 53.231 | 1440.327 | 7973.153 | | 59.0013322 |
| 1635 | 51.285 | 1439.505 | 721402.5 | | 5338.3785 |
| 1636 | 30.909 | 1439.44 | 47137.531 | | 348.817729 |
| 1637 | 25.302 | 1438.762 | 476602.969 | | 3526.86197 |
| 1638 | 26.098 | 1438.714 | 131795.766 | | 975.288668 |
| 1639 | 31.282 | 1438.543 | 532467.688 | | 3940.26089 |
| 1640 | 59.661 | 1436.368 | 26654.971 | | 197.246785 |
| 1641 | 25.623 | 1435.749 | 403066.875 | 5145.30946 | 2982.69488 |
| 1642 | 57.527 | 1434.335 | 61294.555 | | 453.579707 |
| 1643 | 19.291 | 1430.597 | 19614.697 | | 145.148758 |
| 1644 | 45.07 | 1430.006 | 183551.078 | | 1358.27798 |
| 1645 | 31.217 | 1428.461 | 2081349.38 | | 15401.9854 |
| 1646 | 41.061 | 1427.616 | 25458.787 | | 188.395024 |
| 1647 | 21.887 | 1425.708 | 515758.594 | | 3816.6136 |
| 1648 | 32.632 | 1424.748 | 292768.469 | | 2166.48667 |
| 1649 | 20.188 | 1423.596 | 59391.871 | | 439.499845 |
| 1650 | 51.159 | 1423.54 | 678686.125 | | 5022.27733 |
| 1651 | 19.388 | 1423.463 | 14447.822 | | 106.913883 |
| 1652 | 60.406 | 1423.454 | 6015.27 | | 44.512998 |
| 1653 | 21.462 | 1423.091 | 689747.75 | | 5104.13335 |
| 1654 | 31.762 | 1423.015 | 158091.891 | | 1169.87999 |
| 1655 | 25.58 | 1422.755 | 154930.813 | | 1146.48802 |
| 1656 | 59.962 | 1420.822 | 60185.059 | | 445.369437 |
| 1657 | 47.513 | 1420.228 | 39887.438 | | 295.167041 |
| 1658 | 25.381 | 1419.602 | 160612.406 | | 1188.5318 |
| 1659 | 22.881 | 1419.568 | 164075.094 | | 1214.1557 |
| 1660 | 59.173 | 1418.341 | 8607.118 | | 63.6926732 |
| 1661 | 25.701 | 1416.669 | 250310.063 | | 1852.29447 |
| 1662 | 19.933 | 1415.938 | 15313.807 | | 113.322172 |
| 1663 | 59.853 | 1415.354 | 2729.604 | | 20.1990696 |
| 1664 | 60.002 | 1414.327 | 2912.038 | | 21.5490812 |
| 1665 | 25.344 | 1413.599 | 40637.359 | | 300.716457 |
| 1666 | 23.791 | 1413.597 | 50547.738 | | 374.053261 |
| 1667 | 45.052 | 1412.986 | 13919.365 | | 103.003301 |
| 1668 | 25.335 | 1412.84 | 14382.029 | | 106.427015 |
| 1669 | 32.132 | 1412.554 | 54933.371 | | 406.506945 |
| 1670 | 31.045 | 1412.512 | 527367.25 | | 3902.51765 |
| 1671 | 57.556 | 1412.347 | 25780.541 | | 190.776003 |
| 1672 | 32.409 | 1411.64 | 16508.381 | | 122.162019 |
| 1673 | 59.681 | 1410.329 | 5465.604 | | 40.4454696 |
| 1674 | 31.527 | 1407.728 | 41320.148 | | 305.769095 |
| 1675 | 21.351 | 1407.672 | 178818.625 | | 1323.25783 |
| 1676 | 51.17 | 1407.562 | 118187.75 | | 874.58935 |
| 1677 | 52.677 | 1407.503 | 40964.031 | | 303.133829 |
| 1678 | 45.069 | 1406.92 | 71280.117 | | 527.472866 |
| 1679 | 59.199 | 1406.339 | 3843.809 | | 28.4441866 |
| 1680 | 60.013 | 1406.165 | 13561.318 | | 100.353753 |
| 1681 | 22.464 | 1405.749 | 23630.906 | | 174.868704 |
| 1682 | 20.155 | 1405.702 | 79460.164 | | 588.005214 |
| 1683 | 32.604 | 1405.687 | 482839.156 | | 3573.00975 |
| 1684 | 41.281 | 1405.646 | 101633.367 | | 752.086916 |
| 1685 | 59.693 | 1405.026 | 18004.275 | | 133.231635 |
| 1686 | 25.352 | 1403.563 | 92161.961 | | 681.998511 |
| 1687 | 53.201 | 1402.099 | 23272.326 | | 172.215212 |
| 1688 | 51.072 | 1401.522 | 388249.438 | | 2873.04584 |
| 1689 | 31.654 | 1401.509 | 7511.224 | | 55.5830576 |
| 1690 | 30.98 | 1401.473 | 171155.313 | | 1266.54932 |
| 1691 | 25.705 | 1400.678 | 71240.352 | | 527.178605 |
| 1692 | 60.454 | 1399.435 | 15121.45 | | 111.89873 |
| 1693 | 19.068 | 1397.692 | 3839.199 | | 28.4100726 |
| 1694 | 25.389 | 1397.57 | 528235.875 | | 3908.94548 |
| 1695 | 32.288 | 1396.606 | 10441.645 | | 77.268173 |
| 1696 | 31.138 | 1396.434 | 127553.898 | | 943.898845 |
| 1697 | 57.725 | 1396.36 | 55281.977 | | 409.08663 |
| 1698 | 21.421 | 1392.695 | 709464.313 | | 5250.03592 |
| 1699 | 59.374 | 1391.448 | 4601.684 | | 34.0524616 |
| 1700 | 59.651 | 1391.428 | 2721.883 | | 20.1419342 |
| 1701 | 45.062 | 1390.986 | 37551.5 | | 277.8811 |
| 1702 | 31.091 | 1390.532 | 1131354.75 | | 8372.02515 |
| 1703 | 32.449 | 1389.93 | 406237.781 | | 3006.15958 |
| 1704 | 43.567 | 1388.482 | 94132.688 | | 696.581891 |
| 1705 | 51.546 | 1386.784 | 3704.049 | | 27.4099626 |
| 1706 | 25.04 | 1386.521 | 93427.875 | | 691.366275 |
| 1707 | 59.273 | 1386.36 | 24964.27 | | 184.735598 |
| 1708 | 45.063 | 1385.978 | 210238.953 | | 1555.76825 |
| 1709 | 51.138 | 1385.566 | 41882.465 | | 309.930241 |
| 1710 | 30.901 | 1385.488 | 20099.48 | | 148.736152 |
| 1711 | 59.7 | 1382.403 | 30916.771 | | 228.784105 |
| 1712 | 47.077 | 1382.273 | 64972.203 | | 480.794302 |
| 1713 | 25.349 | 1381.698 | 426013.25 | | 3152.49805 |
| 1714 | 21.186 | 1380.693 | 20415.264 | | 151.072954 |
| 1715 | 25.109 | 1380.547 | 10967.781 | | 81.1615794 |
| 1716 | 53.108 | 1380.342 | 13301.506 | | 98.4311444 |
| 1717 | 25.679 | 1378.704 | 342898.781 | 2415.39444 | 2537.45098 |
| 1718 | 49.632 | 1378.394 | 5379.274 | | 39.8066276 |
| 1719 | 18.97 | 1377.85 | 30348.645 | | 224.579973 |
| 1720 | 25.346 | 1375.624 | 46687.473 | | 345.4873 |
| 1721 | 32.125 | 1375.532 | 67528.18 | | 499.708532 |
| 1722 | 37.369 | 1375.445 | 8201.093 | | 60.6880882 |
| 1723 | 34.801 | 1375.438 | 17825.877 | | 131.91149 |
| 1724 | 55.31 | 1374.42 | 31385.559 | | 232.253137 |
| 1725 | 56.785 | 1374.415 | 9927.623 | | 73.4644102 |
| 1726 | 32.405 | 1373.689 | 38635.266 | | 285.900968 |
| 1727 | 60.227 | 1373.36 | 6000.033 | | 44.4002442 |
| 1728 | 45.05 | 1368.949 | 34093.426 | | 252.291352 |
| 1729 | 32.464 | 1367.728 | 132403.016 | | 979.782318 |
| 1730 | 55.22 | 1367.496 | 1103.651 | | 8.1670174 |
| 1731 | 60.375 | 1366.465 | 12431.157 | | 91.9905618 |
| 1732 | 25.359 | 1365.647 | 47731.473 | | 353.2129 |
| 1733 | 45.052 | 1365.037 | 104198.375 | | 771.067975 |
| 1734 | 19.189 | 1364.748 | 11513.005 | | 85.196237 |
| 1735 | 25.813 | 1364.605 | 75452.961 | | 558.351911 |
| 1736 | 25.024 | 1364.429 | 298666.156 | | 2210.12955 |
| 1737 | 51.066 | 1363.59 | 4498.999 | | 33.2925926 |
| 1738 | 30.979 | 1363.458 | 95695.992 | | 708.150341 |
| 1739 | 45.02 | 1362.89 | 110947.891 | | 821.014393 |
| 1740 | 32.068 | 1359.662 | 52750.859 | | 390.356357 |
| 1741 | 25.534 | 1359.656 | 1906445.75 | | 14107.6986 |
| 1742 | 31.049 | 1358.466 | 151038.281 | | 1117.68328 |
| 1743 | 49.608 | 1356.381 | 45604.996 | | 337.47697 |
| 1744 | 59.708 | 1356.319 | 29863.813 | | 220.992216 |
| 1745 | 60.659 | 1355.61 | 1210.575 | | 8.958255 |
| 1746 | 55.572 | 1354.558 | 1049.276 | | 7.7646424 |
| 1747 | 58.894 | 1354.391 | 2738.662 | | 20.2660988 |
| 1748 | 23.999 | 1353.72 | 101441.016 | | 750.663518 |
| 1749 | 23.416 | 1352.847 | 43856.227 | | 324.53608 |
| 1750 | 30.605 | 1352.398 | 90038.555 | | 666.285307 |
| 1751 | 32.39 | 1351.686 | 32230.543 | | 238.506018 |
| 1752 | 34.799 | 1351.387 | 8059.731 | | 59.6420094 |
| 1753 | 25.989 | 1350.605 | 40075.652 | | 296.559825 |
| 1754 | 59.605 | 1350.549 | 6613.854 | | 48.9425196 |
| 1755 | 59.372 | 1349.363 | 5556.154 | | 41.1155396 |
| 1756 | 25.073 | 1348.535 | 290868.375 | | 2152.42598 |
| 1757 | 18.811 | 1347.718 | 12626.824 | | 93.4384976 |
| 1758 | 25.458 | 1343.664 | 766104.375 | | 5669.17238 |
| 1759 | 26.465 | 1342.628 | 17343.17 | | 128.339458 |
| 1760 | 53.156 | 1342.392 | 5754.031 | | 42.5798294 |
| 1761 | 45.041 | 1341.946 | 191931.266 | | 1420.29137 |
| 1762 | 29.936 | 1339.384 | 45313.438 | | 335.319441 |
| 1763 | 22.748 | 1338.66 | 77636.578 | | 574.510677 |
| 1764 | 60.353 | 1338.395 | 2928.564 | | 21.6713736 |
| 1765 | 30.621 | 1336.417 | 37646.219 | | 278.582021 |
| 1766 | 55.091 | 1334.981 | 17385.801 | | 128.654927 |
| 1767 | 59.445 | 1334.35 | 36656.449 | | 271.257723 |
| 1768 | 19.066 | 1332.751 | 11472.639 | | 84.8975286 |
| 1769 | 25.099 | 1332.551 | 113287.906 | | 838.330504 |
| 1770 | 59.549 | 1330.309 | 2890.745 | | 21.391513 |
| 1771 | 59.275 | 1330.277 | 9237.706 | | 68.3590244 |
| 1772 | 25.154 | 1326.551 | 949441.625 | | 7025.86803 |
| 1773 | 45.045 | 1324.926 | 42727.93 | | 316.186682 |
| 1774 | 31.961 | 1322.603 | 28099.598 | | 207.937025 |
| 1775 | 25.586 | 1321.685 | 2478338.5 | | 18339.7049 |
| 1776 | 30.808 | 1321.321 | 58397.359 | | 432.140457 |
| 1777 | 45.004 | 1321.021 | 66336.734 | | 490.891832 |
| 1778 | 20.636 | 1320.662 | 25817.596 | | 191.05021 |
| 1779 | 32.429 | 1319.441 | 11738.492 | | 86.8648408 |
| 1780 | 31.4 | 1319.42 | 6886.302 | | 50.9586348 |
| 1781 | 52.954 | 1319.408 | 3111.002 | | 23.0214148 |
| 1782 | 50.24 | 1319.394 | 4092.932 | | 30.2876968 |
| 1783 | 48.515 | 1319.389 | 12137.362 | | 89.8164788 |
| 1784 | 34.136 | 1319.387 | 43983.848 | | 325.480475 |
| 1785 | 35.227 | 1319.362 | 4880.322 | | 36.1143828 |
| 1786 | 45 | 1318.861 | 87233.422 | | 645.527323 |
| 1787 | 31.907 | 1318.611 | 13398.739 | | 99.1506686 |
| 1788 | 41.768 | 1318.589 | 232563.891 | | 1720.97279 |
| 1789 | 39.529 | 1318.407 | 9566.113 | | 70.7892362 |
| 1790 | 46.954 | 1318.405 | 13212.546 | | 97.7728404 |
| 1791 | 53.154 | 1318.387 | 4069.485 | | 30.114189 |
| 1792 | 39.074 | 1318.375 | 7447.503 | | 55.1115222 |
| 1793 | 59.704 | 1318.363 | 36163.148 | | 267.607295 |
| 1794 | 30.603 | 1314.452 | 239448.859 | | 1771.92156 |
| 1795 | 21.917 | 1312.679 | 77039.18 | | 570.089932 |
| 1796 | 26.005 | 1312.635 | 141686.484 | | 1048.47998 |
| 1797 | 52.047 | 1311.498 | 829.617 | | 6.1391658 |
| 1798 | 25.103 | 1310.563 | 660235.938 | | 4885.74594 |
| 1799 | 60.227 | 1306.401 | 3903.396 | | 28.8851304 |
| 1800 | 45.07 | 1303.966 | 34127.25 | | 252.54165 |
| 1801 | 25.985 | 1302.62 | 19980.832 | | 147.858157 |
| 1802 | 51.644 | 1302.438 | 2354.193 | | 17.4210282 |
| 1803 | 59.746 | 1302.393 | 45806.871 | | 338.970845 |
| 1804 | 22.252 | 1301.272 | 57392.363 | | 424.703486 |
| 1805 | 54.153 | 1300.353 | 8612.625 | | 63.733425 |
| 1806 | 21.95 | 1299.873 | 83524.633 | | 618.082284 |
| 1807 | 51.573 | 1299.424 | 30573.758 | | 226.245809 |
| 1808 | 53.107 | 1299.416 | 25302.227 | | 187.23648 |
| 1809 | 19.258 | 1298.642 | 21156.547 | | 156.558448 |
| 1810 | 59.374 | 1298.359 | 12482.001 | | 92.3668074 |
| 1811 | 45.03 | 1297.919 | 185944.781 | | 1375.99138 |
| 1812 | 19.022 | 1296.685 | 28328.535 | | 209.631159 |
| 1813 | 59.676 | 1295.779 | 9187.956 | | 67.9908744 |
| 1814 | 28.449 | 1295.485 | 170789.578 | | 1263.84288 |
| 1815 | 25.008 | 1294.604 | 46179.98 | | 341.731852 |
| 1816 | 30.249 | 1293.53 | 103213.75 | | 763.78175 |
| 1817 | 23.194 | 1292.502 | 19937.287 | | 147.535924 |
| 1818 | 30.646 | 1290.433 | 8808.077 | | 65.1797698 |
| 1819 | 28.43 | 1289.456 | 18305.74 | | 135.462476 |
| 1820 | 58.946 | 1289.403 | 1384.637 | | 10.2463138 |
| 1821 | 59.627 | 1289.387 | 2301.153 | | 17.0285322 |
| 1822 | 19.693 | 1289.386 | 4377.44 | | 32.393056 |
| 1823 | 25.244 | 1288.534 | 2999592.25 | | 22196.9827 |
| 1824 | 59.353 | 1286.338 | 15882.02 | | 117.526948 |
| 1825 | 30.82 | 1283.472 | 39585.328 | | 292.931427 |
| 1826 | 51.523 | 1283.445 | 6039.294 | | 44.6907756 |
| 1827 | 53.186 | 1283.43 | 8806.266 | | 65.1663684 |
| 1828 | 34.802 | 1283.335 | 8705.754 | | 64.4225796 |
| 1829 | 59.03 | 1282.394 | 15341.116 | | 113.524258 |
| 1830 | 55.529 | 1282.378 | 7111.861 | | 52.6277714 |
| 1831 | 45.022 | 1280.883 | 30969.486 | | 229.174196 |
| 1832 | 19.518 | 1280.665 | 11716.79 | | 86.704246 |
| 1833 | 59.668 | 1280.411 | 49052 | | 362.9848 |
| 1834 | 28.452 | 1279.513 | 51832.395 | | 383.559723 |
| 1835 | 51.44 | 1277.454 | 82467.625 | | 610.260425 |
| 1836 | 52.927 | 1277.446 | 43342.348 | | 320.733375 |
| 1837 | 45.003 | 1276.982 | 70758.93 | | 523.616082 |
| 1838 | 30.44 | 1276.942 | 232610.75 | | 1721.31955 |
| 1839 | 50.608 | 1276.907 | 1109.392 | | 8.2095008 |
| 1840 | 23.116 | 1276.515 | 122081.953 | | 903.406452 |
| 1841 | 44.995 | 1274.825 | 81134.063 | | 600.392066 |
| 1842 | 26.295 | 1272.61 | 41668.891 | | 308.349793 |
| 1843 | 25.135 | 1272.604 | 1010380.88 | | 7476.81848 |
| 1844 | 20.731 | 1272.48 | 34951.859 | | 258.643757 |
| 1845 | 30.456 | 1272.332 | 289698.906 | | 2143.7719 |
| 1846 | 31.95 | 1270.569 | 58033.617 | | 429.448766 |
| 1847 | 54.813 | 1270.498 | 1479.774 | | 10.9503276 |
| 1848 | 59.96 | 1270.475 | 3445.866 | | 25.4994084 |
| 1849 | 57.989 | 1270.447 | 1281.669 | | 9.4843506 |
| 1850 | 59.188 | 1270.424 | 5638.458 | | 41.7245892 |
| 1851 | 37.091 | 1269.475 | 1353.805 | | 10.018157 |
| 1852 | 40.97 | 1269.437 | 2427.428 | | 17.9629672 |
| 1853 | 35.834 | 1269.418 | 2728.887 | | 20.1937638 |
| 1854 | 40.087 | 1269.413 | 1651.728 | | 12.2227872 |
| 1855 | 39.127 | 1269.409 | 5886.622 | | 43.5610028 |
| 1856 | 36.13 | 1269.399 | 4146.72 | | 30.685728 |
| 1857 | 30.571 | 1267.515 | 21216.277 | | 157.00045 |
| 1858 | 59.349 | 1266.399 | 6219.467 | | 46.0240558 |
| 1859 | 54.894 | 1266.362 | 4477.317 | | 33.1321458 |
| 1860 | 22.184 | 1263.606 | 94234.227 | | 697.33328 |
| 1861 | 59.935 | 1263.437 | 57252.406 | | 423.667804 |
| 1862 | 34.938 | 1263.312 | 16850.229 | | 124.691695 |
| 1863 | 37.489 | 1262.942 | 10585.427 | | 78.3321598 |
| 1864 | 55.342 | 1262.696 | 14468.394 | | 107.066116 |
| 1865 | 52.525 | 1262.35 | 8346.335 | | 61.762879 |
| 1866 | 39.287 | 1262.338 | 12846.079 | | 95.0609846 |
| 1867 | 40.214 | 1262.335 | 8975.618 | | 66.4195732 |
| 1868 | 58.04 | 1262.315 | 3522.354 | | 26.0654196 |
| 1869 | 21.765 | 1262.289 | 123903.367 | | 916.884916 |
| 1870 | 51.525 | 1261.458 | 99836.336 | | 738.788886 |
| 1871 | 53.043 | 1261.455 | 83416.992 | | 617.285741 |
| 1872 | 23.116 | 1260.537 | 46480.016 | | 343.952118 |
| 1873 | 34.799 | 1259.311 | 14362.161 | | 106.279991 |
| 1874 | 28.459 | 1257.534 | 298317.219 | | 2207.54742 |
| 1875 | 31.559 | 1255.699 | 116446.039 | | 861.700689 |
| 1876 | 30.3 | 1255.583 | 846248.25 | | 6262.23705 |
| 1877 | 32.837 | 1255.567 | 35492.637 | | 262.645514 |
| 1878 | 33.291 | 1255.561 | 28837.1 | | 213.39454 |
| 1879 | 51.244 | 1255.489 | 2861.904 | | 21.1780896 |
| 1880 | 32.024 | 1254.973 | 95395.18 | | 705.924332 |
| 1881 | 23.278 | 1254.568 | 121895.391 | | 902.025893 |
| 1882 | 59.74 | 1254.366 | 5949.095 | | 44.023303 |
| 1883 | 57.787 | 1254.364 | 4684.266 | | 34.6635684 |
| 1884 | 58.949 | 1254.347 | 2524.573 | | 18.6818402 |
| 1885 | 57.081 | 1254.319 | 8885.642 | | 65.7537508 |
| 1886 | 55.421 | 1254.301 | 4541.622 | | 33.6080028 |
| 1887 | 45.012 | 1253.896 | 147699.641 | | 1092.97734 |
| 1888 | 28.407 | 1251.496 | 141158.859 | | 1044.57556 |
| 1889 | 25.249 | 1250.631 | 3974873.75 | 34371.53 | 29414.0658 |
| 1890 | 58.643 | 1250.455 | 11072.911 | | 81.9395414 |
| 1891 | 18.98 | 1249.756 | 27491.875 | | 203.439875 |
| 1892 | 31.656 | 1248.538 | 26623.307 | | 197.012472 |
| 1893 | 32.92 | 1247.361 | 41003.309 | | 303.424487 |
| 1894 | 51.498 | 1245.498 | 8761.765 | | 64.837061 |
| 1895 | 53.085 | 1245.476 | 44366.938 | | 328.315341 |
| 1896 | 19.632 | 1244.64 | 30831.113 | | 228.150236 |
| 1897 | 59.714 | 1242.427 | 56404.984 | | 417.396882 |
| 1898 | 56.779 | 1241.078 | 2758.177 | | 20.4105098 |
| 1899 | 30.278 | 1239.607 | 317820.063 | | 2351.86847 |
| 1900 | 31.547 | 1239.563 | 59098.148 | | 437.326295 |
| 1901 | 28.472 | 1239.526 | 69276.641 | | 512.647143 |
| 1902 | 51.321 | 1239.492 | 69059.977 | | 511.04383 |
| 1903 | 52.866 | 1239.474 | 32897.426 | | 243.440952 |
| 1904 | 60.102 | 1238.604 | 6424.091 | | 47.5382734 |
| 1905 | 23.198 | 1238.572 | 492393.188 | | 3643.70959 |
| 1906 | 25.057 | 1238.532 | 9391.734 | | 69.4988316 |
| 1907 | 37.49 | 1237.332 | 6531.968 | | 48.3365632 |
| 1908 | 45.022 | 1236.87 | 46682.961 | | 345.453911 |
| 1909 | 19.717 | 1236.611 | 7412.524 | | 54.8526776 |
| 1910 | 40.236 | 1236.283 | 8209.991 | | 60.7539334 |
| 1911 | 52.172 | 1235.898 | 888.905 | | 6.577897 |
| 1912 | 19.301 | 1235.868 | 4725.906 | | 34.9717044 |
| 1913 | 30.417 | 1234.37 | 856949.25 | | 6341.42445 |
| 1914 | 31.611 | 1233.532 | 122064.055 | | 903.274007 |
| 1915 | 25.133 | 1232.595 | 24227.906 | | 179.286504 |
| 1916 | 21.214 | 1231.482 | 60734.938 | | 449.438541 |
| 1917 | 32.839 | 1231.084 | 60950.719 | | 451.035321 |
| 1918 | 42.855 | 1230.887 | 154045.125 | | 1139.93393 |
| 1919 | 30.565 | 1229.563 | 91573.625 | | 677.644825 |
| 1920 | 22.209 | 1228.627 | 18700.881 | | 138.386519 |
| 1921 | 60.379 | 1227.376 | 3367.176 | | 24.9171024 |
| 1922 | 52.522 | 1227.324 | 6150.93 | | 45.516882 |
| 1923 | 51.79 | 1227.323 | 4718.815 | | 34.919231 |
| 1924 | 49.813 | 1227.305 | 11513.576 | | 85.2004624 |
| 1925 | 40.192 | 1227.301 | 7194.075 | | 53.236155 |
| 1926 | 59.636 | 1226.901 | 24848.395 | | 183.878123 |
| 1927 | 21.859 | 1224.549 | 44181.957 | | 326.946482 |
| 1928 | 30.406 | 1224.415 | 69779.727 | | 516.36998 |
| 1929 | 23.094 | 1222.581 | 91712.016 | | 678.668918 |
| 1930 | 51.595 | 1222.471 | 3662.568 | | 27.1030032 |
| 1931 | 30.29 | 1218.52 | 476128.844 | | 3523.35345 |
| 1932 | 31.528 | 1217.669 | 163110.922 | | 1207.02082 |
| 1933 | 25.119 | 1217.539 | 169168.734 | | 1251.84863 |
| 1934 | 23.259 | 1216.597 | 263113.625 | | 1947.04083 |
| 1935 | 44.996 | 1215.355 | 16854.1 | | 124.72034 |
| 1936 | 31.638 | 1211.447 | 117543.125 | | 869.819125 |
| 1937 | 55.57 | 1210.742 | 2894.646 | | 21.4203804 |
| 1938 | 20.68 | 1210.615 | 33522.359 | | 248.065457 |
| 1939 | 31.023 | 1210.478 | 47086.57 | | 348.440618 |
| 1940 | 60.002 | 1210.378 | 2460.045 | | 18.204333 |
| 1941 | 59.452 | 1210.36 | 2822.82 | | 20.888868 |
| 1942 | 53.367 | 1210.321 | 12792.264 | | 94.6627536 |
| 1943 | 56.782 | 1210.2 | 4122.384 | | 30.5056416 |
| 1944 | 45.004 | 1209.865 | 121766.766 | | 901.074068 |
| 1945 | 25.804 | 1209.47 | 31311.213 | | 231.702976 |
| 1946 | 20.729 | 1206.596 | 10410.285 | | 77.036109 |
| 1947 | 51.214 | 1205.491 | 3944.53 | | 29.189522 |
| 1948 | 31.553 | 1203.616 | 23597.301 | | 174.620027 |
| 1949 | 49.516 | 1203.133 | 8821.522 | | 65.2792628 |
| 1950 | 30.195 | 1202.589 | 39710.629 | | 293.858655 |
| 1951 | 50.689 | 1202.365 | 25647.943 | | 189.794778 |
| 1952 | 51.135 | 1201.518 | 6787.769 | | 50.2294906 |
| 1953 | 42.67 | 1200.783 | 72117.422 | | 533.668923 |
| 1954 | 23.167 | 1200.609 | 1313535.88 | | 9720.16548 |
| 1955 | 25.206 | 1200.547 | 16658.26 | | 123.271124 |
| 1956 | 46.71 | 1200.503 | 19274.873 | | 142.63406 |
| 1957 | 30.044 | 1198.562 | 22444.525 | | 166.089485 |
| 1958 | 59.87 | 1198.361 | 4803.752 | | 35.5477648 |
| 1959 | 59.432 | 1198.353 | 4145.694 | | 30.6781356 |
| 1960 | 19.706 | 1198.222 | 12631.365 | | 93.472101 |
| 1961 | 21.013 | 1197.031 | 87272.023 | | 645.81297 |
| 1962 | 30.406 | 1196.417 | 620780.5 | | 4593.7757 |
| 1963 | 60.6 | 1196.352 | 7650.589 | | 56.6143586 |
| 1964 | 31.578 | 1195.579 | 464075.188 | | 3434.15639 |
| 1965 | 59.222 | 1190.363 | 5847.735 | | 43.273239 |
| 1966 | 53.252 | 1188.334 | 55063.84 | | 407.472416 |
| 1967 | 59.825 | 1187.418 | 3255.71 | | 24.092254 |
| 1968 | 21.882 | 1186.597 | 192521.25 | | 1424.65725 |
| 1969 | 60.255 | 1186.382 | 6004.868 | | 44.4360232 |
| 1970 | 30.174 | 1185.657 | 58927.867 | | 436.066216 |
| 1971 | 42.453 | 1184.524 | 53516.508 | | 396.022159 |
| 1972 | 59.174 | 1184.402 | 4914.623 | | 36.3682102 |
| 1973 | 21.325 | 1183.635 | 234638.141 | | 1736.32224 |
| 1974 | 59.7 | 1183.443 | 7250.531 | | 53.6539294 |
| 1975 | 31.801 | 1182.586 | 11399.009 | | 84.3526666 |
| 1976 | 31.299 | 1181.569 | 94998.031 | | 702.985429 |
| 1977 | 49.745 | 1181.254 | 3827.925 | | 28.326645 |
| 1978 | 56.778 | 1181.221 | 3641.417 | | 26.9464858 |
| 1979 | 60.62 | 1180.368 | 5690.042 | | 42.1063108 |
| 1980 | 50.733 | 1180.335 | 38225.785 | | 282.870809 |
| 1981 | 31.583 | 1179.597 | 236420.203 | | 1749.5095 |
| 1982 | 19.143 | 1178.579 | 11057.592 | | 81.8261808 |
| 1983 | 20.779 | 1178.495 | 131686.141 | | 974.477443 |
| 1984 | 51.247 | 1178.431 | 6476.849 | | 47.9286826 |
| 1985 | 41.654 | 1178.397 | 6623.212 | | 49.0117688 |
| 1986 | 56.782 | 1178.216 | 4255.032 | | 31.4872368 |
| 1987 | 25.333 | 1175.511 | 22551.197 | | 166.878858 |
| 1988 | 24.101 | 1172.463 | 51734.363 | | 382.834286 |
| 1989 | 53.214 | 1172.348 | 38737.391 | | 286.656693 |
| 1990 | 44.949 | 1170.829 | 10637.769 | | 78.7194906 |
| 1991 | 22.67 | 1169.26 | 18541.186 | | 137.204776 |
| 1992 | 54.977 | 1169.167 | 7155.165 | | 52.948221 |
| 1993 | 60.253 | 1166.411 | 2237.358 | | 16.5564492 |
| 1994 | 53.22 | 1166.339 | 140311.391 | | 1038.30429 |
| 1995 | 31.322 | 1165.301 | 58614.336 | | 433.746086 |
| 1996 | 31.542 | 1163.587 | 34373.684 | | 254.365262 |
| 1997 | 42.864 | 1163.561 | 317640.313 | | 2350.53832 |
| 1998 | 20.221 | 1162.602 | 72301.289 | | 535.029539 |
| 1999 | 30.294 | 1160.457 | 18931.172 | | 140.090673 |
| 2000 | 20.533 | 1159.644 | 11926.854 | | 88.2587196 |
| 2001 | 25.361 | 1158.633 | 29713.969 | | 219.883371 |
| 2002 | 31.529 | 1157.615 | 44626.973 | | 330.2396 |
| 2003 | 24.123 | 1156.492 | 33221.211 | | 245.836961 |
| 2004 | 19.431 | 1154.638 | 47105.656 | | 348.581854 |
| 2005 | 31.311 | 1154.523 | 72400.438 | | 535.763241 |
| 2006 | 23.924 | 1152.527 | 201380.953 | | 1490.21905 |
| 2007 | 30.384 | 1152.393 | 58578.801 | | 433.483127 |
| 2008 | 21.985 | 1150.625 | 37468.707 | | 277.268432 |
| 2009 | 53.194 | 1150.361 | 120706.516 | | 893.228218 |
| 2010 | 30.027 | 1149.312 | 119640.453 | | 885.339352 |
| 2011 | 42.541 | 1147.575 | 30441.695 | | 225.268543 |
| 2012 | 23.629 | 1144.649 | 29225.717 | | 216.270306 |
| 2013 | 31.311 | 1143.136 | 23237.896 | | 171.96043 |
| 2014 | 24.001 | 1142.545 | 23198.781 | | 171.670979 |
| 2015 | 50.597 | 1142.373 | 118697.242 | | 878.359591 |
| 2016 | 23.275 | 1141.59 | 47004.074 | | 347.830148 |
| 2017 | 21.014 | 1140.488 | 174479.281 | | 1291.14668 |
| 2018 | 41.649 | 1140.415 | 5001.429 | | 37.0105746 |
| 2019 | 19.442 | 1138.647 | 71437.797 | | 528.639698 |
| 2020 | 31.367 | 1138.553 | 111894.984 | | 828.022882 |
| 2021 | 25.359 | 1137.454 | 68654.234 | | 508.041332 |
| 2022 | 53.625 | 1137.364 | 4344.629 | | 32.1502546 |
| 2023 | 23.802 | 1136.534 | 21588.393 | | 159.754108 |
| 2024 | 25.107 | 1135.542 | 37640.664 | | 278.540914 |
| 2025 | 22.904 | 1134.648 | 71461.75 | | 528.81695 |
| 2026 | 24.113 | 1134.504 | 347155.5 | | 2568.9507 |
| 2027 | 60.091 | 1134.459 | 5499.415 | | 40.695671 |
| 2028 | 59.353 | 1134.437 | 3032.865 | | 22.443201 |
| 2029 | 53.188 | 1134.391 | 56996.746 | | 421.77592 |
| 2030 | 51.575 | 1134.21 | 9499.726 | | 70.2979724 |
| 2031 | 29.971 | 1133.34 | 41626.363 | | 308.035086 |
| 2032 | 20.936 | 1128.605 | 47002.93 | | 347.821682 |
| 2033 | 28.199 | 1128.485 | 105328.227 | | 779.42888 |
| 2034 | 53.209 | 1128.38 | 354067.813 | | 2620.10182 |
| 2035 | 50.589 | 1126.389 | 28851.957 | | 213.504482 |
| 2036 | 31.552 | 1125.231 | 11812.239 | | 87.4105686 |
| 2037 | 41.868 | 1125.221 | 15596.393 | | 115.413308 |
| 2038 | 32.903 | 1125.218 | 126022.156 | | 932.563954 |
| 2039 | 31.908 | 1125.217 | 25750.453 | | 190.553352 |
| 2040 | 39.864 | 1125.21 | 6728.264 | | 49.7891536 |
| 2041 | 36.571 | 1125.206 | 22293.379 | | 164.971005 |
| 2042 | 39.556 | 1125.202 | 16396.361 | | 121.333071 |
| 2043 | 26.016 | 1125.161 | 31188 | | 230.7912 |
| 2044 | 29.167 | 1125.148 | 41243.914 | | 305.204964 |
| 2045 | 38.059 | 1124.829 | 29475.412 | | 218.118049 |
| 2046 | 31.341 | 1122.552 | 34561.762 | | 255.757039 |
| 2047 | 23.72 | 1122.456 | 36783.59 | | 272.198566 |
| 2048 | 44.964 | 1121.808 | 44746.047 | | 331.120748 |
| 2049 | 18.812 | 1119.758 | 5198.635 | | 38.469899 |
| 2050 | 24.134 | 1118.525 | 119093.102 | | 881.288955 |
| 2051 | 25.151 | 1118.499 | 30842.926 | | 228.237652 |
| 2052 | 23.899 | 1114.554 | 208412.047 | | 1542.24915 |
| 2053 | 29.989 | 1111.346 | 183555.891 | | 1358.31359 |
| 2054 | 28.181 | 1110.479 | 70840.586 | | 524.220336 |
| 2055 | 51.147 | 1110.414 | 125425.094 | | 928.145696 |
| 2056 | 20.151 | 1108.578 | 25817.43 | | 191.048982 |
| 2057 | 23.801 | 1106.903 | 187048.984 | | 1384.16248 |
| 2058 | 50.557 | 1104.421 | 171816.922 | | 1271.44522 |
| 2059 | 58.615 | 1102.375 | 6383.561 | | 47.2383514 |
| 2060 | 51.593 | 1101.214 | 1985.06 | | 14.689444 |
| 2061 | 49.777 | 1100.828 | 2256.389 | | 16.6972786 |
| 2062 | 19.219 | 1100.543 | 6005.286 | | 44.4391164 |
| 2063 | 21.831 | 1100.522 | 38794.496 | | 287.07927 |
| 2064 | 25.329 | 1099.552 | 13957.11 | | 103.282614 |
| 2065 | 53.545 | 1099.417 | 30059.629 | | 222.441255 |
| 2066 | 21.401 | 1098.745 | 112194.32 | | 830.237968 |
| 2067 | 52.17 | 1098.418 | 3632.612 | | 26.8813288 |
| 2068 | 20.123 | 1097.551 | 4589.071 | | 33.9591254 |
| 2069 | 24.128 | 1096.552 | 522675.844 | | 3867.80125 |
| 2070 | 53.285 | 1096.434 | 26559.404 | | 196.53959 |
| 2071 | 51.387 | 1096.263 | 109630.906 | | 811.268704 |
| 2072 | 29.987 | 1095.392 | 58529 | | 433.1146 |
| 2073 | 19.018 | 1094.643 | 6701.293 | | 49.5895682 |
| 2074 | 20.501 | 1091.546 | 15707.116 | | 116.232658 |
| 2075 | 23.598 | 1090.488 | 10901.328 | | 80.6698272 |
| 2076 | 53.19 | 1090.433 | 338706.531 | | 2506.42833 |
| 2077 | 20.982 | 1082.62 | 65489.449 | | 484.621923 |
| 2078 | 21.397 | 1082.584 | 32035.098 | | 237.059725 |
| 2079 | 19.289 | 1080.554 | 12653.619 | | 93.6367806 |
| 2080 | 53.408 | 1076.445 | 4457.142 | | 32.9828508 |
| 2081 | 53.657 | 1076.436 | 3256.97 | | 24.101578 |
| 2082 | 52.905 | 1076.431 | 2378.771 | | 17.6029054 |
| 2083 | 20.569 | 1075.555 | 86250.758 | | 638.255609 |
| 2084 | 29.991 | 1073.413 | 40851.953 | | 302.304452 |
| 2085 | 23.494 | 1068.813 | 147886.688 | | 1094.36149 |
| 2086 | 21.443 | 1068.575 | 62297.086 | | 460.998436 |
| 2087 | 18.988 | 1068.472 | 18219.826 | | 134.826712 |
| 2088 | 23.8 | 1064.568 | 7053.475 | | 52.195715 |
| 2089 | 20.279 | 1063.503 | 21330.266 | | 157.843968 |
| 2090 | 19.116 | 1062.663 | 4397.519 | | 32.5416406 |
| 2091 | 32.024 | 1060.467 | 45340.27 | | 335.517998 |
| 2092 | 60.603 | 1060.416 | 6794.908 | | 50.2823192 |
| 2093 | 52.255 | 1058.393 | 12166.359 | | 90.0310566 |
| 2094 | 54.069 | 1058.366 | 9593.294 | | 70.9903756 |
| 2095 | 49.741 | 1057.321 | 5347.421 | | 39.5709154 |
| 2096 | 23.962 | 1054.469 | 82210.703 | | 608.359202 |
| 2097 | 23.588 | 1051.388 | 89102.742 | | 659.360291 |
| 2098 | 30.345 | 1044.353 | 4195.99 | | 31.050326 |
| 2099 | 23.401 | 1040.537 | 10620.867 | | 78.5944158 |
| 2100 | 20.577 | 1032.597 | 21339.391 | | 157.911493 |
| 2101 | 24.092 | 1025.286 | 62953.594 | | 465.856596 |
| 2102 | 19.87 | 1018.527 | 7472.115 | | 55.293651 |
| 2103 | 23.953 | 1016.515 | 121646.805 | | 900.186357 |
| 2104 | 30.418 | 1016.354 | 134689.156 | | 996.699754 |
| 2105 | 20.528 | 1015.594 | 21665.502 | | 160.324715 |
| 2106 | 23.573 | 1013.438 | 115542.805 | | 855.016757 |
| 2107 | 30.392 | 1011.276 | 33455.215 | | 247.568591 |
| 2108 | 18.922 | 1010.399 | 9148.562 | | 67.6993588 |
| 2109 | 20.423 | 1006.679 | 8503.45 | | 62.92553 |
| 2110 | 47.1 | 998.211 | 46778.898 | | 346.163845 |
| 2111 | 23.462 | 995.402 | 32905.055 | | 243.497407 |
| 2112 | 51.388 | 991.214 | 2437.004 | | 18.0338296 |
| 2113 | 21.942 | 988.585 | 32196.633 | | 238.255084 |
| 2114 | 20.878 | 988.565 | 12280.876 | | 90.8784824 |
| 2115 | 29.367 | 987.262 | 48744.594 | | 360.709996 |
| 2116 | 23.484 | 984.509 | 27134.879 | | 200.798105 |
| 2117 | 47.966 | 984.342 | 3780.828 | | 27.9781272 |
| 2118 | 23.398 | 981.665 | 20057.672 | | 148.426773 |
| 2119 | 30.895 | 981.502 | 25569.025 | | 189.210785 |
| 2120 | 19.773 | 978.532 | 4305.65 | | 31.86181 |
| 2121 | 30.386 | 973.323 | 32013.334 | | 236.898672 |
| 2122 | 29.291 | 971.253 | 5491.579 | | 40.6376846 |
| 2123 | 20.901 | 969.104 | 41305.473 | | 305.6605 |
| 2124 | 25.141 | 965.491 | 48757.68 | | 360.806832 |
| 2125 | 24.072 | 964.44 | 7556.274 | | 55.9164276 |
| 2126 | 20.829 | 963.206 | 16317.524 | | 120.749678 |
| 2127 | 28.81 | 962.49 | 19231.721 | | 142.314735 |
| 2128 | 20.219 | 960.462 | 9783.454 | | 72.3975596 |
| 2129 | 23.379 | 956.597 | 24888.623 | | 184.17581 |
| 2130 | 22.899 | 956.484 | 12570.926 | | 93.0248524 |
| 2131 | 59.245 | 955.564 | 1391.343 | | 10.2959382 |
| 2132 | 19.095 | 954.938 | 8325.797 | | 61.6108978 |
| 2133 | 20.123 | 952.494 | 5814.21 | | 43.025154 |
| 2134 | 60.591 | 950.571 | 4099.453 | | 30.3359522 |
| 2135 | 40.291 | 950.357 | 19953.055 | | 147.652607 |
| 2136 | 41.194 | 950.357 | 47130.363 | | 348.764686 |
| 2137 | 24.004 | 949.406 | 63025.402 | | 466.387975 |
| 2138 | 29.386 | 949.317 | 56691.895 | | 419.520023 |
| 2139 | 51.536 | 948.37 | 24475.225 | | 181.116665 |
| 2140 | 60.327 | 947.639 | 4107.147 | | 30.3928878 |
| 2141 | 23.951 | 945.481 | 43559.406 | | 322.339604 |
| 2142 | 20.894 | 944.588 | 148397.719 | | 1098.14312 |
| 2143 | 30.047 | 944.458 | 13261.684 | | 98.1364616 |
| 2144 | 23.976 | 933.488 | 10559.713 | | 78.1418762 |
| 2145 | 20.085 | 931.546 | 45600.914 | | 337.446764 |
| 2146 | 18.961 | 926.553 | 4596.708 | | 34.0156392 |
| 2147 | 20.898 | 925.264 | 49014.879 | | 362.710105 |
| 2148 | 20.978 | 920.557 | 5005.199 | | 37.0384726 |
| 2149 | 18.938 | 918.591 | 4865.919 | | 36.0078006 |
| 2150 | 56.883 | 918.079 | 1112.862 | | 8.2351788 |
| 2151 | 51.665 | 915.677 | 1583.223 | | 11.7158502 |
| 2152 | 52.923 | 915.395 | 2432.005 | | 17.996837 |
| 2153 | 29.904 | 914.917 | 25552.555 | | 189.088907 |
| 2154 | 40.82 | 912.413 | 454771.625 | | 3365.31003 |
| 2155 | 23.975 | 911.5 | 11979.838 | | 88.6508012 |
| 2156 | 18.986 | 911.287 | 4659.146 | | 34.4776804 |
| 2157 | 29.339 | 906.271 | 22190.25 | | 164.20785 |
| 2158 | 51.09 | 902.979 | 700.182 | | 5.1813468 |
| 2159 | 20.832 | 902.486 | 52433.75 | | 388.00975 |
| 2160 | 49.645 | 900.36 | 24044.1 | | 177.92634 |
| 2161 | 24.096 | 898.383 | 45429.156 | | 336.175754 |
| 2162 | 56.504 | 897.659 | 5498.265 | | 40.687161 |
| 2163 | 56.278 | 896.829 | 1467.63 | | 10.860462 |
| 2164 | 22.577 | 896.41 | 21323.018 | | 157.790333 |
| 2165 | 19.262 | 895.584 | 4205.946 | | 31.1240004 |
| 2166 | 23.992 | 882.405 | 17175.494 | | 127.098656 |
| 2167 | 55.786 | 880.774 | 2373.386 | | 17.5630564 |
| 2168 | 59.474 | 879.635 | 1745.675 | | 12.917995 |
| 2169 | 19.949 | 879.567 | 5377.363 | | 39.7924862 |
| 2170 | 29.754 | 876.473 | 23381.605 | | 173.023877 |
| 2171 | 42.753 | 875.379 | 114573.516 | | 847.844018 |
| 2172 | 21.028 | 872.459 | 5747.54 | | 42.531796 |
| 2173 | 19.956 | 868.588 | 5911.415 | | 43.744471 |
| 2174 | 51.593 | 867.774 | 1445.617 | | 10.6975658 |
| 2175 | 55.321 | 867.675 | 724.974 | | 5.3648076 |
| 2176 | 59.198 | 867.635 | 1190.664 | | 8.8109136 |
| 2177 | 58.799 | 867.622 | 2502.561 | | 18.5189514 |
| 2178 | 20.829 | 852.468 | 7367.667 | | 54.5207358 |
| 2179 | 49.047 | 846.946 | 1713.276 | | 12.6782424 |
| 2180 | 57.438 | 846.946 | 2112.215 | | 15.630391 |
| 2181 | 60.566 | 846.924 | 2585.42 | | 19.132108 |
| 2182 | 22.644 | 846.426 | 1215.356 | | 8.9936344 |
| 2183 | 29.086 | 841.303 | 8721.339 | | 64.5379086 |
| 2184 | 53.187 | 837.301 | 11143.638 | | 82.4629212 |
| 2185 | 55.22 | 835.737 | 611.264 | | 4.5233536 |
| 2186 | 54.381 | 832.253 | 1154.893 | | 8.5462082 |
| 2187 | 59.347 | 824.672 | 836.045 | | 6.186733 |
| 2188 | 20.776 | 824.421 | 2193.866 | | 16.2346084 |
| 2189 | 59.758 | 824.025 | 5333.992 | | 39.4715408 |
| 2190 | 58.792 | 823.646 | 7006.314 | | 51.8467236 |
| 2191 | 53.249 | 821.319 | 3106.751 | | 22.9899574 |
| 2192 | 60.055 | 815.685 | 1288.741 | | 9.5366834 |
| 2193 | 29.084 | 815.416 | 2310.018 | | 17.0941332 |
| 2194 | 50.654 | 813.317 | 13369.253 | | 98.9324722 |
| 2195 | 53.192 | 799.334 | 37386.438 | | 276.659641 |

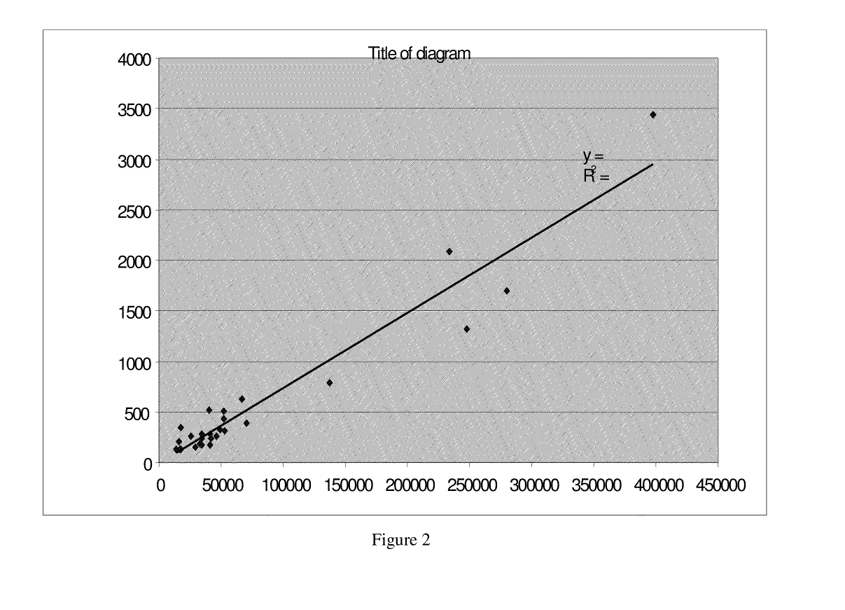

The quality of the regression is shown in FIG. 2.

1. A process for normalizing the concentration of at least one analyte in a urine sample, comprising the following steps:
a) associating at least a portion of the urine sample with an analytical instrument to determine the concentration of at least one analyte in the sample;
b) associating at least a portion of the urine sample with an analytical instrument to determine the concentration of at least one collagen or collagen fragment within the sample, wherein the determining of the concentration of the at least one collagen or collagen fragment comprises 1) separating at least one collagen or collagen fragments from the sample or a portion thereof; 2) performing mass spectrometry; or using a combination of 1) and 2); and
c) normalizing the concentration of said at least one analyte relative to the concentration of said at least one collagen or collagen fragment.

2. The process according to claim 1, wherein the urine sample is subdivided by separation into several subsamples before the concentration of the analyte is determined.

3. The process according to claim 2, wherein said separation is effected by:
chromatographic methods,
adsorption to specific materials, or
electrophoretic methods.

4. The process according to claim 1, wherein said determination of the concentration of analytes and/or collagen or collagen fragments is effected by a UV/Vis detector, fluorescence detector, refractive index detector, diode array detector, mass spectrometer, or gel scanner.

5. The process according to claim 1, wherein said at least one collagen or collagen fragment is identified by its migration or retention time and optionally further suitable parameters including mass spectra, UV/Vis spectra, fluorescence spectra, or affinity for particular ligands including antibodies.

6. The process according to claim 1, wherein said at least one collagen fragment is selected from the peptides of SEQ ID Nos. 1 to 42.

7. The process according to claim 1, wherein the concentrations of at least two collagens or collagen fragments are determined and employed for normalization.

8. The process according to claim 1, wherein said normalization is effected by using a linear regression established with collagen protein standards.

9. The process according to claim 3, wherein the said chromatographic methods is affinity chromatography, ion-exchange chromatography, reversed-phase chromatography, hydrophobic interaction chromatography, or gel filtration.

10. The process according to claim 3, wherein said chromatographic methods use FPLC or HPLC.

11. The process according to claim 3, wherein said adsorption to specific material is effected by a SELDI process or a ClinProt process.

12. The process according to claim 2, wherein said separation is effected by gel electrophoresis or capillary electrophoresis.

13. The process according to claim 1, wherein the analytes and/or collagen or collagen fragments were treated with staining or derivatization prior to the determination steps.

14. The process according to claim 5, wherein the collagen or collagen fragment is further identified by parameters obtained by mass spectra, UV/Vis spectra, fluorescence spectra, or affinity for particular ligands.

15. The process according to claim 1, wherein the concentrations of at least 5 collagens or collagen fragments are determined and employed for normalization.

16. The process according to claim 1, wherein the concentrations of at least 30 collagens or collagen fragments are determined and employed for normalization.

17. The process according to claim 1, wherein the concentrations of at least 50 collagens or collagen fragments are determined and employed for normalization.
